(19) United States
(12) Patent Application Publication
(10) Pub. No.: US 2002/0193327 A1
(43) Pub. Date: Dec. 19, 2002

(54) Vectors for occular transduction and use therefor for genetic therapy

(75) Inventors: Glen R. Nemerow, Encinitas, CA (US); Daniel J. Von Seggern, San Diego, CA (US); Martin Friedlander, Del Mar, CA (US)

Correspondence Address: HELLER EHRMAN WHITE & McAULIFFE LLP, 6th Floor, 4350 La Jolla Village, San Diego, CA, 92122, US

(73) Assignee: The Scripps Research Institute

(21) Appl. No.: 09/847,101

(22) Filed: May 1, 2001

Related U.S. Application Data
(63) Continuation-in-part of application No. 09/562,934, filed on May 1, 2000.

(51) Int. Cl. A61K 48/00; C07H 21/04; C12N 7/00

(52) U.S. Cl. 514/44; 424/93.21; 435/235.1; 536/23.2

(57) ABSTRACT

Adenovirus vector-based gene therapy methods for treating ocular disorders are provided. Adenovirus vectors for therapy of ocular diseases and methods of treatment using the vectors are provided. Compositions, kits, and methods of preparation and use of the vectors for gene therapy are provided.

### RELATED APPLICATIONS

This application is a continuation-in-part of U.S. application Ser. No. 09/562,934, filed May 1, 2000, to Glen R. Nemerow, Daniel Von Seggern,; Martin Friedlander, entitled “VECTORS FOR OCULAR TRANSDUCTION AND USE THEREFOR FOR GENETIC THERAPY”.

This application is related to copending U.S. application Ser. No. 09/482,682 (also filed as International PCT application No. PCT/US00/00265, filed Jan. 14, 2000)), to Daniel Von Seggern, Glen R. Nemerow, Paul Hallenbeck, Susan Stevenson, Yelena Skripchenko, filed Jan. 14, 2000, entitled “Adenovirus Vectors, Packaging Cell Lines,Compositions, and Methods for Preparation and Use,” which is a continuation-in-part of U.S. application Ser. No. 09/423,783 filed Nov. 12, 1999 and claims the benefit of the filing date of U.S. Provisional Application No. 60/115,920 filed Jan. 14, 1999. Where permitted, the contents and subject matter of each application and of the provisional application are incorporated in their entirety herein by reference.

Work described herein was supported by NIH grants EY11431 and HL54352. The government has certain rights in such subject matter.

### FIELD OF INVENTION

The present invention relates to gene therapy, especially to adenovirus vector-based gene therapy. In particular, adenovirus vectors for therapy of ocular diseases and methods of treatment using the vectors are provided. Compositions, kits, and methods of preparation and use of the vectors for gene therapy are provided.

### BACKGROUND OF THE INVENTION

Retinal Dystrophies

The eye is susceptible to a number of hereditary and/or age related degenerative disorders. In the United States, common causes of irreversible blindness or severe loss of vision are retinal dystrophies (see, e.g., Cotlier et al. (1995) Surv. Ophthalmology 40:51-61; Bird (1995) Am. J. Ophthal. 119: 543-562; and Adler (1996) Arch Ophthal 114:79-83). The retina is the sensory tunic of the eye, containing light sensitive receptors, a complex of neurons, and pigmented epithelium, arranged in discrete layers. In humans, the macula is the portion of the retina that lies directly behind the lens. Cones, the photoreceptor cells responsible for central vision, are heavily concentrated in the macula. Central dystrophies, which affect the macula, include Best's disease, age-related macular degeneration, and Stargardt's macular dystrophy. The peripheral retina is composed mainly of rods, which are responsible for side and night vision. Peripheral degenerative retinal diseases include retinitis pigmentosa, choroidemia and Bietti's crystalline dystrophy.

Macular degenerations are a heterogenous group of diseases, characterized by progressive central vision loss and degeneration of the macula and underlying retinal pigmented epithelium. Age-related macular degeneration (ARMD) is the most common form of the disease, affecting an estimated 20% of persons over 75 years of age. ARMD is poorly understood in terms of etiology and pathogenesis. The very late onset of the disease has made genetic mapping particularly difficult. Certain macular degenerative conditions with a clear genetic basis, such as Stargardt's and Best's diseases, share many features with ARMD, but have been more amenable to molecular and genetic analysis.

Hereditary peripheral retinopathies are also relatively common. Retinitis pigmentosa (RP), for example, affects approximately 1.5 million people worldwide. Substantial genetic heterogeneity has been observed in this condition, with over 20 chromosomal loci identified. A predisposition to retinitis pigmentosa can be inherited by autosomal dominant, autosomal recessive, X-linked or digenic mode. Mutations have been identified in seven genes, four of which encode proteins in the rod phototransduction cascade: rhodopsin, alpha and beta subunits of rod cGMP phosphodiesterase, and rod cGMP cation-gated channel protein .alpha. subunit. Mutations in the peripherin/RDS gene have been linked to retinitis pigmentosa and macular degeneration. A single peripherin/RDS mutation apparently caused retinitis pigmentosa, pattern dystrophy and fundus flavimaculatus, in different family members.

In spite of causal heterogeneity, there is significant clinical similarity among RP subtypes. Common signs and symptoms include early electroretinographic abnormalities, ophthalmoscopic findings, and protracted, contiguous expansion of the ring-like scotoma toward the macula, leading to progressively worsening tunnel vision. A recent hypothesis is that active photoreceptor cell death, which is characteristic of these genetically distinct disorders, is mediated by a common induction of apoptosis. It may be possible to treat these conditions by the administration of agents that block induction of apoptosis in photoreceptors, such as neurotrophic factors.

Adenovirus Delivery Vectors

Adenovirus, which is a DNA virus with a 36 kilobase (kb) genome, is very well-characterized and its genetics and genetic organization are understood. The genetic organization of adenoviruses permits substitution of large fragments of viral DNA with foreign DNA. In addition, recombinant adenoviruses are structurally stable and no rearranged viruses are observed after extensive amplification.

Adenoviruses have been employed as delivery vehicles for introducing desired genes into eukaryotic cells. The adenovirus delivers such genes to eukaryotic cells by binding to cellular receptors followed by internalization. The adenovirus fiber protein is responsible for binding to cells. The fiber protein has two domains, a rod-like shaft portion and a globular head portion that contains the receptor binding region. The fiber spike is a homotrimer, and there are 12 spikes per virion. Human adenoviruses bind to and infect a broad range of cultured cell lines and primary tissues from different species.

The 35,000+ base pair (bp) genome of adenovirus type 2 has been sequenced and the predicted amino acid sequences of the major coat proteins (hexon, fiber and penton base) have been described (see, e.g., Neumann et al., Gene 69: 153-157 (1988); Herisse et al., Nuc. Acids Res. 9: 4023-4041 (1981); Roberts et al., J. Biol. Chem. 259: 13968-13975 (1984); Kinloch et al., J. Biol. Chem. 259: 6431-6436 (1984); and Chroboczek et al., Virol. 161: 549-554, 1987).

The 35,935 bp sequence of Ad5 DNA is also known and portions of many other adenovirus genomes have been sequenced. The upper packaging limit for adenovirus virions is about 105% of the wild-type genome length (see, e.g., Bett, et al., J. Virol. 67(10): 5911-21, 1993). Thus, for Ad2 and Ad5, this would be an upper packaging limit of about 38kb of DNA.

Adenovirus DNA also includes inverted terminal repeat sequences (ITRs) ranging in size from about 100 to 150 bp, depending on the serotype. The inverted repeats permit single strands of viral DNA to circularize by base-pairing of their terminal sequences to form base-paired “panhandle” structures that are required for replication of the viral DNA. For efficient packaging, the ITRs and the packaging signal (a few hundred bp in length) comprise the “minimum requirement” for replication and packaging of a genomic nucleic acid into an adenovirus particle. Helper-dependent vectors lacking all viral ORFs but including these essential cis elements (the ITRs and contiguous packaging sequence) have been constructed.

Ad vectors have several distinct advantages as gene delivery vehicles. For example, recombination of such vectors is rare; there are no known associations of human malignancies with adenoviral infections despite common human infection with adenoviruses; the genome may be manipulated to accommodate foreign genes of a fairly substantial size; and host proliferation is not required for expression of adenoviral proteins. Adenovirus (Ad)-based gene delivery vectors efficiently infect many different cells and tissues. This broad tropism, however, means that gene delivery cannot be directed to a specific target cell. A large fraction of intravenously administered adenovirus is retained by the liver, which could lead to undesirable side-effects. Adenovirus may potentiate immune responses. For example, Adenovirus type 5 (Ad5) also transduces dendritic cells, which present antigens very efficiently, thereby possibly exacerbating the immune response against the vector. It has been proposed that vectors with different targeting efficiencies might eliminate these problems, permitting a lower total particle dose and more specific targeting (see, e.g., U.S. application Ser. No. 09/482,682).

The wealth of information on adenovirus structure and mechanism of infection, its efficient infection of nondividing cells, and its large genetic capacity make adenovirus a popular gene therapy vector. The wide expression of receptors to which adenovirus binds makes targeting adenovirus vectors difficult.

Hence there is a need to improve delivery and targeting of adenoviral vectors and also to provide treatments for ocular disorders. Therefore, it is an object herein to provide adenoviral vectors that specifically or selectively target cells in the eye. It is also an object herein to provide these vectors for treatment of ocular disorders.

### SUMMARY OF THE INVENTION

Degenerative ocular diseases, such as, but not limited to, retinitis pigmentosa, Stargardt's disease, diabetic retinopathies, retinal vascularization, and others (see, e.g., Table below), have a genetic basis. Genes expressed in the photoreceptor cells at the back of the retina are implicated in these diseases. Provided herein are recombinant viral vectors for targeting therapeutic products to these cells.

Recombinant adenoviral vectors that include nucleic acid that permits specific binding to these photoreceptors are provided. In particular, the vector particles contain a fiber protein of Ad37 or a modified form thereof. As shown herein, fiber protein from Ad37 permits efficient infection of photoreceptor cells. Fiber proteins from other adenovirus D serotypes may also be used. In addition, the portions of the fiber protein, particularly those that interact with other viral structural proteins, such as penton, may be modified to resemble the viral source of the other structural proteins. As exemplified herein, the recombinant virus provided herein include Ad5 structural components. The N-terminus of the Ad37 fiber protein, which interacts with the penton protein, is modified to resemble the Ad5 fiber protein N-terminus to ensure production of viral particles.

The recombinant adenoviral vectors are intended for gene therapy of diseases in which genes expressed in the photoreceptors are implicated. Such diseases include, but are not limited to, degenerative ocular diseases, such as retinitis pigmentosa and Stargardt's disease. These vectors are also useful for targeting to other ocular cells, such as conjunctival cells, which also bear receptors to which fiber from Ad37 and related serotypes bind.

The vectors will deliver therapeutic agents to the targeted cells for treatment of a variety of disorders (see e.g., Tables 3 and 4, below)). The therapeutic agents are intended for expression in the photoreceptors and for secretion from the photorecptor cells, which are surrounded on one side by choroidal vasculature, and on the other side by retinal vasulature, thereby providing a means for delivery of products. In addition, expression of growth factors, such as VEGF and others, can be used to enhance blood flow to the retina and prevent or slow the degeneration.

Therapeutic agents encoded by the recombinant adenoviral vectors include, but are not limited to, nucleic acid nucleic acid molecules encoding genes that are defective in certain hereditary disorders, nucleic acid molecules that encode antiangiogenics and antitumor agents for treatment of retinal disorders, such as retinoblastomas; nucleic acid molecules encoding trophic factors, such as glial cell line-derived neuroptrophic factor (GDNF) and ciliary neurotrophic factor (CNTF), growth factors and growth factor inhibitors, antiapoptotic factors, such as Bcl-2 (CNTF), antitumor agents, anti-angiogenics, and genes or portions thereof for gene replacement or repair of defective genes. Hence, methods for treatment of inherited and acquired retinal diseases, including diseases involving neovascular and vascular degeneration are provided.

Methods for treating diseases involving genes expressed in photoreceptor cells are provided herein. The methods provided herein are practiced by administration of the recombinant viral vectors by any means suitable for delivery to the photoreceptors. A preferred mode of administration is intraocular injection including intravitreal and subretinal injection. Other modes of administration include, but are not limited to, intrascleral, periorbital and intravenous administration. The vectors also can include photoreceptor-specific promoters thereby providing a means, not only for specific targeting of expression in these cells, but also for photoreceptor-restricted transgene expression.

### DETAILED DESCRIPTION OF THE INVENTION

A. Definitions

Unless defined otherwise, all technical and scientific terms used herein have the same meaning as is commonly understood by one of skill in the art to which this invention belongs. All patents, applications, published applications and other publications and sequences from GenBank and other data bases referred to anywhere in the disclosure herein are incorporated by reference in their entirety.

As used herein, the amino acids, which occur in the various amino acid sequences appearing herein, are identified according to their three-letter or one-letter abbreviations. The nucleotides, which occur in the various DNA fragments, are designated with the standard single-letter designations used routinely in the art (see, Table 1).

As used herein, amino acid residue refers to an amino acid formed upon chemical digestion (hydrolysis) of a polypeptide at its peptide linkages. The amino acid residues described herein are preferably in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. § § 1.821-1.822, abbreviations for amino acid residues are shown in the following Table:

TABLE 1
Table of Correspondence

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino-terminus to carboxyl-terminus. In addition, the phrase “amino acid residue” is broadly defined to include the amino acids listed in the Table of Correspondence and modified and unusual amino acids, such as those referred to in 37 C.F.R. § § 1.821-1.822, and incorporated herein by reference. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

Such substitutions are preferably made in accordance with those set forth in TABLE 2 as follows:

TABLE 2

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, a complementing plasmid describes plasmid vectors that deliver nucleic acids into a packaging cell line for stable integration into a chromosome in the cellular genome.

As used herein, a delivery plasmid is a plasmid vector that carries or delivers nucleic acids encoding a therapeutic gene or gene that encodes a therapeutic product or a precursor thereof or a regulatory gene or other factor that results in a therapeutic effect when delivered in vivo in or into a cell line, such as, but not limited to a packaging cell line, to propagate therapeutic viral vectors.

As used herein, a variety of vectors with different requirements are described. For example, one vector is used to deliver particular nucleic acid molecules into a packaging cell line for stable integration into a chromosome. These types of vectors are generally identified herein as complementing plasmids. A further type of vector described herein carries or delivers nucleic acid molecules in or into a cell line (e.g., a packaging cell line) for the purpose of propagating therapeutic viral vectors; hence, these vectors are generally referred to herein as delivery plasmids. A third “type” of vector described herein is used to carry nucleic acid molecules encoding therapeutic proteins or polypeptides or regulatory proteins or are regulatory sequences to specific cells or cell types in a subject in need of treatment; these vectors are generally identified herein as therapeutic viral vectors or recombinant adenoviral vectors or viral Ad-derived vectors and are in the form of a virus particle encapsulating a viral nucleic acid containing an expression cassette for expressing the therapeutic gene.

As used herein, a DNA or nucleic acid homolog refers to a nucleic acid that includes a preselected conserved nucleotide sequence, such as a sequence encoding a therapeutic polypeptide. By the term “substantially homologous” is meant having at least 80%, preferably at least 90%, most preferably at least 95% homology therewith or a lesser percentage of homology or identity and conserved biological activity or function.

The terms “homology” and “identity” are often used interchangeably. In this regard, percent homology or identity may be determined, for example, by comparing sequence information using a GAP computer program. The GAP program utilizes the alignment method of Needleman and Wunsch (J. Mol. Biol. 48:443 (1970), as revised by Smith and Waterman (Adv. Appl. Math. 2:482 (1981). Briefly, the GAP program defines similarity as the number of aligned symbols (i.e., nucleotides or amino acids) which are similar, divided by the total number of symbols in the shorter of the two sequences. The preferred default parameters for the GAP program may include: (1) a unary comparison matrix (containing a value of 1 for identities and 0 for non-identities) and the weighted comparison matrix of Gribskov and Burgess, Nucl. Acids Res. 14:6745 (1986), as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Whether any two nucleic acid molecules have nucleotide sequences that are at least 80%, 85%, 90%, 95%, 96%, 97%, 98% or 99% “identical” can be determined using known computer algorithms such as the “FAST A” program, using for example, the default parameters as in Pearson and Lipman, Proc. Natl. Acad. Sci. USA 85:2444 (1988). Alternatively the BLAST function of the National Center for Biotechnology Information database may be used to determine identity.

In general, sequences are aligned so that the highest order match is obtained. “Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotides or polypeptide sequences, the term “identity” is well known to skilled artisans (Carillo, H. & Lipton, D., SIAM J Applied Math 48:1073 (1988)). Methods commonly employed to determine identity or similarity between two sequences include, but are not limited to, those disclosed in Guide to Huge Computers, Martin J. Bishop, ed., Academic Press, San Diego, 1994, and Carillo, H. & Lipton, D., SIAM J Applied Math 48:1073 (1988). Methods to determine identity and similarity are codified in computer programs. Preferred computer program methods to determine identity and similarity between two sequences include, but are not limited to, GCG program package (Devereux, J., et al., Nucleic Acids Research 12(I):387 (1984)), BLASTP, BLASTN, FASTA (Atschul, S. F., et al., J Molec Biol 215:403 (1990)).

Therefore, as used herein, the term “identity” represents a comparison between a test and a reference polypeptide or polynucleotide. For example, a test polypeptide may be defined as any polypeptide that is 90% or more identical to a reference polypeptide. As used herein, the term at least “90% identical to” refers to percent identities from 90 to 99.99 relative to the reference polypeptides. Identity at a level of 90% or more is indicative of the fact that, assuming for exemplification purposes a test and reference polynucleotide length of 100 amino acids are compared. No more than 10% (i.e., 10 out of 100) amino acids in the test polypeptide differs from that of the reference polypeptides. Similar comparisons may be made between a test and reference polynucleotides. Such differences may be represented as point mutations randomly distributed over the entire length of an amino acid sequence or they may be clustered in one or more locations of varying length up to the maximum allowable, e.g. 10/100 amino acid difference (approximately 90% identity). Differences are defined as nucleic acid or amino acid substitutions, or deletions.

As used herein, genetic therapy involves the transfer of heterologous DNA to the certain cells, target cells, of a mammal, particularly a human, with a disorder or conditions for which such therapy is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, heterologous DNA is DNA that encodes RNA and proteins that are not normally produced in vivo by the cell in which it is expressed or that mediates or encodes mediators that alter expression of endogenous DNA by affecting transcription, translation, or other regulatable biochemical processes. Heterologous DNA may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

Hence, herein heterologous DNA or foreign DNA, refers to a DNA molecule not present in the exact orientation and position as the counterpart DNA molecule found in the corresponding wild-type adenovirus. It may also refer to a DNA molecule from another organism or species (i.e., exogenous) or from another Ad serotype.

As used herein, a therapeutically effective product is a product that is encoded by heterologous DNA that, upon introduction of the DNA into a host, a product is expressed that effectively ameliorates or eliminates the symptoms, manifestations of an inherited or acquired disease or that cures said disease.

Typically, DNA encoding the desired heterologous DNA is cloned into a plasmid vector and introduced by routine methods, such as calcium-phosphate mediated DNA uptake (see, (1981) Somat. Cell. Mol. Genet. 7:603-61 6) or microinjection, into producer cells, such as packaging cells. After amplification in producer cells, the vectors that contain the heterologous DNA are introduced into selected target cells.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors.” Also included are vectors that allow cloning of cDNA (complementary DNA) from mRNAs produced using reverse transcriptase.

As used herein, a gene is a nucleic acid molecule whose nucleotide sequence encodes RNA or polypeptide. A gene can be either RNA or DNA. Genes may include regions preceding and following the coding region (leader and trailer) as well as intervening sequences (introns) between individual coding segments (exons).

As used herein, tropism with reference to an adenovirus refers to the selective infectivity or binding that is conferred on the particle by the fiber protein, such as by the C-terminus portion that comprises the knob.

As used herein, isolated with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated,” but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith and Johnson, Gene 67:31-40 (1988). The terms isolated and purified are sometimes used interchangeably.

Thus, by “isolated” is meant that the nucleic acid is free of the coding sequences of those genes that, in the naturally-occurring genome of the organism (if any) immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange chromatography, affinity chromatography, density gradient centrifugation and electrophoresis.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

A cell extract that contains the DNA or protein of interest should be understood to mean a homogenate preparation or cell-free preparation obtained from cells that express the protein or contain the DNA of interest. The term “cell extract” is intended to include culture media, especially spent culture media from which the cells have been removed.

As used herein, a packaging cell line is a cell line that provides a missing gene product or its equivalent.

As used herein, an adenovirus viral particle is the minimal structural or functional unit of a virus. A virus can refer to a single particle, a stock of particles or a viral genome. The adenovirus (Ad) particle is relatively complex and may be resolved into various substructures.

As used herein, “penton” or “penton complex” are preferentially used herein to designate a complex of penton base and fiber. The term “penton” may also be used to indicate penton base, as well as penton complex. The meaning of the term “penton” alone should be clear from the context within which it is used.

As used herein, a plasmid refers to an autonomous self-replicating extrachromosomal circular nucleic acid molecule, typically DNA.

As used herein, a post-transcription regulatory element (PRE) is a regulatory element found in viral or cellular messenger RNA that is not spliced, i.e. intronless messages. Examples include, but are not limited to, human hepatitis virus, woodchuck hepatitis virus, the TK gene and mouse histone gene. The PRE may be placed before a polyA sequence and after a heterologous DNA sequence.

As used herein, pseudotyping describes the production of adenoviral vectors having modified capsid protein or capsid proteins from a different serotype than the serotype of the vector itself. One example, is the production of an adenovirus 5 vector particle containing an Ad37 fiber protein. This may be accomplished by producing the adenoviral vector in packaging cell lines expressing different fiber proteins.

As used herein, promoters of interest herein may be inducible or constitutive. Inducible promoters will initiate transcription only in the presence of an additional molecule; constitutive promoters do not require the presence of any additional molecule to regulate gene expression. a regulatable or inducible promoter may also be described as a promoter where the rate or extent of RNA polymerase binding and initiation is modulated by external stimuli. Such stimuli include, but are not limited to various compounds or compositions, light, heat, stress and chemical energy sources. Inducible, suppressible and repressible promoters are considered regulatable promoters. Preferred promoters herein, are promoters that are selectively expressed in ocular cells, particularly photoreceptor cells.

As used herein, receptor refers to a biologically active molecule that specifically or selectively binds to (or with) other molecules. The term “receptor protein” may be used to more specifically indicate the proteinaceous nature of a specific receptor.

As used herein, recombinant refers to any progeny formed as the result of genetic engineering. This may also be used to describe a virus formed by recombination of plasmids in a packaging cell.

As used herein, a transgene or therapeutic nucleic acid molecule includes DNA and RNA molecules encoding an RNA or polypeptide. Such molecules may be “native” or naturally-derived sequences; they may also be “non-native” or “foreign” that are naturally- or recombinantly-derived. The term “transgene,” which may be used interchangeably herein with the term “therapeutic nucleic acid molecule,” is often used to describe a heterologous or foreign (exogenous) gene that is carried by a viral vector and transduced into a host cell.

Therefore, therapeutic nucleotide nucleic acid molecules include antisense sequences or nucleotide sequences which may be transcribed into antisense sequences. Therapeutic nucleotide sequences (or transgenes) all include nucleic acid molecules that function to produce a desired effect in the cell or cell nucleus into which said therapeutic sequences are delivered. For example, a therapeutic nucleic acid molecule can include a sequence of nucleotides that encodes a functional protein intended for delivery into a cell which is unable to produce that functional protein.

As used herein, the vitreous of the eye refers to material that fills the chamber behind the lens of the eye (i.e., vitreous humor or vitreous body).

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences may be cis acting or may be responsive to trans acting factors. Promoters, depending upon the nature of the regulation, may be constitutive or regulated.

Thus, promoters are nucleic acid fragments that contain a DNA sequence that controls the expression of a gene located 3′ or downstream of the promoter. The promoter is the DNA sequence to which RNA polymerase specifically binds and initiates RNA synthesis (transcription) of that gene, typically located 3′ of the promoter. A promoter also includes DNA sequences that direct the initiation of transcription, including those to which RNA polymerase specifically binds. If more than one nucleic acid sequence encoding a particular polypeptide or protein is included in a therapeutic viral vector or nucleotide sequence, more than one promoter or enhancer element may be included, particularly if that would enhance efficiency of expression.

A regulatable or inducible promoter may be described as a promoter wherein the rate of RNA polymerase binding and initiation is modulated by external stimuli. (see, e.g., U.S. Pat. Nos. 5,750,396 and 5,998,205). Such stimuli include various compounds or compositions, light, heat, stress, chemical energy sources, and the like. Inducible, suppressible and repressible promoters are considered regulatable promoters.

Regulatable promoters may also include tissue-specific promoters. Tissue-specific promoters direct the expression of the gene to which they are operably linked to a specific cell type. Tissue-specific promoters cause the gene located 3′ of it to be expressed predominantly, if not exclusively, in the specific cells where the promoter expressed its endogenous gene. Typically, it appears that if a tissue-specific promoter expresses the gene located 3′ of it at all, then it is expressed appropriately in the correct cell types (see, e.g., Palmiter et al. (1986) Ann. Rev. Genet. 20: 465-499).

As used herein, the phrase “operatively linked” generally means the sequences or segments have been covalently joined into one piece of DNA, whether in single or double stranded form, whereby control sequences on one segment control expression or replication or other such control of other segments. The two segments are not necessarily contiguous.

As used herein, exogenous encompasses any therapeutic composition that is administered by the therapeutic methods provided herein. Thus, exogenous may also be referred to herein as foreign, or non-native or other equivalent expression.

B. Ad37 Fiber Tropism

The adenovirus fiber protein is a major determinant of adenovirus tropism (Gall et al. (1996) J. Virol. 70:2116-2123; Stevenson et al. (1995) J. Virol. 69:2850-2857). The fiber protein extends from the capsid and mediates viral binding to the cell surface by binding to specific cell receptors (Philipson et al. (1968) J. Virol. 2:1064-1075). The fiber is a trimeric protein that includes an N-terminal tail domain that interacts with the adenovirus penton base, a central shaft domain of varying length, and a C-terminal knob domain that contains the cell receptor binding site (Chroboczek et al. (1995) Curr. Top.Microbiol.Immunol. 199:163-200; Riurok et al. (1990) J. Mol. Biol. 215:589-596; Stevenson et al. (1995) J. Virol. 69:2850-2857). Fiber proteins of most adenovirus subgroups have been shown to bind specifically or selectively to the 46 kDa coxsackievirus-adenovirus receptor (CAR), (Bergelson et al. (1997) Science 275:1320-1323; Roelvink et al. (1998) J. Virol. 72:7909-7915). CAR appears to be expressed in a variety of human tissues, including the lung, at various levels (Bergelson et al. (1997) Science 275:1320-1323), but Ad37 binds poorly to lung epithelial cells (Huang et al. (1999) J. Virol. 73:2798-2802). This suggests that the tropism of this serotype may be influenced by factors independent of CAR expression.

Structural and biochemical data also suggest that distinct receptor binding sites are located on different regions of the Ad5 and Ad37 fiber knobs. Adopting the nomenclature of Xia et al. (Xia et al. (1994) Structure 2:1259-1270), the receptor binding site for Ad5 is located at the AB-loop on the side of the fiber knob (Bewley et al. (1999) Science 286:1579-1583; Roelvink et al. (1999) Science 286:1568-1571). It is known that a lysine residue at position 240 of the Ad37 fiber, located in the CD-loop, is important for receptor binding (Huang et al. (1999) J. Virol. 73:2798-2802). The co-crystal structure of the Ad12 knob and the N-terminal domain of CAR (Bewley et al. (1999) Science 286:1579-1583) show that the CD-loop does not contact CAR. It thus appears that different regions of the Ad5 and Ad37 fiber knobs recognize distinct cell receptors.

A 46 kDa receptor for coxsackieviruses and adenoviruses (CAR) mediates attachment for many adenovirus serotypes. The wide distribution of CAR fails to explain why certain adenovirus serotypes (i.e. Ad37) are highly associated with severe ocular infections such as epidemic keratoconjunctivitis (EKC). Ad37 does not use CAR, but instead uses a glycoprotein that contains sialic acid as its primary receptor (Arnberg et al. ((2000) J. Virol. 74:42-48). The modest number of Ad37 binding sites per cell (Huang et al. (1999) J. Virol. 73:2798-2802) also suggests that Ad37 recognizes a specific glycoprotein as its primary receptor for binding to conjunctival cells.

Adenovirus type 37 (subgroup D) has been associated with infections of the eye and genital tract. The tropism of Ad37 derives from the binding preference of its fiber protein, which binds to a receptor located on the surface of cells including Chang C, conjunctival epithelial cell line (Huang et al. (1999) J. Virology 73:2798-2802).

A protein receptor that is preferentially expressed on conjunctival cells to which Ad37 fiber binds is shown herein. The preferential expression of the Ad37 receptor protein on conjunctival cells suggests that this receptor likely influences Ad37 tropism and should play a key role in ocular pathogenesis. It is shown herein that Ad37 uses a distinct protein receptor that is selectively expressed on conjunctival cells. It is shown that Ad37 binds well to conjunctival cells (Chang C), but poorly to lung carcinoma cells (A549). To determine if infection correlated with cell binding, an Ad5 vector containing the Ad37 fiber protein was constructed. The ‘pseudotyped’ vector delivered transgenes to Chang C cells better than to A549 cells. Ad37 binding was abolished by protease treatment of Chang C cells, indicating the receptor is a membrane protein. Ad37 binding to conjunctival cells is shown herein to be calcium-dependent. It is also shown that Ad37 infection was not inhibited by a function-blocking anti-CAR monoclonal antibody, which is a feature distinct from Ad5 fiber interaction with CAR. Using a virus overlay protein blot assay (VOPBA), calcium-dependent Ad37 binding to a 50 KDa membrane protein on Chang C cells, but not A549 cells was detected. Ad19p a closely related serotype that fails to bind to conjunctival cells, does not recognize the 50 kDa protein. Together, these data indicate that the 50 kDa protein is a candidate receptor for Ad37 on conjunctival cells.

Significantly, it is also shown herein that, upon administration of the vector to the vitreous humor, the recombinant adenovirus with the Ad37 fiber preferentially and selectively binds to photoreceptor cells. Hence, a recombinant adenoviral delivery vehicle that has an Ad37 fiber protein can serve as a vector for delivery of therapeutic agents to the eye for treatment of ocular disorders, including genetic and acquired disorders. The identification of the receptor for Ad37 and the resulting recognition of Ad37 tropism allows targeting of adenovirus vectors to specific human ocular cells.

As noted, fiber plays a crucial role in adenovirus infection by attaching the virus to a specific receptor on a cell surface. Hexon, penton and fiber capsomeres are the major components on the surface of the virion. The fiber is an elongated protein which exists as a trimer of three identical polypeptides (polypeptide IV) of 582 amino acids in length. An adenovirus fiber includes three domains: an N-terminal tail domain that interacts with penton base; a shaft composed of variable numbers of repeats of a 15-amino-acid segment that forms beta-sheet and beta-bends; and a knob at the C-terminus (“head domain”) that contains the type-specific antigen and is responsible for binding to the cell surface receptor. The gene encoding the fiber protein from Ad2 has been expressed in human cells and has been shown to be correctly assembled into trimers, glycosylated and transported to the nucleus (see, e.g., Hong and Engler, Virology 185: 758-761, 1991). Thus, alteration of the fiber in recombinant Ad vectors can lead to alteration in gene delivery.

As shown herein, alteration of fiber in recombinant Ad vectors such that the fiber is derived from Ad37 or another adenovirus serotype D, provides a means for selective delivery of a recombinant virus to particular cells in the eye, including conjunctival cells, and most significantly photoreceptors, thereby providing a means for targeted delivery to photoreceptor cells.

Photoreceptor cells are implicated in a number of hereditary and acquired retinal degenerative disorders. In addition, photoreceptor cells are located such that products produced therein can be delivered to other areas of the eye by virtue of the blood flow in the vicinity of the photoreceptor cells and also by virtue of the proximity of the photoreceptors to the retinal pigmented epithelium (RPE) and other retinal cells.

Hence it is contemplated herein that the recombinant viral vector will include a packaged recombinant adenovirus genome containing at least the minimal elements for replication and packaging; heterologous DNA encoding a desired gene product, typically a therapeutic product or plurality of products, such as several trophic factors, whose combined activity is effective for treating a disorder, such as a retinal degenerative disorder; and the resulting virion particles will include a fiber that has a sufficient portion to confer specific targeting to photoreceptor cells when the recombinant viral particles are introduced into the aqueous humor of a mammalian, preferably a human, eye, or otherwise contacted with the photoreceptor cells. The fiber may be a chimeric protein that has been modified for effective interaction with other coat structural proteins, such as penton. In addition, the fiber may be modified to include other elements that alter its tropism to permit binding to other cells as well (see, e.g., U.S. Pat. Nos. 5,756,086 and 5,543,328, International PCT application No. WO 95/26412 and WO 98/44121 and Krasnykh, et al. (J. Virol. 70: 6839-46, 1996).

C. Construction of the Viral Particles

1. Selection of Viral Genome and Fiber Protein

Methods for preparing recombinant adenoviral vectors for gene product delivery are well known. Preferred among those are the methods exemplified herein (see EXAMPLES) and also described in copending U.S. application Ser. No. 09/482,682 (also filed as International PCT application No. PCT/US00/00265, filed Jan. 14, 2000, which claims priority to U.S. provisional application Ser. No. 60/115,920, as does U.S. application Ser. No. 09/482,682)).

As noted, any desired recombinant adenovirus is contemplated for use in the methods herein as long as the viral genome is packaged in a capsid that includes at least the portion of a fiber protein that provides selective binding to photoreceptor cells. This fiber protein is preferably from an adenovirus type D serotype and is preferably an Ad37 fiber. The fiber protein should retain the knob region at the C-terminus (“head domain”) from the Ad virus of subgroup D that contains the type-specific antigen and is responsible for binding to the cell surface receptor. Hence the fiber protein can be a chimeric fiber protein as long as it retains a sufficient portion of the type D serotype to specifically or selectively bind to photoreceptor cells. Generally the portion retained will be all or a portion of the knob region. The precise amount of knob region required can be determined empirically by including portions thereof and identifying the minimum residues from and Ad type D serotype, preferably Ad37, to effect selective targeting of a virion packaged with such fiber to photoreceptors in the eye upon introduction of the packaged virion into the aqueous humor.

Recombinant adenovirus containing heterologous nucleic acids that encode a desired product, such a gene to correct a genetic defect, may be made by any methods known to those of skill in the art. The viruses must be packaged in a cell line that results in expression of fiber on the particles that specifically, electively or preferentially targets (binds and results in internalization) the viral particle to cells in the eye. The fiber protein from Ad37 and other Adenoviruses of serotype D that infect the eye effects such targeting. The resulting adenovirus particles that express such fiber is administered by intraocular injection, subretinal injection, particularly intravitreal injection, or any means that results in preferential accumulation in photoreceptor cells.

The family of Adenoviridae includes many members with at least 47 known serotypes of human adenovirus (Ad1-Ad47) (Shenk, Virology, Chapter 67, in Fields et al., eds. Lippincott-Raven, Philadelphia, 1996,) as well as members of the genus Mastadenovirus including human, simian, bovine, equine, porcine, ovine, canine and opossum viruses and members of the Aviadenovirus genus, including bird viruses, such as CELO.

Thus it is contemplated that the methods herein can be applied to any recombinant viral vectors derived from any adenovirus species. One of skill in the art would have knowledge of the different adenoviruses (see, e.g.,Shenk, Virology, Chapter 67, in Fields et al., eds. Lippincott-Raven, Philadelphia, 1996,) and can construct recombinant viruses containing portions of the genome of any such virus.

In the exemplified embodiment, viral particles with Ad37 fiber were prepared. Site-directed mutations were made to the Ad37 fiber gene to make the tail sequence more closely match that of Ad5 to facilitate Ad37 fiber binding to the Ad5 penton base. The plasmid for the expression of the Ad37 fiber protein, pDV80, contains the CMV promoter, the adenovirus type 5 tripartite leader (TPL), and the modified Ad37 fiber gene sequence. Genes of interest, such as nucleic acid encoding the, β subunit of cGMP phosphodiesterase (βPDE), β-glucuronidase, rhodopsin, growth factors, anti-cancer agents, growth factor receptors and other anti-angiogenic agents, and anti-apoptotic agents, can be incorporated into these vectors using the methods known to those of skill in the art and exemplified herein.

Known adenovirus vectors, previously constructed for intraocular therapy (see, e.g., Bennett et al. (1996) Nature Medicine 2:649-654, which provides an Ad virus encoding βPDE for treatment of retinitis pigmentosa; Cayouette et al. (1998) Human Gene Therapy 8:423-430, which provides an Ad vector that expresses CNTF for treatment of retinitis pigmentosa and other retinal degenerative diseases; and Li et al. (1995) Proc. Natl. Acad. Sci. U.S.A. 92:7700-7704, which provides an Ad virus vector that encodes a human β-glucuronidase for treatment of lysosomal storage disease caused by β-glucuronidase deficiency) can be modified by repackaging the recombinant genome using a packaging line that expresses an Ad37 fiber or other D serotype fiber.

For exemplification, nucleic acid encoding GFP was incorporated into these vectors as a means to visualize their localization. Other genes, such as genes that encode therapeutic products, my be included in place of or in addition to GFP.

Plasmid pDV80 was electroporated into E1-2a S8 cells and stable lines were selected. The fiber-deleted vectors Ad5.βgal.ΔF and Ad5.GFP.ΔF were grown in cells in a resulting cell line, designated 705, to produce virions, which express the Ad37 fiber (Ad5.βgal.ΔF/37F and Ad5.GFP.ΔF/37F) and CsCl-purified. These virions selectively transduce photoreceptor cells when injected intraocularly into the vitreous humor.

2. Packaging

Recombinant adenoviral vectors generally have at least a deletion in the first viral early gene region, referred to as E1, which includes the E1a and E1b regions. Deletion of the viral E1 region renders the recombinant adenovirus defective for replication and incapable of producing infectious viral particles in subsequently-infected target cells. Thus, to generate E1-deleted adenovirus genome replication and to produce virus particles requires a system of complementation which provides the missing E1 gene product. E1 complementation is typically provided by a cell line expressing E1, such as the human embryonic kidney packaging cell line, i.e. an epithelial cell line, called 293. Cell line 293 contains the E1 region of adenovirus, which provides E1 gene region products to “support” the growth of E1-deleted virus in the cell line (see, e.g., Graham et al., J. Gen. Virol. 36: 59-71, 1977). Additionally, cell lines that may be usable for production of defective adenovirus having a portion of the adenovirus E4 region have been reported (WO 96/22378).

Multiply deficient adenoviral vectors and complementing cell lines have also been described (WO 95/34671, U.S. Pat. No. 5,994,106).

Copending U.S. application Ser. No. 09/482,682 (also filed as International PCT application No. PCT/US00/00265, filed Jan. 14, 2000)) provides packaging cell lines that support viral vectors with deletions of major portions of the viral genome, without the need for helper viruses and also provides cell lines and helper viruses for use with helper-dependent vectors. The packaging cell line has heterologous DNA stably integrated into the chromosomes of the cellular genome. The heterologous DNA sequence encodes one or more adenovirus regulatory and/or structural polypeptides that complement the genes deleted or mutated in the adenovirus vector genome to be replicated and packaged. The packaging cell line express, for example, one or more adenovirus structural proteins, polypeptides, or fragments thereof, such as penton base, hexon, fiber, polypeptide IIIa, polypeptide V, polypeptide VI, polypeptide VII, polypeptide VIII, and biologically active fragments thereof. The expression can be constitutive or under the control of a regulatable promoter. These cell lines are designed for expression of recombinant adenoviruses intended for delivery of therapeutic products.

Particular packaging cell lines complement viral vectors having a deletion or mutation of a DNA sequence encoding an adenovirus structural protein, regulatory polypeptides E1A and E1B, and/or one or more of the following regulatory proteins or polypeptides: E2A, E2B, E3, E4, L4, or fragments thereof.

The packaging cell lines are produced by introducing each DNA molecule into the cells and then into the genome via a separate complementing plasmid or plurality of DNA molecules encoding the complementing proteins can be introduced via a single complementing plasmid. Of interest herein, is a variation in which the complementing plasmid includes DNA encoding adenovirus fiber protein (or a chimeric or modified variant thereof), from Ad virus of subgroup D, such as Ad37, polypeptide or fragment thereof.

For therapeutic applications, the delivery plasmid further includes a nucleotide sequence encoding a foreign polypeptide. Exemplary delivery plasmids include, but are not limited to, pDV44, pΔE1Bβ-gal and pΔE1sp1B. In a similar or analogous manner, therapeutic genes may be introduced.

The cell further includes a complementing plasmid encoding a fiber as contemplated herein; the plasmid or portion thereof is integrated into a chromosome(s) of the cellular genome of the cell.

In one embodiment, a composition comprises a cell containing first and second delivery plasmids wherein a first delivery plasmid comprises an adenovirus genome lacking a nucleotide sequence encoding fiber and incapable of directing the packaging of new viral particles in the absence of a second delivery plasmid, and a second delivery plasmid comprises an adenoviral genome capable of directing the packaging of new viral particles in the presence of the first delivery plasmid.

In a variation, the packaging cell line expresses fiber protein or chimeric variant thereof from an Ad virus of subgroup D, preferably Ad37, serotype or it can be any fiber protein but one that has been modified to include the portion of the Ad virus of subgroup D, such as Ad37, responsible for selective targeting to photoreceptors upon introduction into the vitreous humor of the eye of a mammal, preferably a human. The fiber protein can be further modified to include a non-native amino acid residue sequence that targets additional specific receptors. In all instances, the modification should not disrupt trimer formation or transport of fiber into the nucleus. In another variation, the non-native amino acid residue sequence alters the binding specificity of the fiber for a targeted cell type. The structural protein is fiber can include amino acid residue sequences from more than one adenovirus serotype. The nucleotide sequences encoding fiber protein or polypeptide need not be modified solely at one or both termini; fiber protein, may be modified “internally” as well as at the termini.

Additional nucleic acid fragments can encode polypeptides that are added to the fiber protein. In one variation, the non-native amino acid residue sequence is coupled to the carboxyl terminus of the fiber. In another, the non-native amino acid residue sequence further includes a linker sequence. Alternatively, the fiber protein further comprises a ligand coupled to the linker. Suitable ligands include, but are not limited to, ligands that specifically or selectively bind to a cell surface receptor and ligands that can be used to couple other proteins or nucleic acid molecules. Typically, the packaging cell lines will contain nucleic acid encoding the fiber protein or modified protein stably integrated into a chromosome or chromosomes in the cellular genome.

The packaging cell line can be derived from a procaryotic cell line or from a eukaryotic cell line. While various embodiments suggest the use of mammalian cells, and more particularly, epithelial cell lines, a variety of other, non-epithelial cell lines are used in various embodiments. Thus, while various embodiments disclose the use of a cell line selected from among the 293, A549, W162, HeLa, Vero, 211, and 211A cell lines, and any other cell lines suitable for such use are likewise contemplated herein.

3. Components of the Nucleic Acid Molecule Included in the Particle

A recombinant viral vector or therapeutic viral vector for use in the methods herein, typically includes a nucleic acid fragment that encodes a protein or polypeptide molecule, or a biologically active fragment thereof, or other regulatory sequence, that is intended for use in therapeutic applications.

The nucleic acid molecule to be packaged in the viral particle also may include an enhancer element and/or a promoter located 3′ or 5′ to and controlling the expression of the therapeutic product-encoding nucleic acid molecule if the product is a protein. Further, for purposes herein, the promoter and/or other transcriptional and translational regulatory sequences controlling expression of the product is preferably one that is expressed specifically in the targeted cells, such as the a photoreceptor-specific promoter, such as a rhodopsin gene promoter.

The nucleic acid molecule to be packaged in viral capsid includes at least 2 different operatively linked DNA segments. The DNA can be manipulated and amplified by PCR as described herein and by using standard techniques, such as those described in Molecular Cloning: A Laboratory Manual, 2nd Ed., Sambrook et al., eds., Cold Spring Harbor, New York (1989). Typically, to produce such molecule, the sequence encoding the selected polypeptide and the promoter or enhancer are operatively linked to a DNA molecule capable of autonomous replication in a cell either in vivo or in vitro. By operatively linking the enhancer element or promoter and nucleic acid molecule to the vector, the attached segments are replicated along with the vector sequences.

Thus, the recombinant DNA molecule (rDNA) is a hybrid DNA molecule comprising at least 2 nucleotide sequences not normally found together in nature. In various preferred embodiments, one of the sequences is a sequence encoding an Ad-derived polypeptide, protein, or fragment thereof. The nucleic acid molecule intended to be packaged is from about 20 base pairs to about 40,000 base pairs in length, preferably about 50 bp to about 38,000 bp in length. In various embodiments, the nucleic acid molecule is of sufficient length to encode one or more adenovirus proteins or functional polypeptide portions thereof. Since individual Ad polypeptides vary in length from about 19 amino acid residues to about 967 amino acid residues, encoding nucleic acid molecules from about 50 bp up to about 3000 bp, depending on the number and size of individual polypeptide-encoding sequences that are “replaced” in the viral vectors by therapeutic product-encoding nucleic acid molecules.

Preferably the molecule includes an adenovirus tripartite leader (TPL) nucleic acid sequence operatively linked to an intron containing RNA processing signals (such as for example, splice donor or splice acceptor sites) suitable for expression in the packaging cell line. Most preferably the intron contains a splice donor site and a splice acceptor site. Alternatively, the TPL nucleotide sequence may not comprise an intron. The intron includes any sequence of nucleotides that function in the packaging cell line to provide RNA processing signals, including splicing signals. Introns have been well characterized from a large number of structural genes, and include but are not limited to a native intron 1 from adenovirus, such as Ad5's TPL intron 1; others include the SV40 VP intron; the rabbit beta-globin intron, and synthetic intron constructs (see, e.g., Petitclerc et al. (1995)J. Biothechnol., 40:169; and Choi et al. (19910 Mol. Cell. Biol., 11:3070).

The nucleic acid molecule encoding the TPL includes either (a) first and second TPL exons or (b) first, second and third TPL exons, where each TPL exon in the sequence is selected from among the complete TPL exon 1, partial TPL exon 1, complete TPL exon 2 and complete TPL exon 3. A complete exon is one which contains the complete nucleic acid sequence based on the sequence found in the wild type viral genome. Preferably the TPL exons are from Ad2, Ad3, Ad5, Ad7 and the like, however, they may come from any Ad serotype, as described herein. A preferred partial TPL exon 1 is described in the Examples. The use of a TPL with a partial exon 1 has been reported (International PCT application No. WO 98/13499).

The intron and the TPL exons can be operatively linked in a variety of configurations to provide a functional TPL nucleotide sequence. An intron may not be a part of the construct. For example, the intron can be positioned between any of TPL exons 1, 2 or 3, and the exons can be in any order of first and second, or first/second/third. The intron can also be placed preceding the first TPL exon or following the last TPL exon. In a preferred embodiment, complete TPL exon 1 is operatively linked to complete TPL exon 2 operatively linked to complete TPL exon 3. In a preferred variation, adenovirus TPL intron 1 is positioned between complete TPL exon 1 and complete TPL exon 2. It may also be possible to use analogous translational regulators from other viral systems such as rabiesvirus.

A preferred “complete” TPL nucleic acid molecule containing complete TPL exons 1, 2 and 3 with adenovirus intron 1 inserted between exons 1 and 2 has a nucleotide sequence shown in SEQ ID NO: 32. A preferred “partial” TPL nucleic acid molecule containing partial TPL exon 1 and complete TPL exons 2 and 3 in that order has a nucleotide sequence shown in SEQ ID NO: 26. The construction of these preferred TPL nucleotide sequences is described in the Examples.

Thus, preferred expression cassettes and complementing plasmids for expressing adenovirus structural genes, particularly fiber protein, contain an adenovirus TPL nucleotide sequence as described herein.

4. Complementing Plasmids

Also contemplated are the use of nucleic acid molecules, typically in the form of DNA plasmid vectors, which are capable of expression of an adenovirus structural protein or regulatory protein. Because these expression plasmids are used to complement the defective genes of a recombinant adenovirus vector genome, the plasmids are referred to as complementing or complementation plasmids.

The complementing plasmid contains an expression cassette, a nucleotide sequence capable of expressing a protein product encoded by the nucleic acid molecule. Expression cassettes typically contain a promoter and a structural gene operatively linked to the promoter. The complementing plasmid can further include a sequence of nucleotides encoding TPL nucleotide to enhance expression of the structural gene product when used in the context of adenovirus genome replication and packaging.

A complementing plasmid can include a promoter operatively linked to a sequence of nucleotides encoding an adenovirus structural polypeptide, such as, but are not limited to, penton base; hexon; fiber; polypeptide IIIa; polypeptide V; polypeptide VI; polypeptide VII; polypeptide VIII; and biologically active fragments thereof. In another variation, a complementing plasmid may also include a sequence of nucleotides encoding a first adenovirus regulatory polypeptide, a second regulatory polypeptide, and/or a third regulatory polypeptide, and any combination of the foregoing.

Plasmid pDV80 is a preferred plasmid herein. Other plasmids constructed in an analogous manner to encode modified fiber proteins and chimeric fiber proteins are also contemplated herein.

5. Nucleic Acid Molecule Synthesis

A nucleic acid molecule comprising synthetic oligonucleotides can be prepared using any suitable method, such as the phosphotriester or phosphodiester methods (see, e.g., Narang (1979) et al., Meth. Enzymol., 68:90; U.S. Pat. No. 4,356,270; and Brown et al., (1979) Meth. Enzymol., 68:109). For oligonucleotides, the synthesis of the family members can be conducted simultaneously in a single reaction vessel, or can be synthesized independently and later admixed in preselected molar ratios. For simultaneous synthesis, the nucleotide residues that are conserved at preselected positions of the sequence of the family member can be introduced in a chemical synthesis protocol simultaneously to the variants by the addition of a single preselected nucleotide precursor to the solid phase oligonucleotide reaction admixture when that position number of the oligonucleotide is being chemically added to the growing oligonucleotide polymer. The addition of nucleotide residues to those positions in the sequence that vary can be introduced simultaneously by the addition of amounts, preferably equimolar amounts, of multiple preselected nucleotide precursors to the solid phase oligonucleotide reaction admixture during chemical synthesis. For example, where all four possible natural nucleotides (A,T,G and C) are to be added at a preselected position, their precursors are added to the oligonucleotide synthesis reaction at that step to simultaneously form four variants (see, e.g., Ausubel et al. (Current Protocols in Molecular Biology, Suppl. 8. p.2.11.7, John Wiley & Sons, Inc., New York, 1991).

Nucleotide bases other than the common four nucleotides (A,T,G or C), or the RNA equivalent nucleotide uracil (U), can also be used. For example, it is well known that inosine (I) is capable of hybridizing with A, T and G, but not C. Examples of other useful nucleotide analogs are known in the art and may be found referred to in 37 C.F.R. § 1.822.

Thus, where all four common nucleotides are to occupy a single position of a family of oligonucleotides, that is, where the preselected nucleotide sequence is designed to contain oligonucleotides that can hybridize to four sequences that vary at one position, several different oligonucleotide structures are contemplated. The composition can contain four members, where a preselected position contains A,T,G or C. Alternatively, a composition can contain two nucleotide sequence members, where a preselected position contains I or C, and has the capacity to hybridize at that position to all four possible common nucleotides. Finally, other nucleotides may be included at the preselected position that have the capacity to hybridize in a non-destabilizing manner with more than one of the common nucleotides in a manner similar to inosine.

Similarly, larger nucleic acid molecules can be constructed in synthetic oligonucleotide pieces, and assembled by complementary hybridization and ligation, as is well known.

D. Adenovirus Expression Vector Systems

The adenovirus vector genome that is encapsulated in the virus particle and that expresses exogenous genes in a gene therapy setting is a key component of the system. Thus, the components of a recombinant adenovirus vector genome include the ability to express selected adenovirus structural genes, to express a desired exogenous protein, and to contain sufficient replication and packaging signals that the genome is packaged into a gene delivery vector particle. The preferred replication signal is an adenovirus inverted terminal repeat containing an adenovirus origin of replication, as is well known and described herein.

Although adenovirus include many proteins, not all adenovirus proteins are required for assembly of a recombinant adenovirus particle (vector). Thus, deletion of the appropriate genes from a recombinant Ad vector permits accommodation of even larger “foreign” DNA segments.

A preferred recombinant adenovirus vector genome is “helper independent” so that genome can replicate and be packaged without the help of a second, complementing helper virus. Complementation is provided by a packaging cell.

In a preferred embodiment, the adenovirus vector genome does not encode a functional adenovirus fiber protein. A non-functional fiber gene refers to a deletion, mutation or other modification to the adenovirus fiber gene such that the gene does not express any or insufficient adenovirus fiber protein to package a fiber-containing adenovirus particle without complementation of the fiber gene by a complementing plasmid or packaging cell line. Such a genome is referred to as a “fiberless” genome, not to be confused with a fiberless particle. Alternatively, a fiber protein may be encoded but is insufficiently expressed to result in a fiber containing particle.

Thus, contemplated for use are helper-independent fiberless recombinant adenovirus vector genomes that include genes that (a) express all adenovirus structural gene products but express insufficient adenovirus fiber protein to package a fiber-containing adenovirus particle without complementation of said fiber gene, (b) express an exogenous protein, and (c) contain an adenovirus packaging signal and inverted terminal repeats containing adenovirus origin of replication.

The adenovirus vector genome is propagated in the laboratory in the form of rDNA plasmids containing the genome, and upon introduction into an appropriate host, the viral genetic elements provide for viral genome replication and packaging rather than plasmid-based propagation. Exemplary methods for preparing an Ad-vector genome are described in the Examples.

A vector herein includes a nucleic acid (preferably DNA) molecule capable of autonomous replication in a cell and to which a DNA segment, e.g., a gene or polynucleotide, can be operatively linked to bring about replication of the attached segment. For purposes herein, one of the nucleotide segments to be operatively linked to vector sequences encodes at least a portion of a therapeutic nucleic acid molecule. As noted above, therapeutic nucleic acid molecules include those encoding proteins and also those that encode regulatory factors that can lead to expression or inhibition or alteration of expression of a gene product in a targeted cell.

1. Nucleic Acid Gene Expression Cassettes

In various embodiments, a peptide-coding sequence of the therapeutic gene is inserted into an expression vector and expressed; however, it is also feasible to construct an expression vector which also includes some non-coding sequences as well. Preferably, however, non-coding sequences are excluded. Alternatively, a nucleotide sequence for a soluble form of a polypeptide may be utilized. Another preferred therapeutic viral vector includes a nucleotide sequence encoding at least a portion of a therapeutic nucleotide sequence operatively linked to the expression vector for expression of the coding sequence in the therapeutic nucleotide sequence.

The choice of viral vector into which a therapeutic nucleic acid molecule is operatively linked depends directly, as is well known in the art, on the functional properties desired, e.g., vector replication and protein expression, and the host cell to be transformed—these being limitations inherent in the art of constructing recombinant DNA molecules. Although certain adenovirus serotypes are recited herein in the form of specific examples, it should be understood that the use of any adenovirus serotype, including hybrids and derivatives thereof are contemplated.

A translatable nucleotide sequence is a linear series of nucleotides that provide an uninterrupted series of at least 8 codons that encode a polypeptide in one reading frame. Preferably, the nucleotide sequence is a DNA sequence. The vector itself may be of any suitable type, such as a viral vector (RNA or DNA), naked straight-chain or circular DNA, or a vesicle or envelope containing the nucleic acid material and any polypeptides that are to be inserted into the cell.

2. Promoters

As noted elsewhere herein, an expression nucleic acid in an Ad-derived vector may also include a promoter, particularly a tissue or cell specific promoter, preferably one expressed in ocular cells, particularly photoreceptors.

Promoters contemplaged for use herein include regulatable (inducible) as well as constitutive promoters, which may be used, either on separate vectors or on the same vector. Some useful regulatable promoters are those of the CREB-regulated gene family and include inhibin, gonadotropin, cytochrome c, glucagon, and the like. (See, e.g., International PCT application No. WO 96/14061). Preferably the promoter selected is from a photoreceptor-specific gene, such as a rhodopsin gene or gene that encodes a protein that regulates rhodopsin expression.

E. Formulation and Administration

Compositions containing therapeutically effective concentrations of recombinant adenovirus delivery vectors are provided. These are for delivery of therapeutic gene products to cells, particularly cells express a particular 50 kDa receptor or other receptor with which the vectors interact. These cells include cells of the eye and genital tract. Of particular interest are photoreceptor cells of the eye. Administration is effected by any means through which contacting with the photoreceptors is effected. Preferable modes of administration include, but are not limited to, subretinal injection, particularly intravitreal injection, to provide access to photoreceptor cells.

The recombinant viral compositions may also be formulated for implantation into the anterior or posterior chamber of the eye, preferably the vitreous cavity, in sustained released formulations, such as those adsorbed to biodegradable supports, including collagen sponges, or in liposomes. Sustained release formulations may be formulated for multiple dosage administration, so that during a selected period of time, such as a month or up to about a year, several dosages are administered. Thus, for example, liposomes may be prepared such that a total of about two to up to about five or more times the single dosage is administered in one injection.

The vectors are formulated in an ophthalmologically acceptable carrier for intraocular, preferably intravitreal, administration in a volume of between about 0.05 ml and 0.150 ml, preferably about 0.05 and 0.100 ml.

The composition can be provided in a sealed sterile vial containing an amount of a compound of formula 1, that upon intraocular administration will deliver a sufficient amount of viral particles to the photoreceptors in a volume of about 50 to 150 μl, containing at least about 10⁷, more preferably at least about 10⁸ plaque forming units in such volume. Typically, the vials will, thus, contain about 0.150 ml of the composition.

To prepare compositions the viral particles are dialzyed into a suitable ophthalmologically acceptable carrier or viral particles, for example, may be concentrated and/or mixed therewith. The resulting mixture may be a solution, suspension or emulsion. In addition, the viral particles may be formulated as the sole pharmaceutically active ingredient in the composition or may be combined with other active agents for the particular disorder treated.

For administration by intraocular injection or via eyedrops, suitable carriers include, but are not limited to, physiological saline, phosphate buffered saline (PBS), balanced salt solution (BSS), lactate Ringers solution, and solutions containing thickening and solubilizing agents, such as glucose, polyethylene glycol, and polypropylene glycol and mixtures thereof. Liposomal suspensions may also be suitable as pharmaceutically acceptable carriers. These may be prepared according to methods known to those skilled in the art. Suitable ophthalmologically acceptable carriers are known. Solutions or mixtures intended for ophthalmic use may be formulated as 0.01%-10% isotonic solutions, pH about 5-7, with appropriate salts [see, e.g., U.S. Pat. No. 5,116,868, which describes typical compositions of ophthalmic irrigation solutions and solutions for local application]. Such solutions, which have a pH adjusted to about 7.4, contain, for example, 90-100 mM sodium chloride, 4-6 mM dibasic potassium phosphate, 4-6 mM dibasic sodium phosphate, 8-12 mM sodium citrate, 0.5-1.5 mM magnesium chloride, 1.5-2.5 mM calcium chloride, 15-25 mM sodium acetate, 10-20 mM D.L.-sodium β-hydroxybutyrate and 5-5.5 mM glucose.

The compositions may be prepared with carriers that protect them from rapid elimination from the body, such as time release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and other types of implants that may be placed directly into the anterior or posterior chamber or vitreous cavity of the eye. The compositions may also be administered in pellets, such as Elvax pellets (ethylene-vinyl acetate copolymer resin).

Liposomal suspensions, including tissue-targeted liposomes, may also be suitable as pharmaceutically acceptable carriers. For example, liposome formulations may be prepared by methods known to those of skill in the art [see, e.g., Kimm et al. (1983) Bioch. Bioph. Acta 728:339-398; Assil et al. (1987) Arch Ophthalmol. 105:400; and U.S. Pat. No. 4,522,811]. The viral particles may be encapsulated into the aqueous phase of liposome systems.

The active materials can also be mixed with other active materials, that do not impair the desired action, or with materials that supplement the desired action or have other action, including viscoelastic materials, such as hyaluronic acid, which is sold under the trademark HEALON, which is a solution of a high molecular weight (MW) of about 3 millions fraction of sodium hyaluronate [manufactured by Pharmacia, Inc; see, e.g., U.S. Pat. Nos. 5,292,362, 5,282,851, 5,273,056, 5,229,127, 4,517,295 and 4,328,803], VISCOAT [fluorine-containing (meth)acrylates, such as, 1H,1H,2H,2H-heptadecafluorodecylmethacrylate; see, e.g., U.S. Pat. Nos. 5,278,126, 5,273,751 and 5,214,080; commercially available from Alcon Surgical, Inc.], ORCOLON [see, e.g., U.S. Pat. No. 5,273,056; commercially available from Optical Radiation Corporation], methylcellulose, methyl hyaluronate, polyacrylamide and polymethacrylamide [see, e.g., U.S. Pat. No. 5,273,751]. The viscoelastic materials are present generally in amounts ranging from about 0.5 to 5.0%, preferably 1 to 3% by weight of the conjugate material and serve to coat and protect the treated tissues. The compositions may also include a dye, such as methylene blue or other inert dye, so that the composition can be seen when injected into the eye. Additional active agents may be included.

The compositions can be enclosed in ampules, disposable syringes or multiple or single dose vials made of glass, plastic or other suitable material. Such enclosed compositions can be provided in kits. In particular, kits containing vials, ampules or other containers, preferably disposable vials with sufficient amount of the composition to deliver about 0.100 ml thereof, and disposable needles, preferably self sealing 25-30 gauge needles, are provided herein.

Finally, the compounds may be packaged as articles of manufacture containing packaging material, typically a vial, an ophthalmologically acceptable composition containing the viral particles and a label that indicates the therapeutic use of the composition.

Also provided are kits for practice of the methods herein. The kits contain one or more containers, such as sealed vials, with sufficient composition for single dosage administration, and one or more needles, such as self sealing 25-33 gauge needles, preferably 33 gauge or smaller needles, precisely calibrated syringes or other precisely calibrated delivery device, suitable for intravitreal injection.

Administration of the composition is preferably by intraocular injection, although other modes of administration may be effective, if the sufficient amount of the compound achieves contact with the vitreous cavity. Intraocular injection may be effected by intravitreal injection, aqueous humor injection or injection into the external layers of the eye, such as subconjunctival injection or subtenon injection, or by topical application to the cornea, if a penetrating formulation is used.

Administration

The compositions containing the compounds are administered intraocularly or by other means, such as topically in the form of penetrating eyedrops, whereby contact of the recombinant vectors with the aqueous humor is effected. Intraocular administration may be effected by intravitreal injection, aqueous humor injection, injection into the external layers of the eye, such as subconjunctival injection or subtenon injection, preferably in free form, but, alternatively, in liposomes or other sustained drug delivery device. Administration is preferably by intravitreal injection, preferably through self sealing 25-30 gauge needles or other suitably calibrated delivery device. Injection into the eye may be through the pars plana via the self-sealing needle.

It is further understood that, for any particular subject, specific dosage regimens should be adjusted over time according to the individual need and the professional judgment of the person administering or supervising the administration of the recombinant viruses, and that the concentration ranges set forth herein are exemplary only and are not intended to limit the scope or practice of the claimed methods

F. Diseases, Disorders and Therapeutic Products

1. Disease and Disorders

Retinitis Pigmentosa

Methods for specifically or selectively targeting recombinant adenovirus vectors for delivery of gene products, particularly therapeutic products are provided herein. These methods are particularly suitable for targeting cells that express receptors that are selectively recognized by Ad virus of subgroup D viruses, particularly Ad37. It is shown herein that these viruses selectively recognize receptors on cells, such as conjunctival cells and photoreceptors, that are not recognized by other adenoviruses. Hence, methods for targeting to these cell types by providing vectors that are packaged in viral particles that contain a sufficient portion of a fiber protein from one of these Ad serotypes to bind to these receptors. These methods are useful for targeting to photoreceptors and for treating ocular disorders, including, but are not limited to, inherited and acquired retinal, neovascular degenerative diseases (see table below).

It is estimated that 1 in 3,500 individuals in the United States suffer from one of the pigmented retinopathies. This group of retinal diseases, commonly called retinitis pigmentosa, is characterized by progressive loss of peripheral and night vision. Patients may be affected at almost any age and it is not uncommon to experience symptoms in early childhood in certain inherited forms. It has been shown that there are a variety of mutations in genes expressed in the photoreceptors, including genes in the rhodopsin gene and pathway that appear to be responsible for these diseases. In addition to mutations in rhodopsin, changes in the retinal pigmented epithelial (RPE) cells, also undergo degenerative changes and can form clumps of pigment that give rise to the characteristic pigmentary changes seen in patients with RP.

Angiogenesis and Ocular Diseases and Disorders

The vast majority of diseases that cause catastrophic loss of vision do so as a result of ocular neovascularization; age related macular degeneration (ARMD) affects 12-15 million American over the age of 65 and causes visual loss in 10-15% of them as a direct effect of choroidal (sub-retinal) neovascularization. The leading cause of visual loss for Americans under the age of 65 is diabetes; 1 6 million individuals in the United States are diabetic and 40,000 per year suffer from ocular complications of the disease, which often are a result of retinal neovascularization. Laser photocoagulation has been effective in preventing severe visual loss in subgroups of high risk diabetic patients, but the overall 10 year incidence of retinopathy remains essentially unchanged. For patients with choroidal neovascularization due to ARMD or inflammatory eye disease, such as ocular histoplasmosis, photocoagulation, with few exceptions, is ineffective in preventing visual loss. While recently developed, non-destructive photodynamic therapies hold promise for temporarily reducing individual loss in patients with previously untreatable choroidal neovascularization, only 61.4% of patients treated every 3-4 months had improved or stabilized vision compared to 45.9% of the placebo-treated group.

In the normal adult, angiogenesis is tightly regulated and limited to wound healing, pregnancy and uterine cycling. Angiogenesis is turned on by specific angiogenic molecules such as basic and acidic fibroblast growth factor (FGF), vascular endothelial growth factor (VEGF), angiogenin, transforming growth factor (TGF), tumor necrosis factor-α (TNF-α) and platelet derived growth factor (PDGF). Angiogenesis can be suppressed by inhibitory molecules such as interferon-α, thrombo-spondin-1, angiostatin and endostatin. It is the balance of these naturally occurring stimulators and inhibitors that controls the normally quiescent capillary vasculature. When this balance is upset, as in certain disease states, capillary endothelial cells are induced to proliferate, migrate and ultimately differentiate.

Angiogenesis plays a central role in a variety of diseases, including, but are not limited to, cancer and ocular neovascularization. Sustained growth and metastasis of a variety of tumors has also been shown to be dependent on the growth of new host blood vessels into the tumor in response to tumor derived angiogenic factors. Proliferation of new blood vessels in response to a variety of stimuli occurs as the dominant finding in the majority of eye diseases that blind, such as, but are not limited to, proliferative diabetic retinopathy (PDR), ARM D, rubeotic glaucoma, interstitial keratitis and retinopathy of prematurity. In these diseases, tissue damage can stimulate release of angiogenic factors resulting in capillary proliferation. VEGF plays a dominant role in iris neovascularization and neovascular retinopathies. While reports clearly show a correlation between intraocular VEGF levels and ischemic retinopathic ocular neovascularization, FGF likely plays a role. Basic and acidic FGF are known to be present in the normal adult retina, even though detectable levels are not consistently correlated with neovascularization. This may be largely due to the fact that FGF binds very tightly to charged components of the extracellular matrix and may not be readily available in a freely diffusible form that would be detected by standard assays of intraocular fluids.

A final common pathway in the angiogenic response involves integrin-mediated information exchange between a proliferating vascular endothelial cell and the extracellular matrix. This class of adhesion receptors, called integrins, are expressed as heterodimers having an α and β subunit on all cells. One such integrin, αᵥβ₃, is the most promiscuous member of this family and allows endothelial cells to interact with a wide variety of extracellular matrix components. Peptide and antibody antagonists of this integrin inhibit angiogenesis by selectively inducing apoptosis of the proliferating vascular endothelial cells. Two cytokine-dependent pathways of angiogenesis exist and may be defined by their dependency on distinct vascular cell integrins, αᵥβ₃ and αᵥβ₅. Specifically, basic FGF- and VEGF-induced angiogenesis depend on integrin αᵥβ₃ and αᵥβ₅, respectively, since antibody antagonists of each integrin selectively block one of these angiogenic pathways in the rabbit corneal and chick chorioallantoic membrane (CAM) models. Peptide antagonists that block all a, integrins inhibit FGF- and VEGF-stimulated angiogenesis. While normal human ocular blood vessels do not display either integrin, αᵥβ₃ and αᵥβ₅ integrins are selectively displayed on blood vessels in tissues from patients with active neovascular eye disease. While only αᵥβ₃ was consistently observed in tissue from patients with ARMD, αᵥβ₃ and αᵥβ₅ were present in tissues from patients with PDR. Systemically administered peptide antagonists of integrins blocked new blood vessel formation in a mouse model of retinal vasculogenesis.

In addition to adhesion events described above, cell migration through the extracellular matrix also depends on proteolysis. Matrix metalloproteinases are a family of zinc-requiring matrix-degrading enzymes that include the collagenases, gelatinases and stromelysins, all of which have been implicated in invasive cell behavior. Invasive cell processes such as tumor metastasis and angiogenesis have been found to be associated with the expression of integrins and MMP-2, MMP-2 are all found throughout the eye where they may interact to maintain a quiescent vasculature until the balance is upset, resulting in pathological angiogenesis. A non-catalytic C-terminal hemopexin-like domain of MMP-2 (PEX) can block cell surface collagenolytic activity and inhibit angiogenesis in the CAM model by preventing localization of MMP-2 to the surface of invasive cells through interaction with the integrin αᵥβ₃.

Hence, anti-angiogenic agents have a role in treating retinal degeneration to prevent the damaging effects of these trophic and growth factors. Angiogenic agents, also have a role in promoting desirable vascularization to retard retinal degeneration by enhancing blood flow to cells.

Members of adenovirus subgroup D, Ad8, 19A, and 37, are infectious agents that cause particularly severe cases of epidemic keratoconjunctivitis (EKC) (Arnberg et al. (1998) Virology 227:239-244; Curtis et al. (1998) J. Med. Microbiol. 47:91-94; Ritterband et al. (1998) Rev.Med. Virol. 8:187-201; and Takeuchi et al. (1999) J. Clin. Microbiol. 37:3392-3394). There is no effective treatment for this debilitating and contagious disease and EKC continues to be a problem in ophthalmology clinics worldwide (Curtis et al. (1998) J. Med. Microbiol. 47:91-94, Lukashok et al. (1998) Curr. Clin. Top.Infec.Dis. 18:286-304). Hence the vectors herein may be used for treating the disease.

TABLE 3
Candidate targets for ocular disease therapy
CANDIDATE TARGETS FOR OCULAR DISEASE THERAPY

| Disease | Candidate target(s) |
|---|---|
| Retinitis pigmentosa | Rhodopsin gene, and genes that |
| | regulate expression thereof |
| | rds/peripherin |
| Stargardt's disease | rim protein (ARC protein) |
| Choroideremia | rab geranylgeranyl transferase |
| | CHM, TCD, CHML* |
| Gyrate Atrophy | ornithine aminotransferase |
| Macular dystrophy | rds/peripherin |

*see, “MSR6-yeast homologue of the choroideraemia gene,” Nature Genetics 3: 193-4 (1993)

TABLE 4
Other Diseases
Exudative Choroidal Diseases

ICSC, fluorescein angiogram
ICSC with large serious detachment of RPE (retinal pigmented epithelium)
ICSC with bullous retinal detachment
Macular drusen, exudative, confluent
Drusen, sub-RPE choroidal neovascularization
Drusen, notched serous detachment of RPE
Drusen, notched serous and hemorrhagic detachment of RPE
Drusen, serous and hemorrhagic detachment of RPE and retina
Drusen, organized RPE detachment causing bullous retinal detachment
Drusen, geographic atrophy of RPE
Drusen, exudative and cuticular, vitelliform macular detachment
Drusen, cuticular, large vitelliform macular detachment
North Carolina dystrophy with macular staphyloma
North Carolina dystrophy with macular staphyloma
Angioid streaks, pseudoxanthoma elasticum (PXE), CNVM
Angioid streaks, PXE, large notched retinal detachment
Myopic degeneration, Foerster-Fuchs spot
Presumed ocular histoplasmosis syndrome (POHS)
Submacular bacterial abscess
Toxocara canis, subretinal granuloma
Serpiginous (geographic) choroiditis
Posterior scleritis
Harada's disease
Posterior sympathetic uveitis
Benign reactive lymphoid hyperplasia of uveal tract
Choroidal ruptures and CNVM
Cavernous hemangioma of choroid
Choroidal osteoma
Choroidal nevus, serous macular detachment
Choroidal nevus with CNVM
Diffuse sclerochoroidal melanocytic nevus
Choroidal melanoma with serous detachment of RPE
Metastatic lung carcinoma to choroid
Sub-RPE reticulum cell sarcoma
RPE tear, idiopathic choroidal neovascularization

Heredodystrophic Disorders Affecting RPE & Retina

Best's vitelliform macular dystrophy
Best's vitelliform macular dystrophy with CNVM
Best's vitelliform macular dystrophy, multiple lesions
Adult-onset vitelliform foveomacular dystrophy
Pattern dystrophy simulating fundus flavimaculatus
Stargardt's disease (fundus flavimaculatus)
Asteroid macular dystrophy
Sjögren-Larssen syndrome
Oguchi's disease, light-adapted state
Oguchi's disease, dark-adapted state
Fundus albipunctatus
Retinitis pigmentosa, cystoid macular edema
Crystalline tapetoretinal dystrophy
Choroideremia
Goldmann-Favre syndrome
Sex-linked juvenile retinoschisis
Perivenous retinitis pigmentosa

Retinal Vascular Disorders

Retinal arteriovenous aneurysm
Central retinal artery occlusion
Cilioretinal artery obstruction
Ischemic retinopathy in systemic lupus erythematosus
Ischemic retinopathy in scleroderma
Hemorrhagic detachment of internal limiting membrane, hypertensive
retinopathy
Acquired retinal arterial macroaneurysm
Cystoid macular edema, aphakic
Cystoid macular edema, nicotinic acid maculopathy
Congenital retinal telangiectasis
Acquired bilateral juxtafoveal telangiectasis
Acquired bilateral juxtafoveal obliterative telangiectasis
Diabetic optic neuropathy
X-ray radiation exudative retinopathy
Sickle cell SC disease, macular hemorrhage
Retinal arterial aneurysms, arteritis, neuroretinitis
Branch retinal vein obstruction (BRVO)
BRVO, exudative maculopathy
BRVO, optic disc new vessels, photocoagulation
Waldenström's macroglobulinemia

Inflammatory Diseases of the Retina and Choroid

Luetic retinal vasculitis
Focal Candida retinal abscess
Toxoplasmosis, atrophic chorioretinal scar
Toxoplasmosis retinitis and macular detachment
Toxoplasmosis scar, CNVM, macular detachment
Diffuse unilateral subacute neuroretinitis, small worm
Diffuse unilateral subacute neuroretinitis, large worm
Cytomegalic inclusion disease, papillitis
Acute posterior multifocal placoid pigment epitheliopathy
Acute macular neuroretinitis
Sarcoid retinitis
Sarcoid papillitis
Behcet's disease
Vitiliginous (bird-shot) chorioretinitis
Multifocal choroiditis and panveitis (pseudo-POHS)

Retinal and Pigment Epithelial Hamartomas

Congenital grouped albinotic RPE spots
Congenital hyperplasia of RPE
Combined RPE and retinal hamartoma, juxtapapillary
Combined RPE and retinal hamartoma, peripheral
Cystic astrocytoma, juxtapapillary
Astrocytoma, macula
Astrocytoma, juxtapapillary
Cavernous hemangioma of retina
Juxtapapillary sessile retinal capillary hemangioma
Juxtapapillary endophytic retinal capillary hemangioma

Other Tumors of the Choroid

Choroidal metastasis
Choroidal osteoma
Choroidal hemangioma
Miscellaneous uveal tumors

Intraocular Lymphoid Tumors

The leukemias and lymphomas

Tumors of the Vitreous

Non-Hodgkins (“reticulum cell”) lymphoma
Tumor involvement of the vitreous cavity

Macular Disease

Age-related macular degeneration -- atrophic form
Exudative age-related macular degeneration
Choroidal neovascular membrane in degenerative myopia
Central serous retinopathy
Macular hole
Macular dystrophies

Retinal Vascular Disease

Etiologic mechanisms in diabetic retinopathy
Background diabetic retinopathy
Proliferative diabetic retinopathy
Retinal arterial obstructive disease
Central retinal vein occlusion
Retinal branch vein occlusion
Pregnancy and retinal disease
Pregnancy-induced hypertension
Hypertension
The rheumatic disease
Parafoveal telangiectasis
Coats disease
Disseminated intravascular systemic coagulopathy and related
vasculopathies
Hemoglobinopathies
Retinopathy of prematurity
Acquired retinal macroaneurysms
Eales disease
Radiation retinopathy
The ocular ischemic syndrome

Inflammatory Disease

Ocular toxoplasmosis
Ocular toxocariasis
Ocular cysticercosis
Cytomegalovirus infections of the retina
Retinal and ophthalmologic manifestations of AIDS
Acute retinal necrosis syndrome
Endogenous fungal infections of the retina and choroid
Pars planitis
Syphilis and tuberculosis
Diffuse unilateral subacute neuroretinitis
Scleritis
Birdshot retinochoroidopathy
Punctate inner choroidopathy
Sarcoidosis
Acute multifocal placoid pigment epitheliopathy
Geographic helicoid peripapillary choroidopathy (GHPC): serpiginous
choroiditis
Sympathetic ophthalmia
Vogt-Koyanigi-Harada syndrome (uveomeningitic syndrome)
Ciliochoroidal (uveal) effusion
Reproduced from: Stereoscopic Atlas of Ocular Diseases Diagnosis and Treatment, 2nd Edition, J. Donald O. Gass, Vol. 1 & 2, C.V. Mosley Co. (1987); and Retina Vol. II, Editor, Stephen J. Ryan, Medical Retina, C.V. Moslay Co. (1989).

2. Therapeutic Products

Therapeutic products include but are not limited to, wild-type genes that are defective in ocular disorders, such as rhodopsin, or fragments thereof sufficient to correct the genetic defect, trophic factors, including growth factors, inhibitors and agonists of trophic factors, anti-apoptosis factors and other products described herein or known to those of skill in the art to be useful for treatment of disorders of the eye or that can be treated by a product expressed by a photoreceptor.

OCULAR GENE THERAPY STRATEGIES

| GENERAL DISEASE | EXAMPLES | STRATEGY |
|---|---|---|
| Hereditary retinal and | Retinitis pigmentosa | Growth factors (e.g., |
| macular degeneration | Stargardt's disease | GDNF) |
| | •Other macular | anti-apoptotic factors |
| | dystrophies | (e.g., bcl2 gene) |
| | | Stargardt Disease Gene |
| | | (ABCR)^{†} |
| Neovascular | Diabetes | Anti-angiogenesis |
| | Choroidal | factors |
| | neovascularization | |
| Anti-tumor | Retinoblastoma | Antiproliferant |
| Glaucoma | Nerve fiber layer | Neuroprotective agent |
| | atrophy | |

^{†}See Allikmets etal. (1997) Science 277: 1805-1807.

For example, for treatment of retinitis pigmentosa the adenovirus vector can deliver a wild-type rhodopsin gene or a growth factor or trophic factor, such as ciliary neurotrophic factor CNTF; for treatment of Stargardt's disease, the vector can deliver a wild type ABCR (also called STGD1) or a growth factor or anti-angiogenic agent; for diabetic retinopathies, retinal vascularization the vector can deliver growth factors, such as a TGF (TGFβ), to prevent degeneration.

The following examples are included for illustrative purposes only and are not intended to limit the scope of the invention.

### EXAMPLE 1

Preparation of Adenovirus Packaging Cell Lines

Cell lines that are commonly used for growing adenovirus are useful as host cells for the preparation of adenovirus packaging cell lines. Preferred cells include 293 cells, an adenovirus-transformed human embryonic kidney cell line obtained from the ATCC, having Accession Number CRL 1573; HeLa, a human epithelial carcinoma cell line (ATCC Accession Number CCL-2); A549, a human lung carcinoma cell line (ATCC Accession Number CCL 1889); and other epithelial-derived cell lines. As a result of the adenovirus transformation, the 293 cells contain the E1 early region regulatory gene. All cells were maintained in complete DMEM+10% fetal calf serum unless otherwise noted.

These cell lines allow the production and propagation of adenovirus-based gene delivery vectors that have deletions in preselected gene regions and that are obtained by cellular complementation of adenoviral genes. To provide the desired complementation of such deleted adenoviral genomes in order to generate a viral vector, plasmid vectors that contain preselected functional units have been designed. Such units include but are not limited to E1 early region, E4 and the viral fiber gene. The preparation of plasmids providing such complementation, thereby being “complementary plasmids or constructs,” that are stably inserted into host cell chromosomes are described below.

A. Preparation of an E4-Expressing Plasmid for Complementation of E4-Gene-Deleted Adenoviruses

The viral E4 regulatory region contains a single transcription unit that is alternately spliced to produce several different mRNA products. The E4-expressing plasmid prepared as described herein and used to transfect the 293 cell line contains the entire E4 transcription unit. A DNA fragment extending from 175 nucleotides upstream of the E4 transcription start site including the natural E4 promoter to 153 nucleotides downstream of the E4 polyadenylation signal including the natural E4 terminator signal, corresponding to nucleotides 32667-35780 of the adenovirus type 5 (hereinafter referred to as Ad5) genome as described in Chroboczek et al. (Virol, 186:280-285 (1992), GenBank Accession Number M73260), was amplified from Ad5 genomic DNA, obtained from the ATCC, via the polymerase chain reaction (PCR). Sequences of the primers used were 5′CGGTACACAGAATTCAGGAGACACAACTCC3′ (forward or 5′ primer referred to as E4L) (SEQ ID NO: 1) and 5′GCCTGGATCCGGGAAGTTACGTAACGTGGGAAAAC3′ (SEQ ID NO: 2) (backward or 3′ primer referred to as E4R). To facilitate cloning of the PCR fragment, these oligonucleotides were designed to create new sites for the restriction enzymes EcoRI and BamHI, respectively, as indicated with underlined nucleotides. DNA was amplified via PCR using 30 cycles of 92 C for 1 minute, 50 C for 1 minute, and 72 C for 3 minutes resulting in amplified full-length E4 gene products.

The amplified DNA E4 products were then digested with EcoRI and BamHI for cloning into the compatible sites of pBluescript/SK+ by standard techniques to create the plasmid pBS/E4. A 2603 base pair (bp) cassette including the herpes simplex virus thymidine kinase promoter, the hygromycin resistance gene, and the thymidine kinase polyadenylation signal was excised from the plasmid pMEP4 (Invitrogen, San Diego, Calif.) by digestion with Fspl followed by addition of BamHI linkers (5′CGCGGATCCGCG3′) (SEQ ID NO: 3) for subsequent digestion with BamHI to isolate the hygromycin-containing fragment.

The isolated BamHI-modified fragment was then cloned into the BamHI site of pBS/E4 containing the E4 region to create the plasmid pE4/Hygro containing 8710 bp. The pE4/Hygro plasmid has been deposited with the ATCC under accession number 97739. The complete nucleotide sequence of pE4/Hygro is set forth in SEQ ID NO: 4. Position number 1 of the linearized vector corresponds to approximately the middle portion of the pBS/SK+ backbone. The 5′ and 3′ ends of the E4 gene are located at respective nucleotide positions 3820 and 707 of SEQ ID NO: 4 while the 5′ and 3′ ends of the hygromycin insert are located at respective nucleotide positions 3830 and 6470. In the clone that was selected for use, the E4 and hygromycin resistance genes were divergently transcribed.

B. Preparation of a Fiber-Expressing Plasmid for Complementation of Fiber-Gene-Deleted Adenoviruses

To prepare a fiber-encoding construct, primers were designed to amplify the fiber coding region from Ad5 genomic DNA with the addition of unique BamHI and NotI sites at the 5′ and 3′ ends of the fragment, respectively. The Ad5 nucleotide sequence is available with the GenBank Accession Number M18369. The 5′ and 3′ primers had the respective nucleotide sequences of 5′ATGGGATCCAAGATGAAGCGCGCAAGACCG3′ (SEQ ID NO: 5) and 5′CATAACGCGGCCGCTTCTTTATTCTTGGGC3′ (SEQ ID NO: 6), where the inserted BamHI and NotI sites are indicated by underlining. The 5′ primer also contained a nucleotide substitution 3 nucleotides 5′ of the second ATG codon (C to A) that is the initiation site. The nucleotide substitution was included so as to improve the consensus for initiation of fiber protein translation.

The amplified DNA fragment was inserted into the BamHI and NotI sites of pcDNA3 (Invitrogen) to create the plasmid designated pCDNA3/Fiber having 7148 bp. The parent plasmid contained the CMV promoter, the bovine growth hormone (BHG) terminator and the gene for conferring neomycin resistance. The viral sequence included in this construct corresponds to nucleotides 31040-32791 of the Ad5 genome.

The complete nucleotide sequence of pCDNA3/Fiber is listed in SEQ ID NO: 7 where the nucleotide position 1 corresponds to approximately the middle of the pcDNA3 vector sequence. The 5′ and 3′ ends of the fiber gene are located at respective nucleotide positions 916 with ATG and 2661 with TAA.

To enhance expression of fiber protein by the constitutive CMV promoter provided by the pcDNA vector, a BgIII fragment containing the tripartite leader (TPL) of adenovirus type 5 was excised from pRD112a (Sheay et al., BioTechniques, 15:856-862 (1993) and inserted into the BamHI site of pCDNA3/Fiber to create the plasmid pCLF having 7469 bp. The adenovirus tripartite leader sequence, present at the 5′ end of all major late adenoviral mRNAs as described by Logan et al., Proc._{—}Natl. Acad. Sci., USA, 81:3655-3659 (1984) and Berkner, BioTechniques, 6:61 6-629 (1988), also referred to as a “partial TPL” since it contains a partial exon 1, shows correspondence with the Ad5 leader sequence having three spatially separated exons corresponding to nucleotide positions 6081-6089 (the 3′ end of the first leader segment), 7111-7182 (the entire second leader segment), and 9644-9845 (the third leader segment and sequence downstream of that segment). The corresponding cDNA sequence of the partial tripartite leader sequence present in pCLF is included in SEQ ID NO: 8 bordered by BamHI/BgIII 5′ and 3′ sites at respective nucleotide positions 907-912 to 1228-1233. The nucleotide sequence of an isolated partial TPL is also listed separately as SEQ ID No. 22 with the noted 5′ and 3′ restriction sites and with the following nucleotide regions identified: 1-6 nt BgIII site; 1-18 nt polylinker; 19-27 nt last 9 nt of the first leader segment (exon 1); 28-99 nt second leader segment (exon 2); 100-187 nt third leader segment (exon 3); 188-301 nt contains the nt sequence immediately following the third leader in the genome with an unknown function; and 322-327 nt BgIII site.

The pCLF plasmid has been deposited with the ATCC as described in Example 4. The complete nucleotide sequence of pCLF is listed in SEQ ID NO: 8 where the nucleotide position 1 corresponds to approximately the middle of the pcDNA3 parent vector sequence. The 5′ and 3′ ends of the Ad5 fiber gene are located at respective nucleotide positions 1237-1239 with ATG and 2980-2982 with TAA.

C. Generation of an Adenovirus Packaging Cell Line Carrying Plasmids Encoding Functional E4 and Fiber Proteins

The 293 cell line was selected for preparing the first adenovirus packaging line as it already contains the E1 gene as prepared by Graham et al., J. Gen. Virol., 36:59-74 (1977) and as further characterized by Spector, Virol., 130:533-538 (1983). Before electroporation, 293 cells were grown in RPMI medium+10% fetal calf serum. Four×106 cells were electroporated with 20 μg each of pE4/Hygro DNA and pCLF DNA using a BioRad GenePulser and settings of 300 V, 25 μF. DNA for electroporation was prepared using the Qiagen system according to the manufacturer's instructions (Bio-Rad, Richmond, Calif.).

Following electroporation, cells were split into fresh complete DMEM+10% fetal calf serum containing 200 μg/ml Hygromycin B (Sigma, St. Louis, Mo.).

From expanded colonies, genomic DNA was isolated using the “MICROTURBOGEN” system (Invitrogen) according to manufacturer's instructions. The presence of integrated E4 DNA was assessed by PCR using 30 the primer pair E4R and ORF6L (5′TGCTTAAGCGGCCGCGAAGGAGA AGTCC3′) (SEQ ID NO: 9), the latter of which is a 5′ forward primer near adenovirus 5 open reading frame 6.

One clone, designated 211, was selected exhibiting altered growth properties relative to that seen in parent cell line 293. The 211 clone contained the product,indicating the presence of inserted DNA corresponding to most, if not all, of the E4 fragment contained in the pE4/Hygro plasmid. The 211 cell line has been deposited with the ATCC as described in Example 4. This line was further evaluated by amplification using the primer pair E4L/E4R described above, and a product corresponding to the full-length E4 insert was detected. Genomic Southern blotting was performed on DNA restricted with EcoRI and BamHI. The E4 fragment was then detected at approximately one copy/genome compared to standards with the EcoRI/BamHI E4 fragment as cloned into pBS/E4 for use as a labeled probe with the Genius system according to manufacturer's instructions (Boehringer Mannheim, Indianapolis, Ind.). In DNA from the 211 cell line, the labeled internal fragment pE4/Hygro hybridized with the isolated E4 sequences. In addition, the probe hybridized to a larger fragment which may be the result of a second insertion event.

Although the 211 cell line was not selected by neomycin resistance, thus indicating the absence of fiber gene, to confirm the lack of fiber gene, the 211 cell line was analyzed for expression of fiber protein by indirect immunofluorescence with an anti-fiber polyclonal antibody and a FITC-labeled anti-rabbit IgG (KPL) as secondary. No immunoreactivity was detected. Therefore, to generate 211 clones containing recombinant fiber genes, the 211 clone was expanded by growing in RPMI medium and subjected to additional electroporation with the fiber-encoding pCLF plasmid as described above.

Following electroporation, cells were plated in DMEM+10% fetal calf serum and colonies were selected with 200 μg/ml G418 (Gibco, Gaithersburg, Md.). Positive cell lines remained hygromycin resistant. These candidate sublines of 211 were then screened for fiber protein expression by indirect immunofluorescence as described above. The three sublines screened, 211A, 211B and 211R, along with a number of other sublines, all exhibited nuclear staining qualitatively comparable to the positive control of 293 cells infected with AdRSVβgal (1 pfu/cell) and stained 24 hours post-infection.

Lines positive for nuclear staining in this assay were then subjected to Western blot analysis under denaturing conditions using the same antibody. Several lines in which the antibody detected a protein of the predicted molecular weight (62 kd for the Ad5 fiber protein) were selected for further study including 211A, 211B and 211R. The 211A cell line has been deposited with ATCC as described in Example 4.

Immunoprecipitation analysis using soluble nuclear extracts from these three cell lines and a seminative electrophoresis system demonstrated that the fiber protein expressed is in the functional trimeric form characteristic of the native fiber protein. The predicted molecular weight of a trimerized fiber is 186 kd. Under denaturing conditions, the trimeric form was destroyed resulting in detectable fiber monomers. Those clones containing endogenous E1, newly expressed recombinant E4 and fiber proteins were selected for use in complementing adenovirus gene delivery vectors having the corresponding adenoviral genes deleted as described in Example 2.

D. Preparation of an E1-Expressing Plasmid for Complementation of E1-Gene-Deleted Adenoviruses

In order to prepare adenoviral packaging cell lines other than those based on the E1-gene containing 293 cell line as described in Example 1C above, plasmid vectors containing E1 alone or in various combinations with E4 and fiber genes are constructed as described below.

The region of the adenovirus genome containing the E1a and E1b gene is amplified from viral genomic DNA by PCR as previously described. The primers used are E1L, the 5′ or forward primer, and E1R, the 3′ or backward primer, having the respective nucleotide sequences 5′CCG AGCTAGC GACTGAAAATGAG3′ (SEQ ID NO: 10) and 5′CCTCTCGAG AGACAGC AAGACAC3′ (SEQ ID NO: 11). The E1L and E1R primers include the respective restriction sites NheI and XhoI as indicated by the underlines. The sites are used to clone the amplified E1 gene fragment into the NheI/XhoI sites in pMAM commercially available from Clontech (Palo Alto, Calif.) to form the plasmid pDEX/E1 having 11152 bp.

The complete nucleotide sequence of pDEX/E1 is listed in SEQ ID NO: 12 where the nucleotide position 1 corresponds to approximately 1454 nucleotides from the 3′ end of the pMAM backbone vector sequence. The pDEX/E1 plasmid includes nucleotides 552 to 4090 of the adenovirus genome positioned downstream (beginning at nucleotide position 1460 and ending at 4998 in the pDEX/E1 plasmid) of the glucocorticoid-inducible mouse mammary tumor virus (MMTV) promoter of pMAM. The pMAM vector contains the E. coli gpt gene that allows stable transfectants to be isolated using hypoxanthine/amino-pterin/thymidine (HAT) selection. The pMAM backbone occupies nucleotide positions 1-1454 and 5005-11152 of SEQ ID NO: 12.

E. Generation of an Adenovirus Packaging Cell Line Carrying Plasmids Encoding Functional E1, and Fiber Proteins

To create separate adenovirus packaging cell lines equivalent to that of the 211 sublines, 211A, 211B and 211R, as described in Example 1C, alternative cell lines lacking adenoviral genomes are selected for transfection with the plasmid constructs as described below. Acceptable host cells include A549, Hela, Vero and the like cell lines as described in Example 1. The selected cell line is transfected with the separate plasmids, pDEX/E1 and pCLF, respectively for expressing E1, and fiber complementary proteins. Following transfection procedures as previously described, clones containing stable insertions of the two plasmids are isolated by selection with neomycin and HAT. Integration of full-length copy of the E1 gene is assessed by PCR amplification from genomic DNA using the primer set E1L/E1R, as described above. Functional insertion of the fiber gene is assayed by staining with the anti-fiber antibody as previously described.

The resultant stably integrated cell line is then used as a packaging cell system to complement adenoviral gene delivery vectors having the corresponding adenoviral gene deletions as described in Example 2.

F. Preparation of a Plasmid Containing Two or More Adenoviral Genes for Complementing Gene-Deleted Adenoviruses

The methods described in the preceding Examples rely on the use of two plasmids, pE4/Hygro and pCLF, or, pCLF and pDEX/E1 for generating adenoviral cell packaging systems. In alternative embodiments, complementing plasmids containing two or more adenoviral genes for expressing of encoded proteins in various combinations are also prepared as described below. The resultant plasmids are then used in various cell systems with delivery plasmids having the corresponding adenoviral gene deletions. The selection of packaging cell, content of the delivery plasmids and content of the complementing plasmids for use in generating recombinant adenovirus viral vectors thus depends on whether other adenoviral genes are deleted along with the adenoviral fiber gene, and, if so, which ones.

1. Preparation of a Complementing Plasmid Containing Fiber and E1 Adenoviral Genes

A DNA fragment containing sequences for the CMV promoter, adenovirus tripartite leader, fiber gene and bovine growth hormone terminator is amplified from pCLF prepared in Example 1B using the forward primer 5′GACGGATCGGGAGATCTCC3′ (SEQ ID NO: 13), that anneals to the nucleotides 1-19 of the pCDNA3 vector backbone in pCLF, and the backward primer 5′CCGCCTCAGAAGCCATAGAGCC3′ (SEQ ID NO: 14) that anneals to nucleotides 1278-1257 of the pCDNA3 vector backbone. The fragment is amplified as previously described and then cloned into the pDEX/E1 plasmid, prepared in Example 1D. For cloning in the DNA fragment, the pDEX/E1 vector is first digested with Ndel, that cuts at a unique site in the pMAM vector backbone in pDEX/E1, then the ends are repaired by treatment with bacteriophage T4 polymerase and dNTPs.

The resulting plasmid containing E1 and fiber genes, designated pE1/Fiber, provides dexamethasone-inducible E1 function as described for DEX/E1 and expression of Ad5 fiber protein as described above.

The complete nucleotide sequence of pE1/Fiber is listed in SEQ ID NO: 15 where the nucleotide position 1 corresponds to approximately 1459 nucleotides from the 3′ end of the parent vector pMAM sequence. The 5′ and 3′ ends of the Ad5 E1 gene are located at respective nucleotide positions 1460 and 4998 followed by pMAM backbone and then separated from the Ad5 fiber from pCLF by the filled-in blunt ended Ndel site. The 5′ and 3′ ends of the pCLF fiber gene fragment are located at respective nucleotide positions 10922-14223 containing elements as previously described for pCLF.

The resultant pE1/Fiber plasmid is then used to complement one or more delivery plasmids expressing E1 and fiber.

The pE1/Fiber construct is then used to transfect a selected host cell as described in Example 1E to generate stable chromosomal insertions preformed as previously described followed by selection on HAT medium. The stable cells are then used as packaging cells as described in Example 2.

2. Preparation of a Complementing Plasmid Containing E4 and Fiber Adenoviral Genes

Plasmid pCLF prepared as described in Example 1B is partially digested with BgIII to cut only at the site in the pCDNA3 backbone. The pE4/Hygro plasmid prepared in Example 1A is digested with BamHI to produce a fragment containing E4. The E4 fragment is then inserted into the BamHI site of pCLF to form plasmid pE4/Fiber. The resultant plasmid provides expression of the fiber gene as described for pCLF and E4 function as described for pE4/Hygro.

A schematic plasmid map of pE4/Fiber, having 10610 bp. The complete nucleotide sequence of pE4/Fiber is listed in SEQ ID NO: 16 where the nucleotide position 1 corresponds to approximately 14 bp from the 3′ end of the parent vector pCDNA3 backbone sequence. The 5′ and 3′ ends of the Ad5 E4 gene are located at respective nucleotide positions 21 and 3149 followed by fused BgIII/BamHI sites and pCDNA3 backbone including the CMV promoter again followed by BgIII/BamHI sites. The adenovirus leader sequence begins at nucleotide position 4051 and extends to 4366 followed by fused BamHI/BgIII sites and the 5′ and 3′ ends of the fiber gene located at respective nucleotide positions 4372 and 6124.

Stable chromosonal insertions of pE4/Fiber in host cells are obtained as described above.

### EXAMPLE 2

Preparation of Adenoviral Gene Delivery Vectors Using Adenoviral Packaging Cell Lines

Adenoviral delivery vectors are prepared to separately lack the combinations of E1/fiber and E4/fiber. Such vectors are more replication-defective than those previously in use due to the absence of multiple viral genes. A preferred adenoviral delivery vector is replication competent but only via a non-fiber means is one that only lacks the fiber gene but contains the remaining functional adenoviral regulatory and structural genes. Furthermore, these adenovirus delivery vectors have a higher capacity for insertion of foreign DNA.

A. Preparation of Adenoviral Gene Delivery Vectors Having Specific Gene Deletions and Methods of Use

To construct the E1/fiber deleted viral vector containing the LacZ reporter gene construct, two new plasmids were constructed. The plasmid pΔE1Bβgal was constructed as follows. A DNA fragment containing the SV40 regulatory sequences and E. coli β-galactosidase gene was isolated from pSVβgal (Promega) by digesting with Vspl, filling the overhanging ends by treatment with Klenow fragment of DNA polymerase I in the presence of dNTP's and digesting with Bam H1. The resulting fragment was cloned into the EcoRV and BamHI sites in the polylinker of pΔ E1sp1B (Microbix Biosystems, Hamilton, Ontario) to form pΔ E1B βgal that therefore contained the left end of the adenovirus genome with the EIa region replaced by the LacZ cassette (nucleotides 6690 to 4151) of pSVβ gal. Plasmid DNA may be prepared by the alkaline lysis method as described by Birnboim and Doly, Nuc. Acids Res., 7:1513-1523 (1978) or by the Quiagen method according to the manufacturer's instruction, from transformed cells used to expand the plasmid DNA was then purified by CsCl-ethidium bromide density gradient centrifugation. Alternatively, plasmid DNAs may be purified from E. coli by standard methods known in the art (e.g. see Sambrook et al.)

The second plasmid (pDV44), prepared as described herein, is derived from pBHG10, a vector prepared as described by Bett et al., Proc. Natl. Acad. Sci., USA, 91:8802-8806 (1994) (see, also International PCT application No. WO 95/00655) using methods well known to one of skill in the art. This vector is also commercially available from Microbix and and contains an Ad5 genome with the packaging signals at the left end deleted and the E3 region (nucleotides 28133:30818) replaced by a linker with a unique site for the restriction enzyme PacI. An 11.9 kb BamHI fragment, which contains the right end of the adenovirus genome, is isolated from pBHG10 and cloned into the BamHI site of pBS/SK(+) to create plasmid p11.3 having approximately 14,658 bp. The p11.3 plasmid was then digested with PacI and SalI to remove the fiber, E4, and inverted terminal repeat (ITR) sequences.

This fragment was replaced with a 3,4 kb fragment containing the ITR segments and the E4 gene which was generated by PCR amplification from pBHG10 using the following oligonucleotide sequences: 5′ TGTACACCG GATCCGGCGCACACC3′ SEQ ID NO: 17; and 5′CACAACGAGCTC AATTAATTAATTGCCACATCCTC3′ SEQ ID NO: 18. These primers incorporated sites for PacI and BamHI. Cloning this fragment into the PacI and blunt ended SalI sites of the p11.3 backbone resulted in a substitution of the fused ITRs, E4 region and fiber gene present in pBHG10, by the ITRs and E4 region alone. The resulting p11.3 plasmid containing the ITR and E4 regions, designated plasmid pDV43a, was then digested with BamHI. This BamHI fragment was then used to replace a BamHI fragment in pBHG10 thereby creating pDV44 in a pBHG10 backbone.

In an alternative approach to preparing pDV44 with an additional subcloning step to facilitate the incorporation of restriction cloning sites, the following cloning procedure was performed. pDV44 as above was constructed by removing the fiber gene and some of the residual E3 sequences from pBHG10 (Microbix Biosystems). As above, to simplify manipulations, the 11.9 kb BamHI fragment including the rightmost part of the Ad5 genome was removed from pBHG10 and inserted into pBS/SK. The resulting plasmid was termed p11.3. The 3.4 kb DNA fragment corresponding to the E4 region and both ITRs of adenovirus type 5 was amplified as described above from pBHG10 using the oligonucleotides listed above and subcloned into the vector pCR2.1 (Invitrogen) to create pDV42. This step is the additional cloning step to facilitate the incorporation of a SalI restriction site. pDV42 was then digested with PacI, which cuts at a unique site (bold type) in one of the PCR primers, and with SalI, which cuts at a unique site in the pCR2.1 polylinker. This fragment was used to replace the corresponding PacI/XhoI fragment of p11.3 (the pBS polylinker adjacent to the Ad DNA fragment contains a unique XhoI site), creating pDV43.

A plasmid designated pDV44 was constructed by replacing the 11.9 kb BamHI fragment of pBHG10 by the analogous BamHI fragment of pDV43. As generated in the first procedure, pDV44 therefore differs from pBHG10 by the deletion of Ad5 nucleotides 30819:32743 (residual E3 sequences and all but the 3′-most 41 nucleotides of the fiber open reading frame).

Thus, to summarize, the cloning procedures described above result in the production of a fiber-deleted Ad5 genomic plasmid (pDV44) that was constructed by removing the fiber gene and some of the residual E3 sequences from pBHG10. pDV44 contains a wild-type E4 region, but only the last 41 nucleotides of the fiber ORF (this sequence was retained to avoid affecting expression of the adjacent E4 transcription unit). Plasmids pBHG10 and pDV44 contain unpackageable Ad5 genomes, and must be rescued by cotransfection and subsequent homologous recombination with DNA carrying functional packaging signals. In order to generate vectors marked with a reporter gene, either pDV44 or pBHG10 was cotransfected with pΔE1Bβgal, which contains the left end of the Ad5 genome with an SV40-driven β-galactosidase reporter gene inserted in place of the E1 region.

In general, and as described below, the method for virus production by recombination of plasmids followed by complementation in cell culture involves the isolation of recombinant viruses by cotransfection of any one of the adenovirus packaging cell systems prepared in Example 1, namely 211A, 211B, 211R, A549, Vero cells, and the like, with plasmids carrying sequences corresponding to viral gene delivery vectors.

A selected cell line is plated in dishes and cotransfected with pDV44 and pΔE1Bβ gal using the calcium phosphate method as described by Bett et al., Proc. Natl. Acad. Sci., USA, 91:8802-8806 (1994). Recombination between the overlapping adenovirus sequences in the two plasmids leads to the creation of a full-length viral chromosome where pDV44 and pΔE1Bβ gal recombine to form a recombinant adenovirus vector having multiple deletions. The deletion of E1 and of the fiber gene from the viral chromosome is compensated for by the sequences integrated into the packaging cell genome, and infectious virus particles are produced. The plaques thus generated are isolated and stocks of the recombinant virus are produced by standard methods.

Because of the fiber deletion, a pDV44-derived virus is replication-defective, cells in which it is grown must complement this defect. The 211B cell line (a derivative of 293 cells which expresses the wild-type (wt) AD5 fiber and is equivalent to 211A on deposit with ATCC as described in Example 4) was used for rescue and propagation of the virus described here. pDV44 and pΔE1βgal were cotransfected into 211B cells, and the monolayers were observed for evidence of cytopathic effect (CPE). Briefly, for virus construction, cells were transfected with the indicated plasmids using the Gibco Calcium Phosphate Transfection system according to the manufacturer's instructions and observed daily for evidence of CPE.

One of a total of 58 transfected dishes showed evidence of spreading cell death at day 15. A crude freeze-thaw lysate was prepared from these cells and the resulting virus (termed Ad5.βgal.ΔF) was plaque purified twice and then expanded. To prepare purified viral preparations, cells were infected with the indicated Ad and observed for completion of CPE. Briefly, at day zero, 211B cells were plated in DMEM plus 10% fetal calf serum at approximately 1×10⁷ cells/150 cm² flask or equivalent density. At day one, the medium was replaced with one half the original volume of fresh DMEM containing the indicated Ad, in this case Ad5.βgal.ΔF, at approximately 100 particles/cell. At day two, an equal volume of medium was added to each flask and the cells were observed for CPE. Two to five days after infection, cells were collected and virus isolated by lysis via four rapid freeze-thaw cycles. Virus was then purified by centrifugation on preformed 15-40% CsCl gradients (111,000×g for three hours at 4° C.). The bands were harvested, dialyzed into storage buffer (10 mM Tris-pH 8.1, 0.9% NaCl, and 10% glycerol), aliquoted and stored at −70° C. Purified Ad5.βgal.ΔF virus particles containing human adenovirus Ad5.βgal.ΔFgenome (described further below) have been deposited with the ATCC on Jan. 15, 1999 as further described in Example 4.

For viral titering, as necessary in the below Examples, Ad preparations were titered by plaque assay on 211B cells. Cells were plated on polylysine-coated 6 well plates at 1.5×10⁶ cells/well. Duplicate dilutions of virus stock were added to the plates in 1 ml/well of complete DMEM. After a five hour incubation at 37° C., virus was removed and the wells overlaid with 2 ml of 0.6% low-melting agarose in Medium 199 (Gibco). An additional 1 ml of overlay was added at five day intervals.

As a control, the first-generation virus Ad5.β gal.wt, which is identical to Ad5.βgal.ΔF except for the fiber deletion, was constructed by cotransfection of pBHG10 and pΔE1Bβgal. In contrast to the low efficiency of recovery of the fiberless genome (1/58 dishes), all of 9 dishes cotransfected with pΔE1Bβgal and pBHG10 produced virus.

In another embodiment, a delivery plasmid is prepared that does not require the above-described recombination events to prepare a viral vector having a fiber gene deletion. In one embodiment, a single delivery plasmid containing all the adenoviral genome necessary for packaging but lacking the fiber gene is prepared from plasmid pFG140 containing full-length Ad5 that is commercially available from Microbix. The resultant delivery plasmid referred to as pFG140-f is then used with pCLF stably integrated cells as described above to prepare a viral vector lacking fiber. For genetic therapy, the fiber gene can be replaced with a therapeutic gene of interest for preparing a therapeutic delivery adenoviral vector. Methods for producing a fiberless vector with a complete TPL are described in Example 3.

Vectors for the delivery of any desired gene and preferably a therapeutic gene are prepared by cloning the gene of interest into the multiple cloning sites in the polylinker of commercially available pΔE1sp1B (Microbix Biosystems), in an analogous manner as performed for preparing pE1Bβ gal as described above. The same cotransfection and recombination procedure is then followed as described herein to obtain viral gene delivery vectors as further discussed in later Examples.

1. Characterization of the Ad5.βgal.ΔF Genome

To confirm that the vector genomes had the proper structures and that the fiber gene was absent from the Ad5.βgal.ΔF chromosome, the DNA isolated from viral particles was analyzed. Briefly, purified viral DNA was obtained by adding 10 μl of 10 mg/ml proteinase K, 40 μl of 0.5 M EDTA and 50 μl of 10% SDS to 800 μl of adenovirus-containing culture supernatant. The suspension was then incubated at 55° C. for 60 minutes. The solution was then extracted once with 400 μl of a 24:1 mixture of chloroform:isoamyl alchohol. The aqueous phase was then removed and precipitated with sodium acetate/ethanol. The pellet was washed once with 70% ethanol and lightly dried. The pellet was then suspended in 40 μl of 10 mM Tris-HCl, pH 8.0, 1 mM EDTA. Genomic DNA from Ad5.βgal.wt and Ad5.βgal.ΔF produced the expected restriction patterns following digestion with either EcoRI or with Ndel. Southern blotting, performed with standard methods, with labeled fiber DNA as a probe demonstrated the presence of fiber sequence in Ad5.βgal.wt but not in Ad5.βgal.ΔF DNA. As a positive control, the blot was stripped and reprobed with labeled E4 sequence. Fiber and E4 sequences were detected by using labeled inserts from pCLF and pE4/Hygro, respectively. E4 signal was readily detectable in both genomes at equal intensities. The complete nucleotide sequence of Ad5.βgal.ΔF is presented in SEQ ID NO: 23 and is contained in the virus particle on deposit with ATCC.

2. Characterization of the Fiberless Adenovirus Ad5.βgal.ΔF

To verify that Ad5.βgal.ΔF was fiber-defective, 293 cells (which are permissive for growth of E1-deleted Ad vectors but do not express fiber) were infected with Ad5.βgal.ΔF or with Ad5.βgal.wt. Twenty-four hours post infection, the cells were stained with polyclonal antibodies directed either against fiber or against the penton base protein. Cells infected with either virus were stained by the anti-penton base antibody, while only cells infected with the Ad5.βgal.wt control virus reacted with the anti-fiber antibody. This confirms that the fiber-deleted Ad mutant does not direct the synthesis of fiber protein.

3. Growth of the Fiber-Deleted Ad5.βgal.ΔF Vector in Complementing Cells

Ad5.βgal.ΔF was found to readily be propagated in 211B cells. As assayed by protein concentration, CsCl-purified stocks of either Ad5.βgal.ΔF or Ad5.βgal.wt contained similar numbers of viral particles. The particles appeared to band normally on CsCl gradients. Infectivity of the Ad5.βgal.ΔF particles was lower than the Ad5.βgal.wt control, as indicated by an increased particle/PFU ratio. Ad5.βgal.ΔF was also found to plaque more slowly than the control virus. When plated on 211B cells, Ad5.βgal.wt plaques appeared within 5-7 days, while plaques of Ad5.βgal.ΔF continued to appear until as much as 15-18 days post infection. Despite their slower formation, the morphology of Ad5.βgal.ΔF plaques was essentially normal.

4. Production of Fiberless Ad5.βgal.ΔF Particles

As Ad5.βgal.ΔF represents a true fiber null mutation and its stocks are free of helper virus, the fiber mutant phenotype was readily investigated. A single round of growth in cells (such as 293) which do not produce fiber generating a homogeneous preparation of fiberless Ad allowed for the determination of whether such particles would be stable and/or infectious. Either Ad5.βgal.wt or Ad5.βgal.ΔF was grown in 293 or 211B cells, and the resulting particles purified on CsCl gradients as previously described. Ad5.βgal.ΔF particles were readily produced in 293 cells at approximately the same level as the control virus and behaved similarly on the gradients, indicating that there was not a gross defect in morphogenesis of fiberless capsids.

Particles of either virus contained similar amounts of penton base regardless of the cell type in which they were grown. This demonstrated that fiber is not required for assembly of the penton base complex into virions. The Ad5.βgal.ΔF particles produced in 293 cells did not contain fiber protein. 211B-grown Ad5.βgal.ΔF also contained less fiber than the Ad5.βgal.wt control virus. The infectivities of the different viral preparations on epithelial cells correlated with the amount of fiber protein present. The fiberless Ad particles were several thousand-fold less infectious than the first-generation vector control on a per-particle basis, while infectivity of 211B-grown Ad5.βgal.ΔF was only 50-100 fold less than that of Ad5.βgal.wt. These studies confirmed fiber's crucial role in infection of epithelial cells via CAR binding.

5. Composition and Structure of the Fiberless Ad5.βgal.ΔF Particles

The proteins contained in particles of 293-grown Ad5.βgal.ΔF were compared to those in Ad5.βgal.wt, to determine whether proteolysis or particle assembly was defective in this fiber null mutant. The overall pattern of proteins in the fiberless particles was observed to be quite similar to that of a first-generation vector, with the exception of reduced intensity of the composite band resulting from proteins IIIa and IV (fiber). The fiberless particles also had a reduced level of protein VII. Although substantial amounts of uncleaved precursors to proteins VI, VII, and VIII were not seen, it is possible that the low-molecular weight bands migrating ahead of protein VII represent either aberrantly cleaved viral proteins or their breakdown products.

Cryo-electron microscopy was used to more closely examine the structure of the 293 grown Ad5.βgal.ΔF and of Ad5βgal.wt. The fiber, having an extended stalk with a knob at the end, was faintly visible in favorable orientations of wild-type Ad5 particles, but not in images of the fiberless particles. Filamentous material likely corresponding to free viral DNA was seen in micrographs of fiberless particles. This material was also present in micrographs of the first-generation control virus, albeit at much lower levels.

Three-dimensional image reconstructions of fiberless and wild-type particles at ˜20 Å resolution showed similar sizes and overall features, with the exception that fiberless particles lacked density corresponding to the fiber protein. The densities corresponding to other capsid proteins, including penton base and proteins IIIa, VI, and IX, were comparable in the two structures. This confirms that absence of fiber does not prevent assembly of these components into virions. The fiber was truncated in the wild-type structure as only the lower portion of its flexible shaft follows icosahedral symmetry. The RGD protrusions on the fiberless penton base were angled slightly inward relative to those of the wild-type structure. Another difference between the two penton base proteins was that there is a ˜30 Å diameter depression in the fiberless penton base around the five-fold axis where the fiber would normally sit. The Ad5 reconstructions confirm that capsid assembly, including addition of penton base to the vertices, is able to proceed in the complete absence of fiber.

6. Integrin-Dependent Infectivity of Fiberless Ad5.βgal.ΔF Particles

While attachment via the viral fiber protein is a critical step in the infection of epithelial cells, an alternative pathway for infection of certain hematopoietic cells has been described. In this case, penton base mediates binding to the cells (via β2 integrins) and internalization (through interaction with αv integrins). Particles lacking fiber might therefore be expected to be competent for infection of these cells, even though on a per-particle basis they are several thousand-fold less infectious than normal Ad vectors on epithelial cells.

To investigate this, THP-1 monocytic cells were infected with Ad5.βgal.wt or with Ad5.βgal.ΔF grown in the absence of fiber. Infection of THP-1 cells was assayed by infecting 2×10⁵ cells at the indicated m.o.i. in 0.5 ml of complete RPMI. Forty-eight hours post-infection, the cells were fixed with glutaraldehyde and stained with X-gal, and the percentage of stained cells was determined by light microscopy. The results of the infection assay showed that the fiberless particles were only a few-fold less infectious than first-generation Ad on THP-1 cells. Large differences were seen in plaquing efficiency on epithelial (211B) cells. Infection of THP-1 cells by either Ad5.βgal.ΔF or Ad5.βgal.wt was not blocked by an excess of soluble recombinant fiber protein, but could be inhibited by the addition of recombinant penton base). These results indicate that the fiberless Ad particles use a fiber-independent pathway to infect these cells. Furthermore, the lack of fiber protein did not prevent Ad5.βgalΔF from internalizing into the cells and delivering its genome to the nucleus, demonstrating that fiberless particles are properly assembled and are capable of uncoating.

The foregoing results with the recombinant viruses thus produced indicates that they can be used as gene delivery tools in cultured cells and in vivo as described more fully in the Examples. For example, for studies of the effectiveness and relative immunogenicity of multiply-deleted vectors, virus particles are produced by growth in the packaging lines described in Example 1 and are purified by CsCl gradient centrifugation. Following titering, virus particles are administered to mice via systemic or local injection or by aerosol delivery to lung. The LacZ reporter gene allows the number and type of cells which are successfully transduced to be evaluated. The duration of transgene expression is evaluated in order to determine the long-term effectiveness of treatment with multiply-deleted recombinant adenoviruses relative to the standard technologies which have been used in clinical trials to date. The immune response to the improved vectors described here is determined by assessing parameters such as inflammation, production of cytotoxic T lymphocytes directed against the vector, and the nature and magnitude of the antibody response directed against viral proteins.

Versions of the vectors which contain therapeutic genes such as CFTR for treatment of cystic fibrosis or tumor suppressor genes for cancer treatment are evaluated in the animal system for safety and efficiency of gene transfer and expression. Following this evaluation, they are used as experimental therapeutic agents in human clinical trials.

B. Retargeting of Adenoviral Gene Delivery Vectors by Producing Viral Particles Containing Different or Altered Fiber Proteins

As the specificity of adenovirus binding to target cells is largely determined by the fiber protein, viral particles that incorporate modified fiber proteins or fiber proteins from different adenoviral serotypes (pseudotyped vectors) have different specificities. Thus, the methods of expression of the native Ad5 fiber protein in adenovirus packaging cells as described above is also applicable to production of different fiber proteins.

Chimeric fiber proteins can be produced according to known methods (see, e.g., Stevenson et al. (1995) J. Virol., 69:2850-2857). Determinants for fiber receptor binding activity are located in the head domain of the fiber and an isolated head domain is capable of trimerization and binding to cellular receptors. The head domains of adenovirus type 3 (Ad3) and Ad5 were exchanged in order to produce chimeric fiber proteins. Similar constructs for encoding chimeric fiber proteins for use in the methods herein are contemplated. Thus, instead of the using the intact Ad5 fiber-encoding construct prepared in above and in U.S. application Ser. No. 09/482,682) as a complementing viral vector in adenoviral packaging cells, the constructs described herein are used to transfect cells along with E4 and/or E1-encoding constructs.

Briefly, full-length Ad5 and Ad3 fiber genes were amplified from purified adenovirus genomic DNA as a template. The Ad5 and Ad3 nucleotide sequences are available with the respective GenBank Accession Numbers M18369 and M12411. Oligonucleotide primers are designed to amplify the entire coding sequence of the full-length fiber genes, starting from the start codon, ATG, and ending with the termination codon TAA. For cloning purposes, the 5′ and 3′ primers contain the respective restriction sites BamHI and NotI for cloning into pcDNA plasmid as described in Example 1A. PCR is performed as described above.

The resulting products are then used to construct chimeric fiber constructs by PCR gene overlap extension (Horton et al. (1990) BioTechniques, 8:525-535). The Ad5 fiber tail and shaft regions (5TS; the nucleotide region encoding amino acid residue positions 1 to 403) are connected to the Ad3 fiber head region (3H; the nucleotide region encoding amino acid residue positions 136 to 319) to form the 5TS3H fiber chimera. Conversely, the Ad3 fiber tail and shaft regions (3TS; the nucleotide region encoding amino acid residues positions 1 to 135) are connected to the Ad5 fiber head region (5H; the nucleotide region encoding the amino acid residue positions 404 to 581) to form the 3TS5H fiber chimera. The fusions are made at the conserved TLWT (SEQ ID NO: 19) sequence at the fiber shaft-head junction.

The resultant chimeric fiber PCR products are then digested with BamHI and NotI for separate directional ligation into a similarly digested pcDNA 3.1. The TPL sequence is then subcloned into the BamHI as described in Example 1A for preparing an expression vector for subsequent transfection into 211 cells as described above or into the alternative packaging cell systems as previously described. The resultant chimeric fiber construct-containing adenoviral packaging cell lines are then used to complement adenoviral delivery vectors as previously described. Other fiber chimeric constructs are obtained with the various adenovirus serotypes using a similar approach.

In an alternative embodiment, the use of modified proteins including with modified epitopes (see, e.g., Michael et al. (1995) Gene Therapy, 2:660-668 and International PCT application Publication No. WO 95/26412, which describe the construction of a cell-type specific therapeutic viral vector having a new binding specificity incorporated into the virus concurrent with the destruction of the endogenous viral binding specificity). In particular, the authors described the production of an adenoviral vector encoding a gastrin releasing peptide (GRP) at the 3′ end of the coding sequence of the Ad5 fiber gene. The resulting fiber-GRP fusion protein was expressed and shown to assemble functional fiber trimers that were correctly transported to the nucleus of HeLa cells following synthesis.

Similar constructs are contemplated for use in the complementing adenoviral packaging cell systems for generating new adenoviral gene delivery vectors that are targetable, replication-deficient and less immunogenic. Heterologous ligands contemplated for use herein to redirect fiber specificity range from as few as 10 amino acids in size to large globular structures, some of which necessitate the addition of a spacer region so as to reduce or preclude steric hindrance of the heterologous ligand with the fiber or prevent trimerization of the fiber protein. The ligands are inserted at the end or within the linker region. Preferred ligands include those that target specific cell receptors or those that are used for coupling to other moieties such as biotin and avidin.

A preferred spacer includes a short 12 amino acid peptide linker composed of a series of serines and alanine flanked by a proline residue at each end using routine procedures known to those of skill in the art. The skilled artisan will be with the preparation of linkers to accomplish sufficient protein presentation and to alter the binding specificity of the fiber protein without compromising the cellular events that follow viral internalization. Moreover, within the context of this disclosure, preparation of modified fibers having ligands positioned internally within the fiber protein and at the carboxy terminus as described below are contemplated for use with the methods described herein.

The preparation of a fiber having a heterologous binding ligand is prepared essentially as described in the above-cited paper. Briefly, for the ligand of choice, site-directed mutagenesis is used to insert the coding sequence for a linker into the 3′ end of the Ad5 fiber construct in pCLF as prepared in Example 1.

The 3′ or antisense or mutagenic oligonucleotide encodes a preferred linker sequence of ProSerAlaSerAlaSerAlaSerAlaProGlySer (SEQ ID NO: 20) followed by a unique restriction site and two stop codons, respectively, to allow the insertion of a coding sequence for a selected heterologous ligand and to ensure proper translation termination. Flanking this linker sequence, the mutagenic oligonucloetide contains sequences that overlap with the vector sequence and allow its incorporation into the construct. Following mutagenesis of the pCLF sequence adding the linker and stop codon sequences, a nucleotide sequence encoding a preselected ligand is obtained, linkers corresponding to the unique restriction site in the modified construct are attached and then the sequence is cloned into linearized corresponding restriction site. The resultant fiber-ligand construct is then used to transfect 211 or the alternative cell packaging systems previously described to produce complementing viral vector packaging systems.

In a further embodiment, intact fiber genes from different Ad serotypes are expressed by 211 cells or an alternative packaging system as previously described. A gene encoding the fiber protein of interest is first cloned to create a plasmid analogous to pCLF, and stable cell lines producing the fiber protein are generated as described above for Ad5 fiber. The adenovirus vector described which lacks the fiber gene is then propagated in the cell line producing the fiber protein relevant for the purpose at hand. As the only fiber gene present is the one in the packaging cells, the adenoviruses produced contain only the fiber protein of interest and therefore have the binding specificity conferred by the complementing protein. Such viral particles are used in studies such as those described above to determine their properties in experimental animal systems.

### EXAMPLE 3

Tripartite leader sequences (TPLs) that are useful in enhancing the expression of complementing adenoviral proteins, particularly fiber protein, for use in preparing an adenoviral gene delivery vector are provided. The complete Ad5 TPL was constructed by assembling PCR fragments. First, the third TPL exon (exon 3) (nt 9644-9731 of the Ad5 genome) was amplified from Ad5 genomic DNA using the synthetic oligonucleotide primers

| | |
|---|---|
| 5′CTCAACAATTGTGGATCCGTACTCC3′ | (SEQ ID NO. 24) and |
| 5′GTGCTCAGCAGATCTTGCGACTGTG3′ | (SEQ ID No. 25). |

The resulting product was cloned to the BamHI and BgIII sites of pΔE1Sp1a (Microbix Biosystems) using sites in the primers (shown in bold) to create plasmid pDV52. A fragment corresponding to the first TPL exon (exon 1), the natural first intron (intron 1), and the second TPL exon (exon 2) (Ad5 nt 6049-7182) was then amplified using primers

| | |
|---|---|
| 5′GGCGCGTTCGGGATCCACTCTCTTCC3′ | (SEQ ID No. 26) |
| | and |
| 5′CTACATGCTAGGCAGATCTCGTTCGGAG3′ | (SEQ ID No. 27), |
| | and |

Cloned Into the BamHI Site of pDV52 (again using sites in the primers) to create pDV55.

This plasmid contains a 1.2 kb BamHI/BgIII fragment containing the first TPL exon, the natural first intron, and the fused second and third TPL exons. The nucleotide sequence of the complete TPL containing the noted 5′ and 3′ restriction sites is shown in SEQ ID No 28 with the following nucleotide regions identified: 1-6 nt BamHI site; 7-47 nt first leader segment (exon 1); 48-1068 nt natural first intron (intron 1); 1069-1140 nt second leader segment (exon 2); 1141-1146 nt fused BamHI and BgIII sites; 1147-1234 nt third leader segment (exon 3); and 1235-1240 nt BgIII site.

### EXAMPLE 4

Deposit of Materials

The following cell lines and plasmids were deposited on Sep. 25, 1996, with the American Type Culture Collection, 10801 University Blvd, Manassas, Va., USA (ATCC) under the provisions of the Budapest Treaty on the International Recognition of the Deposit of Microorganisms for the Purpose of Patent Procedure and the Regulations thereunder (Budapest Treaty): Plasmid pE4/Hygro (accession number 97739), Plasmid pCLF (accession number 97737), 211 Cell Line (accession number CRL-12193) and 211A Cell Line (accession number CRL-12194)

The following virus, Ad5.βgal.ΔF, was deposited on Jan. 15, 1999, with the ATCC as listed above and provided with accession number VR2636.

Additionally, plasmids pDV60, pDV67, pDV69, pDV80 and pDV90 were deposited at the ATCC on Jan. 5, 2000 and provided with accession numbers PTA-1144, PTA-1145, PTA-1146, PTA-1147 and PTA-1148 respectively.

### EXAMPLE 5

Preparation and Use of Adenoviral Packaging Cell Lines Containing Plasmids Containing Alternative TPLs

Plasmids containing tripartite leaders (TPLs) have been constructed. The resulting plasmids that contain different selectable markers, such as neomycin and zeocin, were then used to prepare fiber-complementing stable cell lines for use as for preparing adenoviral vectors.

A. pDV60

Plasmid pDV60 was constructed by inserting this TPL cassette of SEQ ID No. 28 into the BamHI site upstream of the Ad5 fiber gene in pcDNA3/Fiber, a neomycin selectable plasmid (see, e.g., U.S. application Ser. No. 09/482,682 (also filed as International PCT application No. PCT/US00/00265 on Jan. 14, 2000); see also Von Seggern et al. (1998) J. Gen Virol., 79: 1461-1468). The nucleotide sequence of pDV60 is listed in SEQ ID NO: 29. Plasmid pDV60 has been deposited in the ATCC under accession number PTA-1144.

B. pDV61

To construct pDV61, an Asp718/NotI fragment containing the CMV promoter, partial Ad5 TPL, wildtype Ad5 fiber gene, and bovine growth hormone terminator was transferred from pCLF (ATCC accession number 97737; and described in copending U.S. application Ser. No. 09/482,682 (also filed as International PCT application No. PCT/US00/00265 on Jan. 14, 2000);), to a zeocin selectable cloning vector referred to as pCDNA3.1/Zeo (+) (commerically available from Invitrogen and for which the sequence is known).

C. pDV67

In an analogous process, pDV67 containing complete TPL was constructed by transferring an Asp 718/XbaI fragment from pDV60 into pcDNA3.1/Zeo(+) backbone. The nucleotide sequence of pDV67 is set forth in SEQ ID No. 30. Plasmid pDV67 is available from the ATCC under accession number PTA-1145.

D. pDV69

To prepare pDV69 containing a modified fiber protein, the chimeric Ad3/Ad5 fiber gene was amplified from pGEM5TS3H (Stevenson et al. (1995) J. Virol., 69: 2850-2857) using the primers

| | |
|---|---|
| 5′ATGGGATCAAGATGAAGCGCGCAAGACCG3′ | (SEQ ID NO. 31) |
| | and |
| 5′CACTATAGCGGCCGCATTCTCAGTCATCTT3′ | (SEQ ID No. 32), |

and cloned to the BamHI and NotI sites of pcDNA3.1/Zeo(+) via new BamHI and NotI sites engineered into the primers to create pDV68. Finally, the complete TPL fragment described above was then added to the unique BamHI site of pDV68 to create pDV69. The nucleotide sequence of pDV69 is listed in SEQ ID No. 33 and has been deposited in the ATCC under accession number PTA-1146.

E. Preparation of Stable Adenovirus Packaging Cell Lines

E1-2a S8 cells are derivatives of the A549 lung carcinoma line (ATCC # CCL 185) with chromosomal insertions of the plasmids pGRE5-2.E1 (also referred to as GRE5-E1-SV40-Hygro construct and listed in SEQ ID No. 34) and pMNeoE2a-3.1 (also referred to as MMTV-E2a-SV40-Neo construct and listed in SEQ ID No. 35), which provide complementation of the adenoviral E1 and E2a functions, respectively. This line and its derivatives were grown in Richter's modified medium (BioWhitaker)+10% FCS. E1-2a S8 cells were electroporated as previously described (Von Seggern et al. (1998) J. Gen Virol., 79: 1461-1468) with pDV61, pDV67, or with pDV69, and stable lines were selected with zeocin (600 μg/ml).

The cell line generated with pDV61 is designated 601. The cell line generated with pDV67 is designated 633 while that generated with pDV69 is designated 644. Candidate clones were evaluated by immunofluorescent staining with a polyclonal antibody raised against the Ad2 fiber. Lines expressing the highest level of fiber protein were further characterized.

For the S8 cell complementing cell lines, to induce E1 expression, 0.3 μM of dexamethasone was added to cell cultures 16-24 hours prior to challenge with virus for optimal growth kinetics. For preparing viral plaques, 5×10⁵ cells/well in 6 well plates are prepared and pre-induced with the same concentration of dexamethasone the day prior to infection with 0.5 μM included at a final concentration in the agar overlay after infection.

F. Development of Cell Lines for Complementation of E1⁻/E2a⁻ Vectors

The Adenovirus 5 genome was digested with Scal enzyme, separated on an agarose gel, and the 6,095 bp fragment containing the left end of the virus genome was isolated. The complete Adenovirus 5 genome is registered as Genbank accession #M73260, incorporated herein by reference, and the virus is available from the American Type Culture Collection, Manassas, Va., U.S.A., under accession number VR-5. The Scal 6,095 bp fragment was digested further with ClaI at bp 917 and BgIII at bp 3,328. The resulting 2,411 bp ClaI to BgIII fragment was purified from an agarose gel and ligated into the superlinker shuttle plasmid pSE280 (Invitrogen, San Diego, Calif.), which was digested with ClaI and BgIII, to form pSE280-E.

Polymerase chain reaction (PCR) was performed to synthesize DNA encoding an XhoI and SalI restriction site contiguous with Adenovirus 5 DNA bp 552 through 924. The primers which were employed were as follows:

| | |
|---|---|
| 5′ end, Ad5 bp 552-585: | |
| 5′-GTCACTCGAGGACTCGGTC-GACTGAAAATGAGACATATTATCTGCCACGGACC-3′ | (SEQ ID No 36) |
| 3′ end, Ad5 bp 922-891: | |
| 5′-CGAGATCGATCACCTCCGGTACAAGGTTTGGCATAG-3′ | (SEQ ID No. 37) |

This amplified DNA fragment (sometimes hereinafter referred to as Fragment A) then was digested with XhoI and ClaI, which cleaves at the native ClaI site (bp 917), and ligated to the XhoI and ClaI sites of pSE280-E, thus reconstituting the 5′ end of the E1 region beginning 8 bp upstream of the ATG codon.

PCR then was performed to amplify Adenovirus 5 DNA from bp 3,323 through 4,090 contiguous with an EcoRI restriction site. The primers which were employed were as follows:

| | |
|---|---|
| 5′ end, Ad5 bp 3323-3360: | |
| 5′-CATGAAGATCTGGAAGGTGCTGAGGTACGATGAGACC-3′ | (SEQ ID No. 38); and |
| 3′ end, Ad5 bp 4090-4060: | |
| 5′-GCGACTTAAGCAGTCAGCTG-AGACAGCAAGACACTTGCTTGATCCAAATCC-3′ | (SEQ ID No.39). |

This amplified DNA fragment (sometimes hereinafter referred to as Fragment B) was digested with BgIII, thereby cutting at the Adenovirus 5 BgIII site (bp 3,382) and EcoRI, and ligated to the BgIII and EcoRI sites of pSE280-AE to reconstruct the complete E1a and E1b region from Adenovirus 5 bp 552 through 4,090. The resulting plasmid is designated pSE280-E1.

A construct containing the intact E1a/b region under the control of the synthetic promoter GRE5 was prepared as follows. The intact E1a/b region was excised from pSE280-E1, which was modified previously to contain a BamHI site 3′ to the E1 gene, by digesting with XhoI and BamHI. The XhoI to BamHI fragment containing the E1a/b fragment was cloned into the unique XhoI and BamHI sites of pGRE5-2/EBV (U.S. Biochemicals, Cleveland, Ohio) to form pGRE5-E1).

Bacterial transformants containing the final construct were identified. Plasmid DNA was prepared and purified by banding in CsTFA prior to use for transfection of cells.

Construction of Plasmid Including Adenovirus 5 E2A Sequence

The Adenovirus 5 genome was digested with BamHI and SpeI, which cut at bp 21,562 and 27,080, respectively. Fragments were separated on an agarose gel and the 5,518 bp BamHI to SpeI fragment was isolated. The 5,518 bp BamHI to SpeI fragment was digested further with SmaI, which cuts at bp 23,912. The resulting 2,350 bp BamHI to SmaI fragment was purified from an agarose gel, and ligated into the superlinker shuttle plasmid pSE280, and digested with BamHI and SmaI to form pSE280-E2 BamHI-SmaI.

PCR then was performed to amplify Adenovirus 5 DNA from the SmaI site at bp 23,912 through 24,730 contiguous with NheI and EcoRI restriction sites. The primers which were employed were as follows:

| | |
|---|---|
| 5′ end, Ad5 bp 24,732-24,708: | |
| 5′-CACGAATTCGTCAGCGCTTCTCGTCGCGTCCAAGACCC-3′ | (SEQ ID No. 40); |
| 3′ end, Ad5 bp 23,912-23,934: | |
| 5′-CACCCCGGGGAGGCGGCGGCGACGGGGACGGG-3′ | (SEQ ID No. 41) |

This amplified DNA fragment was digested with SmaI and EcoRI, and ligated to the SmaI and EcoRI sites of pSE280-E2 Bam-Sma to reconstruct the complete E2a region from Ad5 bp 24,730 through 21,562. The resulting construct is pSE280-E2a.

In order to convert the BamHI site at the 3′ end of E2a to a SalI site, the E2a region was excised from pSE280-E2a by cutting with BamHI and NheI, and recloned into the unique BamHI and NheI sites of pSE280. Subsequently, the E2a region was excised from this construction with NheI and SalI in order to clone into the NheI and SalI sites of the pMAMneo (Clonetech, Palo Alto, Calif.) multiple cloning site in a 5′ to 3′ orientation, respectively. The resulting construct is pMAMneo E2a.

Bacterial transformants containing the final pMAMneo-E2a were identified. Plasmid DNA was prepared and purified by banding in CsTFA. Circular plasmid DNA was linearized at the Xmnl site within the ampicillin resistance gene of pMAMneo-E2a, and further purified by the phenol/chloroform extraction and ethanol precipitation prior to use for transfection of cells.

Transfection and Selection of Cells

In general, this process involved the sequential introduction, by calcium phosphate precipitation, or other means of DNA delivery, of two plasmid constructions each with a different viral gene, into a single tissue culture cell. The cells were transfected with a first construct and selected for expression of the associated drug resistance gene to establish stable integrants. Individual cell clones were established and assayed for function of the introduced viral gene. Appropriate candidate clones then were transfected with a second construct including a second viral gene and a second selectable marker. Transfected cells then were selected to establish stable integrants of the second construct, and cell clones were established. Cell clones were assayed for functional expression of both viral genes.

A549 (ATCC Accession No. CCL-185) were used for transfection. Appropriate selection conditions were established for G418 and hygromycin B by standard kill curve determination.

Transfection of A549 Cells with Plasmids Including E1 and E2a Regions

pMAMNeo-E2a was linearized with Xmnl with the Amp^{R} gene, introduced into cells by transfection, and cells were selected for stable integration of this plasmid by G418 selection until drug resistant colonies arose. The clones were isolated and screened for E2a expression by staining for E2a protein with a polyclonal antiserum, and visualizing by immunofluorescence. E2a function was screened by complementation of the temperature-sensitive mutant Ad5ts125 virus which contains a temperature-sensitive mutation in the E2a gene. (Van Der Vliet, et al., J. Virology, Vol. 15, pgs. 348-354 (1975)). Positive clones expressing the E2a gene were identified and used for transfection with the 7 kb EcoRV to Xmnl fragment from pGRE5-E1, which contains the GRE5 promoted E1a/b region plus the hygromycin^{R} gene. Cells were selected for hygromycin resistance and assayed for E1a/b expression by staining with a monoclonal antibody for the E1 protein (Oncogene Sciences, Uniondale, N.Y.). E1 function was assayed by ability to complement an E1-deleted vector. At this point, expression and function of E2a was verified as described above, thus establishing the expression of E1a/b and E2a in the positive cell clones.

A transfected A549 (A549 (ATCC Accession No. CCL-185);) cell lines showed good E1a/b and E2a expression and was selected for further characterization. It was designated the S8 cell line.

G. Preparation of Adenoviral Vectors Containing Ad5.βgal.ΔF Genome in S8 Improved Fiber-Complementing Cell Lines

To prepare adenoviral vectors containing Ad5.βgal.ΔF (Ad5.βgal.ΔF has been was deposited the ATCC under accession number VR2636) in S8 cells containing alternative forms of TPL for enhancing the expression of fiber proteins, the protocol as described in Example 2 for preparing Ad5.βgal.ΔF in 211B cells was followed with the exception of pretreatment with 0.3 μM dexamethasone for 24 hours as described above. Thus, viral particles with the wildtype Ad5 fiber protein on their surface and containing the fiberless Ad5.βgal.ΔF genome were produced in 633 cells. Particles produced in 644 cells also contained the fiberless Ad5.βgal.ΔF genome, but had the chimeric 5T3H fiber protein, with the Ad3 fiber knob, on their surface.

Thus, these viral preparations, prepared as described herein are useful for targeting delivery of the Ad5.βgal.ΔF, Ad5.GFP.ΔF, or other similarly constructed fiberless genome with either wild-type or modified fibers. Preferably for purposes herein the fibers are from an Ad serotype D virus, more preferably from Ad37.

### EXAMPLE 6

Pseudotyping and Infectivity of Recombinant Adenoviral Vectors Produced with Improved Fiber-Complementing Cell Lines

A. Pseudotyping of Ad5.βgal.ΔF

To verify that adenoviral vectors were produced had altered tropisms, viral particles were purified from either 633 or 644 cells and were then Western blotted and probed with a polyclonal rabbit antibody against the Ad2 fiber (which detects the Ad5 and chimeric 5T3H fiber proteins).

B. Infectivity of Cells with 633 or 644 Generated Virus Particles

The cell lines, 633 or 644, prepared as described above, were infected with the indicated number of particles/cell of Ad5.βgal.ΔF and virus particles produced. Virus was then used to infect selected cell lines, including 211B, MRC-5 human fibroblasts, A-10 rat aortic endothelial cells, and THP-1 human monocytic cells. Unbound virus was removed by washing the cells and the cells were further incubated at 37° C. for 48 hours. Cells were then fixed with glutaraldehyde and stained with X-gal. The percentage of stained cells was then determined by light microscopy where all experiments were done in triplicate.

The results indicated that adenoviral vectors could be retargeted by pseudotyping using packaging cell lines expressing different fiber proteins. Particles containing either fiber were equally infectious on 211B cells, while MRC-5 fibroblasts and THP-1 cells were more readily infected by virus containing the chimeric fiber. The A-10 rat endothelial cells were more readily infected by particles containing the wildtype Ad5 fiber protein.

### EXAMPLE 7

Transient Transcomplementation

The ability of adenovirus type 5 (Ad5) to deliver therapeutic genes to cells is mediated by the interaction of the adenoviral fiber protein with the coxsackievirus-adenoviral receptor (CAR). Because a wide-range of cells express CAR, it was thought that it would be difficult to use adenoviruses to deliver genes to specific cell types. A system for testing modified fiber genes to identify tropisms of interest is described in copending U.S. application Ser. No. 09/482,682 (also filed as International PCT application No. PCT/US00/00265 on Jan. 14, 2000). An in vitro system has been developed that involves infection of tissue culture cells with a fiber-deleted Ad and transient co-transfection with a plasmid directing fiber expression. This system allows one to produce and evaluate modified fibers expressed on a viral particle. This system can be used to produce therapeutic quantities of adenoviral vectors with modified fiber proteins, with such fibers having a new tropism added by insertion of a desired ligand into the fiber gene. These fibers may also have the natural tropism (i.e. binding to CAR) ablated.

Plasmids used were pDV60 and pDV55 were prepared as described herein and in U.S. application Ser. No. 09/482,682 (also filed as International PCT application No. PCT/US00/00265 on Jan. 14, 2000). pDV60 is an pcDNA3.1-based expression plasmid that contains the CMV promoter, Ad5 tripartite leader, an intron, and the Ad5 fiber gene sequence. pDV55 contains no fiber gene and serves as the negative control. Ad5.βgal.ΔF and 211B are described above. 293T cells are identical to 293 cells except they express an integrated SV40 large T antigen gene. HDF cells are human diploid fibroblasts. 293T cells express CAR and αᵥ integrins; HDF cells express αᵥ integrins but no CAR. Transfections with fiber expression plasmids were performed with Lipofectamine (GIBCO-BRL) using 20 mg DNA and 50 ml Lipofectamine per 15 cm dish. Cells were maintained in DMEM supplemented with 10% fetal bovine serum.

The fiber deletion mutation of Ad5.βgal.ΔF is complemented in trans by passaging virions through 211B, a cell line that stably expresses functional Ad5 fiber. The present system was designed to complement Ad5.βgal.ΔF by modified fibers expressed from transfected episomal plasmids in 293T cells. The result is a simplified and rapid method to incorporate modified fibers on a viral particle containing the Ad5.βgal.ΔF genome that does not require propagation of the virus.

The feasibility of transcomplementation of Ad5.βgal.ΔF with episomal fiber-expressing plasmids was demonstrated in the following experiment. 293T cells were transfected with one of two plasmids: pDV55, which expresses no fiber or pDV60, which expresses wildtype Ad5 fiber. Fiber expression persists for at least six days. Twenty-four hours after transfection, these cells were infected at 2000 particles/cell with Ad5.βgal.ΔF passaged through 211B cells. Seventy-two hours later, a crude viral lysate (CVL) was generated by exposing the cells to five freeze-thaw cycles. Viral particles were purified by cesium chloride gradient centrifugation. The resulting virions incorporated the fiber expressed from the episomal plasmid, as confirmed by Western blots performed with an antibody specific to the Ad5 fiber.

Episomal plasmid transcomplementation system is suitable for quickly expressing and evaluating the properties of modified fibers in the context of a viral particle. Episomal plasmid transcomplementation will also be of great utility for quickly evaluating a bank of modified fibers for other binding properties, including new tropisms and the ablation of the native tropism. In addition to the rapid generation and testing of large numbers of modified fibers, there are other advantages to the Ad5.βgal.ΔF transcomplementation system in terms of production and safety. Episomal plasmid transcomplementation has the inherent advantage over transcomplementation in that it is not necessary to make a stable cell line for every modified fiber for complementation with Ad5.βgal.ΔF. Because the Ad5.βgal.ΔF is deleted in E1, E3 and fiber, there is an additional gene deletion, which should render it very suitable for gene therapy. In addition, the presence of the fiber gene deletion decreases the opportunity to generate replication-competent virus via recombination in the packaging cells. A single Ad vector preparation can be retargeted to any number of different cell types simply by transfecting the cells with the appropriate fiber-expression construct.

### EXAMPLE 8

Preparation of Adenoviral Gene Delivery Vectors Containing the Ad37 Fiber Protein

This example describes construction of packaging cell lines expressing the Ad37 fiber protein, and their use in generating particles of a fiber-deleted Ad vector (such as Ad5.βgal.ΔF) containing this fiber protein. The fiber protein is attached to the viral capsid by binding to the penton base protein through its N-terminus, and the Ad37 fiber was modified in order to make its N-terminal sequence more closely match that of the Ad5 protein to ensure that it would efficiently bind the Ad5 penton base in these vectors.

A. Materials and Methods

Cell lines and wild-type adenovirus. Human A549 lung carcinoma epithelial cells and human Chang C conjunctival cells (American Type Culture Collection) were maintained in complete Dulbecco's Modified Eagle Medium (DMEM) with 10% fetal bovine serum. Wild-type Ad19p and Ad37 (ATCC) were propagated in A549 cells and purified by banding on CsCl₂ density gradients as previously described (Huang et al. (1999) J. Virol. 73:2798-2802). Viral protein concentration was determined by the Bio-Rad Protein Assay, and was used to calculate the number of viral particles based on the known molecular weight of Ad2 virions (1 μg=4×10⁹ particles).

B. Construction of the Ad37 Fiber Expressing Cell Lines and the Recombinant Ad37 Knob Protein.

1. Construction of an Expression Plasmid for the Ad37 Fiber Protein (pDV80)

The plasmid designated pDV80 (see, SEQ ID No. 42) prepared for expression of the Ad37 fiber protein in mammalian cells, uses the same regulatory elements as the elements in pDV60, pDV67, and pDV69 to express the Ad37 fiber in packaging lines. It was constructed in two steps.

First, the Ad37 fiber open reading frame was amplified from Ad37 genomic DNA using synthetic oligonucleotide primers,

| | | |
|---|---|---|
| L37: | 5′ TGT CCT GGA TCC AAG ATG AAG CGC GCC CGC CCC AGC GAA GAT GAC TTC 3′ | (SEQ ID NO. 43) and |
| 37FR: | 5′ AAA CAC GGC GGC CGC TCT TTC ATT CTT G 3′ | SEQ ID NO. 44). |

L37 Contains nucleotides (underlined) that differ from the Ad37 genomic sequence in order to add a unique BamH1 site (bold) before the start codon (italicized) and to create point mutations that make the N-terminal sequence of the fiber more closely match the N-terminal sequence of the Ad5 fiber protein as follows:

| | | |
|---|---|---|
| Ad37 | MSKRLRVEDDFNPVYPY | (SEQ ID No. 45) |
| | ↓↓↓↓↓↓ | |
| | KRARPS | (SEQ ID No. 46) |
| Ad5 | MKRARPSEDTFNPVYPY | (SEQ ID No. 47). |

37FR also incorporates a unique Not1 site (bold). The PCR product was inserted into the BamH1 and Not1 sites of pCDNA3.1zeo(+) (Invitrogen) to create pDV78. The correct sequence of the Ad37 fiber protein, including inserted changes, was confirmed by sequencing.

Two point mutations in the fiber gene in the 705 line, S356 to P356 and I362 to T362, were discovered by the sequencing. The mutations are not in the receptor binding domain in Ad37 fiber gene in the 705 cell line. They are buried in the knob trimer interface. To confirm that the these mutations do not affect receptor binding, the Ad37 fiber protein with the correct sequence was recloned, and 293T cells transfected with the virus and subsequently infected with Ad5.GFPΔF to produce Ad37 pseudotyped virus. The results were the same as the results of the experiments with Ad37 pseudotyped virus produced from line 705 (see, Wu et al. (2001) Virology 279:78-89).

Second, a 1.2 kb Bam H1/Bgl II fragment containing an adenovirus type 5 tripartite leader was excised from pDV55 (see EXAMPLE 3) and inserted into the Bam H1 site of pDV78 to create pDV80 (SEQ ID No 42). Plasmid pDV80 has been deposited in the ATCC under accession number PTA-1147.

2. Construction of the Recombinant Ad37 Knob Protein

Recombinant Ad37 knob protein containing an N-terminal T7•Tag was produced in E. coli using the PET expression system (Novagen). Ad37 fiber DNA (GenBank accession number U69132) was PCR amplified from wild-type Ad37 genomic DNA using the following primers (SEQ ID Nos. 48 and 49):

5′ GGATCCATGGGATACTTGGTAGCA 3′ (BamHI site underlined and

5′ GCAACTCGAGTCATTCTTGGGCAATATAGG 3′(XhoI site underlined).

The PCR reactions were performed at 94° C. (denaturation), 55° C. (annealing), 72° C. (extension, 30 cycles) using Taq DNA polymerase (Qiagen). The amplified DNA fragments, which contained residues 172 to 365 of the Ad37 fiber protein with the addition of an N-terminal start codon (italicized), were purified and subcloned into the pCR-TOPO vector using the TA-Cloning Kit (Invitrogen). No replication errors were found by DNA sequencing. Plasmids from cultured transformed colonies were purified and digested with BamHI and XhoI. The fragment was inserted into the BamHI and XhoI sites of the bacterial expression vector, pET21a (Novagen), and transformed into (DE3)pLYS S expression cells (Invitrogen). Colonies were selected for knob expression by induction with 1 mM IPTG for four hours at 37° C. and knob expression was determined by SDS-PAGE. The colony displaying highest knob expression was used for large-scale knob expression and induced with 0.5 mM IPTG at 30° C. for four hours.

The recombinant T7•Tagged Ad37 knob protein was purified from sonicated bacteria using the T7•Tag Affinity Purification Kit as recommended by the manufacturer (Novagen). Recovered protein was analyzed for purity by SDS-PAGE followed by Coomassie staining or Western blotting with an HRP-conjugated α-T7•Tag monoclonal antibody as described by the manufacturer (Novagen) or an α-Ad37 fiber rabbit antibody.

3. Preparation of Cell Lines that Express the Ad37 Fiber Protein

Plasmid pDV80 DNA was purified using the Qiagen method and electroporated into the adenovirus-complementing cell line E1-2a S8 (see Examples herein; see also, Gorziglia et al. (1996) J. Virology 70:4173-4178; and Von Seggern et al. (1998) J. Gen. Virol. 79:1461-1468). Stable clones were selected with 600 μg/ml zeocin (Invitrogen).

Clones were expanded and were screened for fiber expression by indirect immunofluorescence (Von Seggern et al. (1998) J. Gen. Virol. 79:1461-1468) using a rabbit polyclonal antibody directed against the Ad37 fiber (α-Ad37 fiber rabbit antibody) raised by immunizing rabbits with recombinant Ad37 fiber protein. Two clones (lines 705 and 731) that expressed the protein at a uniformly high level were selected.

### EXAMPLE 9

Production of Pseudotyped Ad Vector Particles

To generate vector particles equipped (‘pseudotyped’) with the Ad37 fiber protein, the Ad37 fiber-expressing 705 cells were infected (approximately 1000 particles/cell) with Ad5.βgal.ΔF or with Ad5.GFP.ΔF.

Materials and Methods

Ad5.βgal.ΔF

The construction of Ad5.βgal.ΔF is described in Example 2 (it has been deposited on Jan. 15, 1999, with the ATCC as listed above under accession number VR2636; see also, Von Seggern et al. (1999) J. Virol. 73:1601-1608; copending U.S. application Ser. No. 091482,682 filed Jan. 14, 2000, and also International PCT application No. PCT/US00/00265, filed Jan. 14, 2000).

Ad5.GFP.ΔF

Ad5.GFP.ΔF was constructed by recombination in bacteria using a modification of the AdEasy System (see, U.S. Pat. No. 5,922,576; see, also He et al. (1998) Proc. Natl. Acad. Sci. U.S.A. 95:2509-2514; the system is publicly available from the authors and other sources).

First, a fiber-deleted genomic plasmid was constructed by removing the fiber gene from pAdEasy-1 (see, U.S. Pat. No. 5,922,576; and He et al. (1998) Proc. Natl. Acad. Sci. U.S.A. 95:2509-2514; the AdEasy system and vectors are publicly available from He et al. at Johns Hopkins University). Plasmid pAdEasy-1 contains the entire Ad5 genome, except for nucleotides 1-3,533, which encompass the E1 genes, and nucleotides 28, 130-30,820, which encompass the E3 gene.

Plasmid pDV43 (see Example 2; see, also Von Seggern et al. (1999) J. Virol. 73:1601-1608) was digested with Pac1, the ends blunted by treatment with the large fragment of E. coli DNA polymerase and dNTPs, and the product re-ligated to produce plasmd pDV76. The resulting plasmid pDV76 is identical to pDV43 except for loss of the Pac1 site and contains the right end of the Ad5 genome with E3 and fiber deletions. A 4.23 kb fragment from PDV76 was amplified using the oligonucleotide primers (SEQ ID Nos. 50 and 51:

5′ CGC GCT GAC TCT TAA GGA CTA GTT TC 3′, including the unique Spe1 site in the Ad5 genome (bold); and 5′ GCG CTT AAT TAA CAT CAT CAA TAA TAT ACC TTA TTT T 3′, including a new Pac1 site (bold) adjacent to the right Ad5 ITR. Hence the resulting PCR amplified fragment contains nucleotides 27,082 to 35,935 of the Ad5 genome with deletions of nucleotides 28,133 to 32,743 (the E3 and fiber genes), and was used to replace the corresponding Spe1/Pac1 fragment of pAdEasy 1 (see, U.S. Pat. No. 5,922,576) to create pDV77.

Second, E. coli strain BJ5183 was electroporated with a mixture of pDV77 and Pme1-linearized pAdTrack as described (U.S. Pat. No. 5,922,576; He et al. (1998) Proc. Natl. Acad. Sci. U.S.A. 95:2509-2514), and DNA was isolated from kanamycin-resistant colonies. The resulting plasmid, pDV83, contains a complete Ad5 genome with E1-, E3-, and fiber-deletions with a CMV-driven GFP reporter gene inserted at the site of the E1 deletion. The full length Ad chromosome was isolated by Pac1 digestion, and transfected into the E1- and fiber-complementing 633 cells (Von Seggern et al. (2000) J. Virol. 74:354-362). The 633 cells were produced by electroporating pDV67 (SEQ ID No. 30, deposited under ATCC accession number PTA-1145) into the E1-2a S8 cells, described above. The recovered virus Ad5.GFP.ΔF was then plaque purified by plating on 633 cells and virus stocks were prepared by freeze-thawing cell pellets.

Ad5-pseudotype Particle Production

Particles with Ad5 Fiber

Ad5-pseudotyped particles were generated by virus growth in 633 cells, which express the wild type Ad5 fiber protein. Viral particles were isolated and purified over CsCl gradients (Von Seggern et al. (1999) J. Virol. 73:1601-1608; purified by centrifugation on preformed 15-40% CsCl gradients (111,000×g for three hours at 4° C.)). For analysis of viral proteins, ten μg of the purified particles were electrophoresed on 8-16% gradient gels and the protein transferred to nylon membranes. The resulting blot was probed with rabbit polyclonal antibodies raised against recombinant Ad37 fiber or Ad5 fiber or penton base proteins expressed in baculovirus-infected cells.

Particles with Ad37 Fiber

Cells from the Ad37 fiber producing cell line 705 were infected at approximately 1000 particles/cell with Ad5.βgal.ΔF or with Ad5.GFP.ΔF. Viral particles were isolated and purified over CsCl gradients. The bands were harvested, dialyzed into storage buffer (10 mM Tris-pH 8.1, 0.9% NaCl, and 10% glycerol), aliquoted and stored at −70° C.

Viral Protein Analyses

For analysis of viral proteins, 10 μg of purified Ad5.βgal.ΔF particles with no fiber (grown in 293 cells), the Ad5 fiber (grown in 633 cells), or the Ad37 fiber (grown in 705 cells) were electrophoresed by 8-16% polyacrylamide gradient SDS-PAGE and the proteins were transferred to nylon membranes. The blot was then probed with α-Ad37 fiber rabbit antibody. Ad5 fiber and penton base were detected by reprobing the blot with polyclonal antibodies raised against recombinant proteins expressed in baculovirus-infected cells (Wickman et al. (1993) Cell 2:309-319).

Adenovirus Infection and Cell Binding Assays

Adherent Chang C and A549 cells were infected with GFP expressing Ad5 vectors containing the Ad5 fiber (Ad5.GFP.ΔF/5F) or the Ad37 fiber (Ad5.GFP.ΔF/37F) at 10,000 particles per cell for 3 hours at 37° C., 5% CO₂ in DMEM, 10% FCS. Cells were washed twice with saline and then cultured overnight at 37° C., 5% CO₂. The next day, the cells were detached with buffer containing 0.05% (w/v) trypsin and 0.5 mM EDTA (Boehringer Mannheim) for 5 minutes at 37° C. Suspended cells were washed once with PBS and then resuspended in phosphate-buffered saline (PBS), pH 7.4. GFP fluorescence was measured with a FACScan flow cytometer. A threshold established by the fluorescence of uninfected cells was used to distinguish cells expressing GFP. To assess the role of CAR in Ad infection, 10,000 attached cells were pre-incubated with 180 μg/ml RmcB, a function-blocking anti-CAR monoclonal antibody (Hsu et al. (1988) J. Virol. 62:1647-1652), in complete DMEM for 1 hour at 4° C. A small volume containing Ad5.GFP.ΔF/5F or Ad5.GFP.ΔF/37F was then added at 10,000 particles per cell. The cells were infected for 3 hours, cultured overnight, harvested, and analyzed for GFP expression. Percent cells expressing GFP was determined by the percent of cells detected above a threshold set by the fluorescence of uninfected Chang C cells.

To measure adenovirus binding to cells, wild type Ad37 was labeled with ¹²⁵I using lodogen (Pierce) according to manufacturer instructions and separated from free ¹²⁵I by gel filtration as described (Huang et al. (1999) J. Virol. 73:2798-2802). Binding of radiolabeled wild type Ad37 on Chang C cells was then quantitated as described (Huang et al. (1999) J. Virol. 73:2798-2802). Non-specific binding was determined by incubating cells and labeled Ad37 particles in the presence of 100-fold concentration of unlabeled Ad37. Specific binding was calculated by subtracting the non-specific binding from the total cpm bound. To examine if divalent cations are required for binding, 10 mM ethylenediaminetetraacetic acid (EDTA) or various concentrations of CaCl₂, or MgCl₂ were added to cells before incubation with labeled virus. To examine if the receptor for Ad37 is a protein, cells were pretreated with 10 μg/ml trypsin (GIBCO), subtilisin (Sigma), proteinase K (Boehringer-Mannheim), and bromelain (Sigman) at 37° C. for 1 hour, then washed twice with complete DMEM before adding labeled virus. Cells were >95% viable after protease treatment.

Ad37 binding to conjunctival cells is calcium-dependent. Specific ¹²⁵I-labeled Ad37 binding to Chang C cells was measured in the presence of 10 mM EDTA and in the presence of varying concentrations of calcium chloride or magnesium chloride. Specific binding was determined by subtracting the nonspecific counts in the presence of 100-fold excess unlabeled virus from the total counts.

Pretreatment of conjunctival cells with proteases inhibits Ad37 binding. Change C cells were pretreated with various proteases for 1 hour before binding ¹²⁵I-labeled Ad37 to the cells. Nonspecific binding was measured by adding 100-fold unlabeled Ad37 to cells with ¹²⁵I-labeled Ad37 and subtracting from total counts for specific binding. Percent inhibition represents the difference in specific binding of untreated cells and pretreated cells as a percentage of the specific binding of untreated cells.

Virus Overlay Protein Blot Assay (VOPBA)

For VOPBA of human conjunctival membrane proteins probed with Ad37 in the presence of EDTA or calcium chloride, Chang C membrane fractions were separated by 8% SDS-PAGE and transferred to a PDVF membrane. The membrane was subsequently probed with or without whole Ad37 particles, a polyclonal antibody against Ad37 fiber, and finally a horseradish peroxidase conjugated anti-rabbit antibody, in the presence of EDTA or calcium chloride. Transferred Chang C membrane proteins were probed with recombinant Ad37 knob protein, instead of Ad37 knob, in the presence of calcium chloride.

Confluent monolayers of Chang C and A549 cells were detached by scraping, pelleted by centrifugation, and then resuspended in 250 mM sucrose, 20 mM HEPES, pH 7.0, 1 mM EDTA, and 2 μg/ml aprotinin and leupeptin. Cells were transferred into a dounce homogenizer and disrupted with 30 strokes. Organelles and nuclei were pelleted at 500 g for 15 min. Plasma membrane fragments were then pelleted from the supernatant of cell lysates at 200,000 g for 1 hour and then resuspended in 10 mM Tris•Cl, pH 8.1, 10 μg/ml aprotinin and leupeptin.

Cell membranes of Chang C or A549 cells were incubated (1:1) with a 2% SDS, non-reducing buffer and separated on an 8% polyacrylamide gel without boiling. Membrane proteins were then electroblotted onto a PVDF membrane (Immobilon-P) and blocked in 5% (w/v) milk in PBS, pH 7.4, 0.02% Tween-20 (PBS-T). After blocking, the membrane was incubated with 1 μg/ml wild-type Ad19p or Ad37 in 0.5% (w/v) milk in PBS-T, 1 mM CaCl₂, for 1 hour at room temperature. The membrane was then washed once with phosphate-buffered saline, pH 7.4 (PBS), 1 mM CaCl₂, and incubated with 1:500 dilution of α-Ad37 fiber rabbit antibody in 0.5% (w/v) milk in PBS-T, 1 mM CaCl₂, for 30 minutes at room temperature. The membrane was washed again with PBS, 1 mM CaCl₂, and incubated with 1:5000 dilution of horseradish peroxidase (HRP) conjugated α-rabbit antibody (Sigma) in 0.5% (w/v) milk in PBS-T, 1 mM CaCl₂, for 30 minutes at room temperature. The membrane was washed four times in PBS, 1 mM CaCl₂, once with PBS-T, 1 mM CaCl₂, and once in 1 mM CaCl₂. The blot was developed with enhanced chemiluminescence reagents (Pierce) for 5 minutes and placed onto a piece of Biomax film (Kodak) for 5 seconds to 1 minute. For divalent metal cation experiments, membranes were incubated in the presence of 2 mM EDTA instead of 1 mM CaCl₂ in all solutions. To assay fiber knob binding to cell membrane proteins, membrane filters were incubated with 1 μg/ml purified T7-tagged Ad37 knob protein in Tris-buffered saline, 0.1% Tween-20, 1 mM CaCl₂, for 1 hour at room temperature. α-Ad37 fiber rabbit antibody and HRP-conjugated anti-rabbit antibody were applied and the membrane was developed with substrate solution as described above.

Results: Comparison of Adenovirus Infection of Human Conjunctival and Lung Epithelial Cells with Virus Particles Retargeted with Ad5 or Ad37 Fiber Proteins

Packaging cell lines producing the Ad37 fiber protein were generated. Since the N-terminal amino acid sequences of the Ad5 and Ad37 fiber proteins differ significantly, and to ensure that the Ad37 fiber would be efficiently incorporated into Ad5 vector particles, several residues in the wild-type Ad37 fiber were mutated to more closely match the Ad5 sequence. Stable cell lines producing this fiber under control of the CMV promoter and the adenovirus type 5 tripartite leader were then generated and screened for fiber expression by indirect immunofluorescence. One clone (line 705), which expressed the Ad37 fiber at a high level, was selected for further study.

Cells from one cell line 633, which expresses the wild-type Ad5 fiber protein, and line 705 were infected with a fiber-deleted Ad5 vector carrying a βgalactosidase reporter gene. The resulting vector particles contained the Ad5 fiber protein (Ad5.βgal.ΔF/5F) and the Ad37 fiber protein (Ad5.βgal.ΔF/37F), respectively. Incorporation of the correct fiber protein into viral particles was verified by Western blotting. Adenoviral vectors containing the GFP reporter gene, Ad5.GFP.ΔF/5F and Ad5.GFP.ΔF/37F, were created in the same fashion.

Infection of a variety of cell types using the retargeted adenovirus particles was examined. As assayed by GFP fluorescence, Ad5.GFP.ΔF/5F exhibited good gene delivery to lung epithelial (A549) and conjunctival cells (Chang C). In contrast, Ad5.GFP.ΔF/37F efficiently delivered GFP to Chang C cells, but exhibited very poor gene delivery to A549 cells. Although CAR is expressed on the surface of A549 cells, as indicated by AD5.GFP.ΔF/5F infection, Ad5.GFP.ΔF/37F was unable to infect these cells efficiently. This experiment shows that the Ad37 fiber protein can confer preferential infection of human conjunctival cells, but not CAR-expressing human lung epithelial cells.

Hence CAR is not the primary receptor for Ad37. Recent studies reported that expression of CAR on the surface of chinese hamster ovary (CHO) cells did not improve Ad37 binding (Arnberg et al. (2000) J. Virol. 74:42-48), implying that Ad37 does not use CAR as a primary receptor. In order to verify this on human conjunctival cells, A549 and Chang C cells were pretreated with RmcB (Hsu et al. (1988) J. Virol. 62:1647-1652), a function-blocking monoclonal antibody against CAR. The RmcB antibody inhibited infection of A549 cells by Ad5.GFP.ΔF/5F, but it had little effect on infection of Chang C cells by Ad5.GFP.ΔF/37F. This indicates that CAR is not the primary receptor for Ad37 on Chang C conjunctival cells.

Ad37 binding to conjunctival cells requires divalent metal cations. It has been proposed (Roelvink et al. (1998) J. Virol. 72:7909-7915) that a combination of fiber binding to CAR and penton base binding to αᵥ-integrins allows some adenovirus serotypes to attach to cells. Although αᵥ-integrin binding to the RGD motif of the adenovirus penton base is of relatively low affinity (Wickman et al. (1993) Cell 2:309-319), it may nonetheless contribute to viral attachment to the cell surface. Ad37 shows a particularly strong affinity for binding to integrin αᵥβ₅ (Mathias et al. (1998) J. Virol. 72:8669-8675), suggesting that integrin αᵥβ₅ might be a primary receptor for Ad37. Binding of the RGD motif by αᵥ-integrins requires the presence of divalent cations, such as calcium or magnesium (Stuiver et al. (1996) J. Cell Physiol. 168:521-531). In contrast, no divalent cations were required for binding in the CAR-Ad12 knob complex (Bewley et al. (1999) Science 286:1579-1583).

To investigate the potential role of αᵥ-integrins and divalent metal cations in Ad37 receptor binding, ¹²⁵I-labeled Ad37 binding to Chang C cells was examined in the absence or presence of EDTA. EDTA inhibited Ad37 binding to conjunctival cells but did not alter Ad5 binding. These findings suggest a requirement for divalent metals for Ad37 binding. The presence of either calcium or magnesium ions helps αᵥβ₅ organize in focal contacts (Stuiver et al. (1996) J. Cell Physiol. 168:521-531), suggesting that calcium and magnesium aid in integrin αᵥβ₅ function. To further test the potential role of integrin αᵥβ₅ in Ad37 cell attachment, ¹²⁵I-labeled Ad37 binding to Chang C cells was measured in the presence of varying concentrations of calcium or magnesium chloride. Magnesium ions had little effect on Ad37 binding to Chang C cells. In contrast, calcium ions dramatically enhanced Ad37 binding to Chang C cells. The optimal concentration of calcium chloride for Ad37 binding was 1 mM, while higher concentrations of calcium actually decreased virus binding to cells. The fact that calcium, but not magnesium, promoted Ad37 attachment is not consistent with integrin αᵥβ₅ as the primary receptor for viral attachment to the cells since either metal will support ligand binding to integrin αᵥβ₅. Moreover, A549 cells express abundant αᵥ-integrins (Mathias et al. (1998) J. Virol. 72:8669-8675) but were unable to support efficient binding of Ad37.

Wild-type Ad37 particles bind to three conjunctival membrane proteins. Recent studies reported that protease treatment of CHO cells abolished Ad37 binding (Arnberg et al. (2000) J. Virol. 74:42-48), implying that Ad37 bound to a protein receptor on CHO cells. Scatchard analysis of Ad37 binding to Chang C cells showed that each cell expresses approximately 24,000 fiber binding sites (Huang et al. (1999) J. Virol. 73:2798-2802). To determine if the Ad37 binding site on human conjunctival cells is also a protein, Chang C cells were treated with different proteases prior to measuring binding of ¹²⁵I-labeled Ad37. Digestion of surface proteins by all four proteases inhibited Ad37 binding to Chang C cells by greater than 50%. This finding showed that Ad37 also binds to a protein receptor on Chang C cells.

Virus overlay protein blot assays (VOPBAs) were used to identify candidate viral protein receptors. This Western blot technique uses intact viral particles in place of antibodies to probe viral-receptors interactions. VOPBA was used herein to identify Chang C membrane proteins that bind to Ad37. In the absence of Ad37 particles, no protein bands were observed, while addition of virus in the absence of calcium revealed binding to a single 45 kDa protein. In the presence of 1 mM calcium chloride, Ad37 reacted with three proteins with approximate molecular weights of 45, 50 and 60 kDa. The same three proteins were detected using a recombinant Ad37 fiber knob alone, indicating that Ad37-receptor interactions are fiber mediated and do not involve interactions of other capsid proteins such as the penton base. The size of the calcium-independent protein (45 kDa) is very similar to the known molecular weight of CAR. A direct comparison of the Ad37 VOPBA and a CAR Western blot showed that the 45 kDa receptor co-migrates with CAR on SDS-PAGE. Moreover, two other members of subgroup D adenoviruses, Ad9 and AD15, have been shown to bind to CAR (Roelvink et al. (1998) J. Virol. 72:7909-7915).

Since CAR does not appear to mediate Ad37 binding on intact Chang C cells, the possibility that the 50 or 60 kDa protein serves this function was tested by examining an adenovirus serotype that does not bind to Chang C cells. Ad19p, a closely related subgroup D adenovirus, binds poorly to Chang C cells (Huang et al. (1999) J. Virol. 73:2798-2802) and Ad19p recognition of the Ad37 receptor is therefore unlikely. Ad19p particles bound to the 45 and 60 kDa receptors in the VOPBA, but did not bind to the 50 kDa receptor. Moreover, the 50 kDa receptor is expressed on Chang C cells, but not A549 cells, which only support low levels of Ad37 binding and infection. Taken together, these data indicate that the 50 kDa protein is a primary candidate receptor for Ad37 on human conjunctival cells.

Discussion

The identification of the CAR protein as a major adenovirus receptor does not explain why certain subgroup D members, such as Ad37, preferentially infect ocular cells. A 50 kDa human conjunctival cell membrane protein is identified herein as a primary candidate for the receptor for Ad37. This 50 kDa protein is not present on A549 lung epithelial cells. Ad37 binding to this receptor is calcium-dependent, which is consistent with Ad37 binding and infection experiments. Ad37 also bound to a 60 kDa protein that is present on human conjunctival and lung epithelial cells. It does not, however, appear to be serotype specific. The molecular weights of MHC class I heavy chain, which has been proposed as a receptor for Ad5, and αᵥβ₃ and αᵥβ₅ intergrins, receptors for the penton base, are distinct from the 50 or to kDa receptor characterized in this study.

The studies of Ad37-receptor interaction using VOPBAs are consistent with previous studies showing that subgroup D adenoviruses can bind to the extracellular domain of CAR (Roelvink et al. (1998) J. Virol. 72:7909-7915). Biochemical and structural studies on knob-CAR interactions indicate that the CAR binding site is located on the AB-loop of the fiber knob. Alignment of the fiber sequences of Ad37 and other adenoviruses reveals that the AB-loop of Ad37 is similar to those of Ad12 and Ad5. Moreover, a phylogenetic tree of adenovirus knobs (Roelvink et al. (1998) J. Virol. 72:7909-7915) shows that fiber proteins of subgroup D are similar to those of subgroup C and E, which use CAR as their primary receptor. Ad37 does not, however, appear to effectively use CAR as a primary receptor, as demonstrated by virus binding and infection studies on Chang C conjunctival cells and A549 lung epithelial cells.

It has been reported that Ad37 uses sialic acid as a receptor on chinese hamster ovary (CHO) cells and human lung carcinoma (A549) cells (Arnberg et al. ((2000) J. Virol. 74:42-48). Human conjunctival cells were not studied. Human corneal epithelial (HCE) cells were the only ocular cell line studied and Ad37 binds relatively poorly to these cells, compared to binding on A549 cells (Arnberg et al. ((2000) J. Virol. 74:42-48). In addition, 8,4×10⁷ wheat germ agglutinin molecules per cell were required to significantly inhibit Ad37 binding to sialic acid on sialic acid positive CHO cells (Arnberg et al. (2000) J. Virol. 74:42-48), three orders of magnitude higher than the number of Ad37 receptors on Chang C conjunctival cells (Huang et al. (1999) J. Virol. 73:2798-2802). Clearly, sialic acid is not the only factor responsible for Ad37 binding to the cell surface and its influence on Ad37 tropism is unclear.

The results herein show that Ad37 selects a 50 kDa cellular receptor for binding to conjunctival cells, but it is possible that sialic acid also plays a role in this interaction. The characterization and identification of the Ad37 receptor have therapeutic implications and also explain the different tropism of Ad37. The 50 kDA receptor for Ad37 may also be the receptor for other subgroup D adenoviruses that cause severe cases of EKC, Ad19a and Ad8. Ad19p is a nonpathogenic variant of Ad19 (Arnberg et al. (1998) Virology 22 7:239-244) while Ad19A, along with Ad8 and Ad37, are major causes of EKC. Ad19a and Ad37 have identical fiber proteins (Arnberg et al. (1998) Virology 227:239-244) and have similar tropism in vivo. Ad8, Ad19a, and Ad37 agglutinate dog and guinea pig erythrocytes more effectively than four other serotypes that are associated with less severe forms of conjunctivitis (Arnberg et al. (1998) Virology 227:239-244), implying that the receptors of Ad18, Ad19A, and Ad37 have similar characteristics. Hence, this 50 kDa receptor is an attractive drug target against EKC caused by adenoviruses to provide therapeutic intervention of ocular diseases associated with these viruses.

### EXAMPLE 10

Targeting of the Ad5 Vector to Photoreceptor Cells

The fiber-deleted adenovirus vector Ad5.GFP.ΔF was propagated in 705 cells, which express a modified Ad37 fiber protein. Viral particles (Ad5.GFP.Δf/37F) were harvested, CsCl-purified and dialized into 0.9% NaCl, 10 mM Tris, pH 8.1, and 10% glycerol. Two to three μl of the resulting solution, containing approximately 1×10⁹ particles/μl was injected into the vitreous chamber of a mouse eye. Seven days post-injection, eyes were harvested, fixed with paraformaldehyde and cryo-sectioned. Sections were stained with an anti-rhodopsin antibody to identify photoreceptor cells and with DAPI to show all cell nuclei. The resulting sections showed red anti-rhodopsin staining in the photoreceptors, blue DAPI-stained nuclei, and green GFP staining in any transduced cells. The results revealed substantially exclusive transduction of photoreceptors. Co-localization of rhodopsin staining and GFP expression indicated selective transduction of photoreceptor cells.

As a control, contralateral eyes were injected with a stock of the fiber-deleted vector AD5.βgal.ΔF grown in the same Ad37 fiber-expressing cells. Since this virus (Ad5.βgal.ΔF/37F) produces βgal rather than GFP, the green staining is absent from the photoreceptors.

Additional experiments using the AD37 fiber for targeting to the photoreceptor cells have been performed. Subretinal and intravitreal injection have been used in mouse models and the results demonstrate targeting to the photoreceptors. As with intravitreally injected eyes, the major cell type infectd via subretinal administration was the photoreceptor.

As noted, Ad5.GFP.ΔF/37F infected Chang C cells efficiently, but A549 cells poorly. Ad37 fiber protein confers preferential infection on human conjunctival cells, but not CAR-expressing human lung epithelial cells. Binding to conjunctival cells requires divalent cations.

Since modifications will be apparent to those of skill in this art, it is intended that this invention be limited only by the scope of the appended claims.

What is claimed is:

1. An isolated nucleic acid molecule, comprising:
adenovirus inverted terminal repeat sequences; an adenovirus packaging signal operatively linked thereto; and a photoreceptor-specific promoter.

2. The isolated nucleic acid molecule of claim 1, further comprising a nucleic acid encoding a therapeutic product operatively linked to the promoter.

3. The isolated nucleic acid molecule of claim 1, wherein the promoter is a rhodopsin promoter.

4. The nucleic acid molecule of claim 1, wherein the adenovirus genome does not encode a functional fiber protein such that packaging the nucleic acid requires complementation in a packaging cell.

5. A recombinant adenovirus vector, comprising the nucleic acid molecule of claim 1 packaged therein.

6. A recombinant adenovirus vector of claim 5, wherein inverted terminal repeat sequences (ITR) and a packaging signal are derived from adenovirus type 2 or adenovirus type 5.

7. A recombinant adenovirus vector of claim 5, wherein the virus comprises a fiber protein.

8. A recombinant adenovirus vector of claim 7, wherein the fiber protein selectively binds to photoreceptors in the eye of a mammal.

9. A recombinant adenovirus vector of claim 7, wherein the fiber is a chimera composed of N-terminal sequences from adenovirus type 2 or type 5, and a sufficient portion of an adenovirus serotype D fiber for selective binding to photoreceptors in the eye of a mammal.

10. A method for targeted delivery of a gene product to the eye of a mammal, comprising:
administering a recombinant adenovirus virus that comprises heterologous DNA encoding the gene product or resulting in expression of the gene product, wherein the recombinant virus comprises a fiber protein that specifically or selectively binds to receptors that are expressed on cells in the eye.

11. The method of claim 10, wherein the cells are photoreceptors.

12. The method of claim 10, wherein administration is effected by intraocular delivery.

13. The method of claim 10, wherein administration is effected by a method selected from subretinal injection, intravenous administration, periorbital administration, and intravitreal administration.

14. The method of claim 10, wherein the recombinant virus comprises a fiber protein from an adenovirus type D serotype.

15. The method of claim 10, wherein the fiber protein is an adenovirus type 37.

16. The method of claim 10, wherein the fiber is a chimeric protein containing a sufficient portion of the N-terminus of an adenovirus type 2 or type 5 fiber protein for interaction with an adenovirus type 2 or type 5 penton, and a sufficient portion of an adenovirus serotype D knob portion of the fiber for selective binding to photoreceptors in the eye of a mammal.

17. The method of claim 10, wherein the recombinant virus is an adenovirus type D serotype.

18. The method of claim 10, wherein the encapsulated nucleic acid comprises a photoreceptor-specific promoter operatively linked to a nucleic acid comprising the therapeutic product.

19. The method of claim 18, wherein the therapeutic product is selected from the group consisting of a trophic factor, an anti-apoptotic factor, a gene encoding a rhodopsin protein, a wild-type Stargardt disease gene (STDG1), an anti-cancer agent and a protein that regulates expression of a photoreceptor-specific gene product.

20. The method of claim 10, wherein delivery is effected for treatment of an ocular disease.

21. The method of claim 20, wherein the disorder is a retinal degenerative disease.

22. The method of claim 20, wherein the disease is retinitis pigmentosa, Stargardt's disease, diabetic retinopathies, retinal vascularization, or retinoblastoma.

23. The method of claim 10, wherein the mammal is a human.

24. The method of claim 10, wherein the viral nucleic acid comprises:
an adenovirus inverted terminal repeat (ITR) sequences; and
an adenovirus packaging signal operatively linked thereto.

25. The method of claim 24, wherein the ITRs and packaging signal are derived from an adenovirus serotype B or C.

26. The method of claim 24, wherein the ITRs and packaging signal are derived from an adenovirus type 2 or 5.

27. The method of claim 24, wherein the viral nucleic acid further comprises a photoreceptor-specific promoter.

28. A method of targeted gene therapy, comprising:
administering a recombinant viral vector that comprises an adenovirus type 37 fiber protein or portion thereof, whereby the vector selectively transduces photoreceptors and delivers a gene product encoded by the recombinant viral vector; wherein the portion is sufficient for selective binding to photoreceptors.

29. The method of claim 28, wherein the vector is administered into the eye.

30. The method of claim 28, wherein the vector is administered to the vitreous cavity of the eye.

31. The method of claim 28, wherein administration is effected by subretinal injection, intravenous administration, periorbital administration or intravitreal administration.

32. The method of claim 10, wherein at least about 10⁷ plaque forming units of virus are administered.

33. The method of claim 31, wherein about 1 plaque forming unit to about 10¹⁴ plaque forming units of virus are administered.


SEQUENCE LISTING (50 sequences)

SEQ ID NO: 1 - DNA, 30 bp, Artificial Sequence
  Description of Artificial Sequence primer
cggtacacagaattcaggagacacaactcc

SEQ ID NO: 2 - DNA, 35 bp, Artificial Sequence
  Description of Artificial Sequence primer
gcctggatccgggaagttacgtaacgtgggaaaac

SEQ ID NO: 3 - DNA, 12 bp, Artificial Sequence
  Description of Artificial Sequence linker
cgcggatccgcg

SEQ ID NO: 4 - DNA, 8710 bp, Artificial Sequence
  Description of Artificial Sequence plasmid
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccggatccgcggcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcccgggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgcccagtgccgtcgaccggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagccggatcataatcagccataccacatttgtagaggttttacttgctttaaaaaacctccccacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 5 - DNA, 30 bp, Artificial Sequence
  Description of Artificial Sequence primer
atgggatccaagatgaagcgcgcaagaccg

SEQ ID NO: 6 - DNA, 30 bp, Artificial Sequence
  Description of Artificial Sequence primer
cataacgcggccgcttctttattcttgggc

SEQ ID NO: 7 - DNA, 7148 bp, Artificial Sequence
  Description of Artificial Sequence plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 8 - DNA, 7469 bp, Artificial Sequence
  Description of Artificial Sequence plasmid
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatctgaattcgagctcgctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 9 - DNA, 28 bp, Artificial Sequence
  Description of Artificial Sequence primer
tgcttaagcggccgcgaaggagaagtcc

SEQ ID NO: 10 - DNA, 23 bp, Artificial Sequence
  Description of Artificial Sequence primer
ccgagctagcgactgaaaatgag

SEQ ID NO: 11 - DNA, 23 bp, Artificial Sequence
  Description of Artificial Sequence primer
cctctcgagagacagcaagacac

SEQ ID NO: 12 - DNA, 11152 bp, Artificial Sequence
  Description of Artificial Sequence plasmid
aagcttgggcagaaatggttgaactcccgagagtgtcctacacctaggggagaagcagccaaggggttgtttcccaccaaggacgacccgtctgcgcacaaacggatgagcccatcagacaaagacatattcattctctgctgcaaacttggcatagctctgctttgcctggggctattgggggaagttgcggttcgtgctcgcagggctctcacccttgactcttttaatagctcttctgtgcaagattacaatctaaacaattcggagaactcgaccttcctcctgaggcaaggaccacagccaacttcctcttacaagccgcatcgattttgtccttcagaaatagaaataagaatgcttgctaaaaattatatttttaccaataagaccaatccaataggtagattattagttactatgttaagaaatgaatcattatcttttagtactatttttactcaaattcagaagttagaaatgggaatagaaaatagaaagagacgctcaacctcaattgaagaacaggtgcaaggactattgaccacaggcctagaagtaaaaaagggaaaaaagagtgtttttgtcaaaataggagacaggtggtggcaaccagggacttataggggaccttacatctacagaccaacagatgcccccttaccatatacaggaagatatgacttaaattgggataggtgggttacagtcaatggctataaagtgttatatagatccctcccttttcgtgaaagactcgccagagctagacctccttggtgtatgttgtctcaagaagaaaaagacgacatgaaacaacaggtacatgattatatttatctaggaacaggaatgcacttttggggaaagattttccataccaaggaggggacagtggctggactaatagaacattattctgcaaaaactcatggcatgagttattatgaatagcctttattggcccaaccttgcggttcccagggcttaagtaagtttttggttacaaactgttcttaaaacgaggatgtgagacaagtggtttcctgacttggtttggtatcaaaggttctgatctgagctctgagtgttctattttcctatgttcttttggaatttatccaaatcttatgtaaatgcttatgtaaaccaagatataaaagagtgctgattttttgagtaaacttgcaacagtcctaacattcacctcttgtgtgtttgtgtctgttcgccatcccgtctccgctcgtcacttatccttcactttccagagggtccccccgcagaccccggcgaccctcaggtcggccgactgcggcagctggcgcccgaacagggaccctcggataagtgacccttgtctctatttctactatttggtgtttgtcttgtattgtctctttcttgtctggctatcatcacaagagcggaacggactcaccatagggaccaagctagcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctggtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagc

SEQ ID NO: 13 - DNA, 19 bp, Artificial Sequence
  Description of Artificial Sequence primer
gacggatcgggagatctcc

SEQ ID NO: 14 - DNA, 22 bp, Artificial Sequence
  Description of Artificial Sequence primer
ccgcctcagaagccatagagcc

SEQ ID NO: 15 - DNA, 14455 bp, Artificial Sequence
  Description of Artificial Sequence plasmid
aagcttgggcagaaatggttgaactcccgagagtgtcctacacctaggggagaagcagccaaggggttgtttcccaccaaggacgacccgtctgcgcacaaacggatgagcccatcagacaaagacatattcattctctgctgcaaacttggcatagctctgctttgcctggggctattgggggaagttgcggttcgtgctcgcagggctctcacccttgactcttttaatagctcttctgtgcaagattacaatctaaacaattcggagaactcgaccttcctcctgaggcaaggaccacagccaacttcctcttacaagccgcatcgattttgtccttcagaaatagaaataagaatgcttgctaaaaattatatttttaccaataagaccaatccaataggtagattattagttactatgttaagaaatgaatcattatcttttagtactatttttactcaaattcagaagttagaaatgggaatagaaaatagaaagagacgctcaacctcaattgaagaacaggtgcaaggactattgaccacaggcctagaagtaaaaaagggaaaaaagagtgtttttgtcaaaataggagacaggtggtggcaaccagggacttataggggaccttacatctacagaccaacagatgcccccttaccatatacaggaagatatgacttaaattgggataggtgggttacagtcaatggctataaagtgttatatagatccctcccttttcgtgaaagactcgccagagctagacctccttggtgtatgttgtctcaagaagaaaaagacgacatgaaacaacaggtacatgattatatttatctaggaacaggaatgcacttttggggaaagattttccataccaaggaggggacagtggctggactaatagaacattattctgcaaaaactcatggcatgagttattatgaatagcctttattggcccaaccttgcggttcccagggcttaagtaagtttttggttacaaactgttcttaaaacgaggatgtgagacaagtggtttcctgacttggtttggtatcaaaggttctgatctgagctctgagtgttctattttcctatgttcttttggaatttatccaaatcttatgtaaatgcttatgtaaaccaagatataaaagagtgctgattttttgagtaaacttgcaacagtcctaacattcacctcttgtgtgtttgtgtctgttcgccatcccgtctccgctcgtcacttatccttcactttccagagggtccccccgcagaccccggcgaccctcaggtcggccgactgcggcagctggcgcccgaacagggaccctcggataagtgacccttgtctctatttctactatttggtgtttgtcttgtattgtctctttcttgtctggctatcatcacaagagcggaacggactcaccatagggaccaagctagcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctggtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagatgcatgctcgagcggccgcttctttattcttgggcaatgtatgaaaaagtgtaagaggatgtggcaaatatttcattaatgtagttgtggccagaccagtcccatgaaaatgacatagagtatgcacttggagttgtgtctcctgtttcctgtgtaccgtttagtgtaatggttagtgttacaggtttagttttgtctccgtttaagtaaacttgactgacaatgttacttttggcagttttaccgtgagattttggataagctgataggttaggcataaatccaacagcgtttgtataggctgtgccttcagtaagatctccatttctaaagttccaatattctgggtccaggaaggaattgtttagtagcactccattttcgtcaaatcttataataagatgagcactttgaactgttccagatattggagccaaactgcctttaacagccaaaactgaaactgtagcaagtatttgactgccacattttgttaagaccaaagtgagtttagcatctttctctgcatttagtctacagttaggagatggagctggtgtggtccacaaagttagcttatcattatttttgtttcctactgtaatggcacctgtgctgtcaaaactaaggccagttcctagtttaggaaccatagccttgtttgaatcaaattctaggccatggccaatttttgttttgaggggatttgtgtttggtgcattaggtgaaccaaattcaagcccatctcctgcattaatggctatggctgtagcgtcaaacatcaaccccttggcagtgcttaggttaacctcaagctttttggaattgtttgaagctgtaaacaagtaaaggcctttgttgtagttaatatccaagttgtgggctgagtttataaaaagagggccctgtcctagtcttagatttagttggttttgagcatcaaacggataactaacatcaagtataaggcgtctgttttgagaatcaatccttagtcctcctgctacattaagttgcatattgccttgtgaatcaaaacccaaggctccagtaactttagtttgcaaggaagtattattaatagtcacacctggaccagttgctacggtcaaagtgtttaggtcgtctgttacatgcaaaggagccccgtactttagtcctagttttccattttgtgtataaatgggctctttcaagtcaatgcccaagctaccagtggcagtagttagagggggtgaggcagtgatagtaagggtactgctatcggtggtggtgagggggcctgatgtttgcagggctagctttccttctgacactgtgaggggtccttgggtggcaatgctaagtttggagtcgtgcacggttagcggggcctgtgattgcatggtgagtgtgttgcccgcgaccattagaggtgcggcggcagccacagttagggcttctgaggtaactgtgaggggtgcagatatttccaggtttatgtttgacttggtttttttgagaggtgggctcacagtggttacattttgggaggtaaggttgccggcctcgtccagagagaggccgttgcccattttgagcgcaagcatgccattggaggtaactagaggttcggataggcgcaaagagagtaccccagggggactctcttgaaacccattgggggatacaaagggaggagtaagaaaaggcacagttggaggaccggtttccgtgtcatatggatacacggggttgaaggtatcttcagacggtcttgcgcgcttcatcttggatctcaagcctgccacacctcacctcgaccatccgccgtctcaagaccgcctactttaattacatcatcagcagcacctccgccagaaacaaccccgaccgccacccgctgccgcccgccacggtgctcagcctaccttgcgactgtgactggttagacgcctttctcgagaggttttccgatccggtcgatgcggactcgctcaggtccctcggtggcggagtaccgttcggaggccgacgggtttccgatccaagagtactggaaagaccgcgaagagtttgtcctcaaccgcgagcccaacagcgagctcgaattcagatccgagctcggtaccaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccataggggatcgggagatctcccgatccgtctatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagc

SEQ ID NO: 16 - DNA, 10610 bp, Artificial Sequence
  Description of Artificial Sequence plasmid
gacggatcgggagatccgcgcggtacacagaattcaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccggatcctctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatctgaattcgagctcgctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 17 - DNA, 24 bp, Artificial Sequence
  Description of Artificial Sequence Primer
tgtacaccggatccggcgcacacc

SEQ ID NO: 18 - DNA, 35 bp, Artificial Sequence
  Description of Artificial Sequence Primer
cacaacgagctcaattaattaattgccacatcctc

SEQ ID NO: 19 - protein, 4 aa, adenovirus
TLWT

SEQ ID NO: 20 - protein, 12 aa, adenovirus
PSASASASAPGS

SEQ ID NO: 21 - DNA, 37 bp, Artificial Sequence
  Description of Artificial Sequence primer
gcgcttaattaacatcatcaataatataccttatttt

SEQ ID NO: 22 - DNA, 327 bp, adenovirus
agatctgaattcgagctcgctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatct

SEQ ID NO: 23 - DNA, 32480 bp, adenovirus
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatctctagcatcgatgtcgacaagcttgaattcgattaatgtgagttagctcactcattaggcaccccaggctttacactttatgcttccggctcgtatgttgtgtggaattgtgagcggataacaatttcacacaggaaacagctatgaccatgattacgaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctggtaccggtgggtgaagaccagaaacagcacctcgaactgagccgcgatattgcccagcgtttcaacgcgctgtatggcgagatcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggccatgtctgcccgtatttcgcgtaaggaaatccattatgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcggaacttgtttattgcagcttataatggttacaaataaagcaatagcatcacaaatttcacaaataaagcatttttttcactgcattctagttgtggtttgtccaaactcatcaatgtatcttatcatgtctggatccagatctgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccttggattacatcaagatttaattaattgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccttgacaaaagaacccacactgattatgacacgcatactcggagctatgctaaccagcgtagccccgatgtaagctttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttcacagcggcagcctaacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtaacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatg

SEQ ID NO: 24 - DNA, 25 bp, Artificial Sequence
  Description of Artificial Sequence primer
ctcaacaattgtggatccgtactcc

SEQ ID NO: 25 - DNA, 25 bp, Artificial Sequence
  Description of Artificial Sequence primer
gtgctcagcagatcttgcgactgtg

SEQ ID NO: 26 - DNA, 25 bp, Artificial Sequence
  Description of Artificial Sequence primer
ggcgcgttcggatccactctcttcc

SEQ ID NO: 27 - DNA, 28 bp, Artificial Sequence
  Description of Artificial Sequence primer
ctacatgctaggcagatctcgttcggag

SEQ ID NO: 28 - DNA, 1240 bp, adenovirus
ggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatct

SEQ ID NO: 29 - DNA, 8383 bp, Artificial Sequence
  Description of Artificial Sequence plasmid pDV60
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaaagaagcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 30 - DNA, 7960 bp, Artificial Sequence
  Description of Artificial Sequence plasmid pDV67
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaaagaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 31 - DNA, 30 bp, Artificial Sequence; identical to SEQ ID NO: 5
  Description of Artificial Sequence primer

SEQ ID NO: 32 - DNA, 30 bp, Artificial Sequence
  Description of Artificial Sequence primer
cactatagcggccgcattctcagtcatctt

SEQ ID NO: 33 - DNA, 7989 bp, Artificial Sequence
  misc_feature 4242: N is any
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactgatttttaagaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccsnccntagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 34 - DNA, 7607 bp, Artificial Sequence
  Description of Artificial Sequence plasmid GRE5-E1-SV40-Hygro
tctagaagatccgctgtacaggatgttctagctactttattagatccgctgtacaggatgttctagctactttattagatccgctgtacaggatgttctagctactttattagatccgctgtacaggatgttctagctactttattagatccgtgtacaggatgttctagctactttattagatcgatctcctggccgttcggggtcaaaaaccaggtttggctataaaagggggtgggggcgcgttcgtcctcactctcttccgcatcgctgtctgcgagggccaggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattagatctaagcttctgcagctcgaggactcggtcgactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctcagctgactgcttaagtcgcaagccgaattggatccaattcggatcgatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagactcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattaattcgggg

SEQ ID NO: 35 - DNA, 11600 bp, Artificial Sequence
  Description of Artificial Sequence plasmid MMTV-E2a-SV40-Neo
gaattccgcattgcagagatattgtatttaagtgcctagctcgatacaataaacgccatttgaccattcaccacattggtgtgcacctccaagcttgggcagaaatggttgaactcccgagagtgtcctacacctaggggagaagcagccaaggggttgtttcccaccaaggacgacccgtctgcgcacaaacggatgagcccatcagacaaagacatattcattctctgctgcaaacttggcatagctctgctttgcctggggctattgggggaagttgcggttcgtgctcgcagggctctcacccttgactcttttaatagctcttctgtgcaagattacaatctaaacaattcggagaactcgaccttcctcctgaggcaaggaccacagccaacttcctcttacaagccgcatcgattttgtccttcagaaatagaaataagaatgcttgctaaaaattatatttttaccaataagaccaatccaataggtagattattagttactatgttaagaaatgaatcattatcttttagtactatttttactcaaattcagaagttagaaatgggaatagaaaatagaaagagacgctcaacctcaattgaagaacaggtgcaaggactattgaccacaggcctagaagtaaaaaagggaaaaaagagtgtttttgtcaaaataggagacaggtggtggcaaccagggacttataggggaccttacatctacagaccaacagatgcccccttaccatatacaggaagatatgacttaaattgggataggtgggttacagtcaatggctataaagtgttatatagatccctcccttttcgtgaaagactcgccagagctagacctccttggtgtatgttgtctcaagaagaaaaagacgacatgaaacaacaggtacatgattatatttatctaggaacaggaatgcacttttggggaaagattttccataccaaggaggggacagtggctggactaatagaacattattctgcaaaaactcatggcatgagttattatgaatagcctttattggcccaaccttgcggttcccagggcttaagtaagtttttggttacaaactgttcttaaaacgaggatgtgagacaagtggtttcctgacttggtttggtatcaaaggttctgatctgagctctgagtgttctattttcctatgttcttttggaatttatccaaatcttatgtaaatgcttatgtaaaccaagatataaaagagtgctgattttttgagtaaacttgcaacagtcctaacattcacctcttgtgtgtttgtgtctgttcgccatcccgtctccgctcgtcacttatccttcactttccagagggtccccccgcagaccccggcgaccctcaggtcggccgactgcggcagctggcgcccgaacagggaccctcggataagtgacccttgtctctatttctactatttggtgtttgtcttgtattgtctctttcttgtctggctatcatcacaagagcggaacggactcaccatagggaccaagctagcgcttctcgtcgcgtccaagaccctcaaagatttttggcacttcgttgagcgaggcgatatcaggtatgacagcgccctgccgcaaggccagctgcttgtccgctcggctgcggttggcacggcaggataggggtatcttgcagttttggaaaaagatgtgataggtggcaagcacctctggcacggcaaatacggggtagaagttgaggcgcgggttgggctcgcatgtgccgttttcttggcgtttggggggtacgcgcggtgagaataggtggcgttcgtaggcaaggctgacatccgctatggcgaggggcacatcgctgcgctcttgcaacgcgtcgcagataatggcgcactggcgctgcagatgcttcaacagcacgtcgtctcccacatctaggtagtcgccatgcctttcgtccccccgcccgacttgttcctcgtttgcctctgcgttgtcctggtcttgctttttatcctctgttggtactgagcggtcctcgtcgtcttcgcttacaaaacctgggtcctgctcgataatcacttcctcctcctcaagcgggggtgcctcgacggggaaggtggtaggcgcgttggcggcatcggtggaggcggtggtggcgaactcagagggggcggttaggctgtccttcttctcgactgactccatgatctttttctgcctataggagaaggaaatggccagtcgggaagaggagcagcgcgaaaccacccccgagcgcggacgcggtgcggcgcgacgtcccccaaccatggaggacgtgtcgtccccgtccccgtcgccgccgcctccccgggcgcccccaaaaaagcggatgaggcggcgtatcgagtccgaggacgaggaagactcatcacaagacgcgctggtgccgcgcacacccagcccgcggccatcgacctcggcggcggatttggccattgcgcccaagaagaaaaagaagcgcccttctcccaagcccgagcgcccgccatcaccagaggtaatcgtggacagcgaggaagaaagagaagatgtggcgctacaaatggtgggtttcagcaacccaccggtgctaatcaagcatggcaaaggaggtaagcgcacagtgcggcggctgaatgaagacgacccagtggcgcgtggtatgcggacgcaagaggaagaggaagagcccagcgaagcggaaagtgaaattacggtgatgaacccgctgagtgtgccgatcgtgtctgcgtgggagaagggcatggaggctgcgcgcgcgctgatggacaagtaccacgtggataacgatctaaaggcgaacttcaaactactgcctgaccaagtggaagctctggcggccgtatgcaagacctggctgaacgaggagcaccgcgggttgcagctgaccttcaccagcaacaagacctttgtgacgatgatggggcgattcctgcaggcgtacctgcagtcgtttgcagaggtgacctacaagcatcacgagcccacgggctgcgcgttgtggctgcaccgctgcgctgagatcgaaggcgagcttaagtgtctacacggaagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcacgacgcggcctgtccggccaatcagttttccggcaagtcttgcggcatgttcttctctgaaggcgcaaaggctcaggtggcttttaagcagatcaaggcttttatgcaggcgctgtatcctaacgcccagaccgggcacggtcaccttttgatgccactacggtgcgagtgcaactcaaagcctgggcacgcgccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacggcgcagacggcaagggtgggggtaaataatcacccgagagtgtacaaataaaagcatttgcctttattgaaagtgtctctagtacattatttttacatgtttttcaagtgacaaaaagaagtggcgctcctaatctgcgcactgtggctgcggaagtagggcgagtggcgctccaggaagctgtagagctgttcctggttgcgacgcagggtgggctgtacctggggactgttgagcatggagttgggtaccccggtaataaggttcatggtggggttgtgatccatgggagtttggggccagttggcaaaggcgtggagaaacatgcagcagaatagtccacaggcggccgagttgggcccctgtacgctttgggtggacttttccagcgttatacagcggtcgggggaagaagcaatggcgctacggcgcaggagtgactcgtactcaaactggtaaacctgcttgagtcgctggtcagaaaagccaaagggctcaaagaggtagcatgtttttgagtgcgggttccaggcaaaggccatccagtgtacgcccccagtctcgcgaccggccgtattgactatggcgcaggcgagcttgtgtggagaaacaaagcctggaaagcgcttgtcataggtgcccaaaaaatatggcccacaaccaagatctttgacaatggctttcagttcctgctcactggagcccatggcggcagctgttgttgatgttgcttgcttctttatgttgtggcgttgccggccgagaagggcgtgcgcaggtacacggtttcgatgacgccgcggtgcggccggtgcacacggaccacgtcaaagacttcaaacaaaacataaagaagggtgggctcgtccatgggatccatatatagggcccgggttataattacctcaggtcgacctcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccaggaagctcctctgtgtcctcataaaccctaacctcctctacttgagaggacattccaatcataggctgcccatccaccctctgtgtcctcctgttaattaggtcacttaacaaaaaggaaattgggtaggggtttttcacagaccgctttctaagggtaattttaaaatatctgggaagtcccttccactgctgtgttccagaagtgttggtaaacagcccacaaatgtcaacagcagaaacatacaagctgtcagctttgcacaagggcccaacaccctgctcatcaagaagcactgtggttgctgtgttagtaatgtgcaaaacaggaggcacattttccccacctgtgtaggttccaaaatatctagtgttttcatttttacttggatcaggaacccagcactccactggataagcattatccttatccaaaacagccttgtggtcagtgttcatctgctgactgtcaactgtagcattttttggggttacagtttgagcaggatatttggtcctgtagtttgctaacacaccctgcagctccaaaggttccccaccaacagcaaaaaaatgaaaatttgacccttgaatgggttttccagcaccattttcatgagttttttgtgtccctgaatgcaagtttaacatagcagttaccccaataacctcagttttaacagtaacagcttcccacatcaaaatatttccacaggttaagtcctcatttaaattaggcaaaggaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccact

SEQ ID NO: 36 - DNA, 53 bp, Artificial Sequence
  Description of Artificial Sequence primer
gtcactcgaggactcggtcgactgaaaatgagacatattatctgccacggacc

SEQ ID NO: 37 - DNA, 36 bp, Artificial Sequence
  Description of Artificial Sequence primer
cgagatcgatcacctccggtacaaggtttggcatag

SEQ ID NO: 38 - DNA, 37 bp, Artificial Sequence
  Description of Artificial Sequence primer
catgaagatctggaaggtgctgaggtacgatgagacc

SEQ ID NO: 39 - DNA, 51 bp, Artificial Sequence
  Description of Artificial Sequence primer
gcgacttaagcagtcagctgagacagcaagacacttgcttgatccaaatcc

SEQ ID NO: 40 - DNA, 38 bp, Artificial Sequence
  Description of Artificial Sequence primer
cacgaattcgtcagcgcttctcgtcgcgtccaagaccc

SEQ ID NO: 41 - DNA, 32 bp, Artificial Sequence
  Description of Artificial Sequence primer
caccccggggaggcggcggcgacggggacggg

SEQ ID NO: 42 - DNA, 7231 bp, Artificial Sequence
  Description of Artificial Sequence plasmid pDV80
ctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacgagatccgtactccgccgccgagggacctgagcgagtccgcatcgaccggatcggaaaacctctcgagaaaggcgtctaaccagtcacagtcgcaagatccaagatgaagcgcgcccgccccagcgaagatgacttcaaccccgtctacccctatggctacgcgcggaatcagaatatccccttcctcactcccccctttgtctcctccgatggattcaaaaacttcccccctggggtactgtcactcaaactggctgatccaatcaccattaccaatggggatgtatccctcaaggtgggaggtggtctcactttgcaagatggaagcctaactgtaaaccctaaggctccactgcaagttaatactgataaaaaacttgagcttgcatatgataatccatttgaaagtagtgctaataaacttagtttaaaagtaggacatggattaaaagtattagatgaaaaaagtgctgcggggttaaaagatttaattggcaaacttgtggttttaacaggaaaaggaataggcactgaaaatttagaaaatacagatggtagcagcagaggaattggtataaatgtaagagcaagagaagggttgacatttgacaatgatggatacttggtagcatggaacccaaagtatgacacgcgcacactttggacaacaccagacacatctccaaactgcacaattgctcaagataaggactctaaactcactttggtacttacaaagtgtggaagtcaaatattagctaatgtgtctttgattgtggtcgcaggaaagtaccacatcataaataataagacaaatccaaaaataaaaagttttactattaaactgctatttaataagaacggagtgcttttagacaactcaaatcttggaaaagcttattggaactttagaagtggaaattccaatgtttcgacagcttatgaaaaagcaattggttttatgcctaatttggtagcgtatccaaaacccagtaattctaaaaaatatgcaagagacatagtttatggaactatatatcttggtggaaaacctgatcagccagcagtcattaaaactacctttaaccaagaaactggatgtgaatactctatcacatttaactttagttggtccaaaacctatgaaaatgttgaatttgaaaccacctcttttaccttctcctatattgcccaagaatgaaagagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 43 - DNA, 48 bp, Artificial Sequence
  Description of Artificial Sequence primer
tgtcttggatccaagatgaagcgcgcccgccccagcgaagatgacttc

SEQ ID NO: 44 - DNA, 28 bp, Artificial Sequence
  Description of Artificial Sequence primer
aaacacggcggccgctctttcattcttg

SEQ ID NO: 45 - protein, 17 aa, Ad 37 N-terminus
MSKRLRVEDDFNPVYPY

SEQ ID NO: 46 - protein, 6 aa, artificial sequence
  Description of Artificial Sequence primer
KRARPS

SEQ ID NO: 47 - protein, 17 aa, Ad5 modified N-terminus
MKRARPSEDTFNPVYPY

SEQ ID NO: 48 - DNA, 24 bp, Artificial Sequence
  Description of Artificial Sequence primer
ggatccatgggatacttggtagca

SEQ ID NO: 49 - DNA, 30 bp, Artificial Sequence
  Description of Artificial Sequence primer
gcaactcgagtcattcttgggcaatatagg

SEQ ID NO: 50 - DNA, 26 bp, Artificial Sequence
  Description of Artificial Sequence primer
cgcgctgactcttaaggactagtttc